(19) United States
(12) Patent Application Publication
(10) Pub. No.: US 2020/0318190 A1
(43) Pub. Date: Oct. 8, 2020

(54) STRATIFICATION OF RISK OF VIRUS ASSOCIATED CANCERS

(71) Applicant: GRAIL, Inc., Menlo Park, CA (US)

(72) Inventors: Yuk-Ming Dennis LO, Hong Kong SAR (CN); Rossa Wai Kwun CHIU, Hong Kong SAR (CN); Kwan Chee CHAN, Hong Kong SAR (CN); Peiyong JIANG, Hong Kong SAR (CN); Wai Kei LAM, Hong Kong SAR (CN); Lu JI, Hong Kong SAR (CN)

(21) Appl. No.: 16/837,476

(22) Filed: Apr. 1, 2020

Related U.S. Application Data
(60) Provisional application No. 62/828,224, filed on Apr. 2, 2019.
(60) Provisional application No. 62/961,517, filed on Jan. 15, 2020.

(51) Int. Cl. C12Q 1/6883

(52) U.S. Cl. CPC C12Q 1/6883; G01N 2800/52; C12Q 2600/156; C12Q 2600/154

(57) ABSTRACT

Provided herein are methods and systems for stratifying risk for a subject to develop a pathogen-associated disorder based on analysis of cell-free nucleic acid molecules from a biological sample of the subject. In various examples, screening frequency is determined based on the risk analysis. Also provided herein are methods and systems for analyzing variant patterns of a pathogen genome in cell-free nucleic acid molecules.

### CROSS-REFERENCE

This application claims the benefits of U.S. Provisional Application No. 62/961,517, filed Jan. 15, 2020, and U.S. Provisional Application No. 62/828,224, filed Apr. 2, 2019, each of which is incorporated herein by reference in its entirety.

### BACKGROUND

Many diseases and conditions can be associated with infection of pathogens such as viruses. Nasopharyngeal cancer (NPC) is one of the most prevalent cancers in the southern parts of China and Southeast Asia and the pathogenesis of NPC can be closely associated with Epstein-Barr virus (EBV) infection. In high incidence regions for NPC, almost all NPC tumors would harbor the EBV genome. Based on the close relationship between EBV and NPC, plasma EBV DNA has been developed as a biomarker of NPC. Using real-time polymerase chain reaction (PCR) analysis, the detection of plasma EBV DNA was shown to have a sensitivity of 95% and specificity of 93% for detecting NPC (Lo et al. Cancer Res. 1999; 59:1188-91). There can be significant clinical benefits to develop non-invasive or minimally invasive diagnostic assays for stratifying risks for these pathogen-associated disorders based on analysis of cell-free nucleic acid molecules from the pathogen in biological samples.

### SUMMARY

In some aspects, provided herein is a method of screening a pathogen-associated disorder in a subject, comprising: receiving data from a first assay performed at a first time point that comprises determining a characteristic of cell-free nucleic acid molecules from a pathogen in a biological sample of the subject, wherein the characteristic of the cell-free nucleic acid molecules from the pathogen comprises amount, methylation status, variant pattern, fragment size, or relative abundance as compared to cell-free nucleic acid molecules from the subject in the biological sample, and wherein the characteristic indicates a risk for the subject to develop the pathogen-associated disorder; and determining, based on the characteristic, a second time point at which a second assay is performed to screen for the pathogen-associated disorder in the subject, wherein an interval between the first time point and the second time point inversely correlates with the risk.

In some aspects, provided herein is a method of prognosticating a pathogen-associated disorder in a subject, comprising: receiving data from a first assay that comprises determining a characteristic of cell-free nucleic acid molecules from a pathogen in a biological sample of the subject, wherein the characteristic of the cell-free nucleic acid molecules from the pathogen comprises amount, methylation status, variant pattern, fragment size, or relative abundance as compared to cell-free nucleic acid molecules from the subject in the biological sample; and generating a report indicative of a risk for the subject to develop the pathogen-associated disorder based on the characteristic of the cell-free nucleic acid molecules from the pathogen, and one or more factors of age of the subject, smoking habit of the subject, family history of the pathogen-associated disorder of the subject, genotypic factors of the subject, ethnicity of the subject, or dietary history of the subject.

In some cases, result of the first assay does not result in a medical treatment of the subject for the pathogen-associated disorder. In some cases, the medical treatment comprises treatment with therapeutic agents, radiotherapy, or surgical treatment. In some cases, the subject is diagnosed as not having the pathogen-associated disorder before the determining a second time point by a clinical diagnostic examination that has a false positive rate below 1%. In some cases, the clinical diagnostic examination comprises physical examination, invasive biopsy, endoscopy, magnetic resonance imaging, positive emission tomography, computed tomography, or x-ray imaging. In some cases, the clinical diagnostic examination comprises invasive biopsy that comprises histological analysis, cytological analysis, or cellular nucleic acid analysis. In some cases, the interval is at least about 2 months, 4 months, 6 months, 8 months, 10 months, or 12 months. In some cases, the interval is at least about 12 months.

In some cases, the method further comprises performing the first assay. In some cases, the performing the first assay comprises: (i) obtaining a first biological sample from the subject; and (ii) measuring a first amount of cell-free nucleic acid molecules from the pathogen in the first biological sample. In some cases, the measuring the first amount comprises measuring a copy number of the cell-free nucleic acid molecules from the pathogen in the first biological sample. In some cases, the measuring comprises polymerase chain reaction (PCR). In some cases, the measuring comprises quantitative PCR (qPCR). In some cases, the first amount comprises measuring a first percentage of the cell-free nucleic acid molecules from the pathogen in the first biological sample. In some cases, the first assay further comprises: (iii) if the first amount is above a threshold, obtaining a second biological sample from the subject, and measuring a second amount of cell-free nucleic acid molecules from the pathogen in the second biological sample. In some cases, the second biological sample is obtained about 4 weeks after the first biological sample. In some cases, the interval between the first time point and the second time point is shorter if both the first amount and the second copy number are above the threshold as compared to an interval if the second amount is below the threshold. In some cases, the interval between the first time point and the second time point is longer if the first amount is below the threshold as compared to an interval if the first amount is above the threshold. In some cases, the interval between the first time point and the second time point is about 1 year if both the first amount and the second amount are above the threshold. In some cases, the interval between the first time point and the second time point is about 2 years if the second amount is below the threshold. In some cases, the interval between the first time point and the second time point is about 4 years if the first amount is below the threshold. In some cases, the first assay comprises: determining a methylation status of the cell-free nucleic acid molecules from the pathogen in the biological sample. In some cases, the determining the methylation status comprises treatment of the cell-free nucleic acid molecules in the biological sample with a methylation-sensitive restriction enzyme or bisulfite. In some cases, the determining the methylation status comprises performing a methylation-aware sequencing of cell-free nucleic acids in the biological sample of the subject. In some cases, the methylation-aware sequencing comprises bisulfite conversion of unmethylated cytosine to uracil. In some cases, the methylation-aware sequencing comprises treatment with a methylation-sensitive restriction enzyme. In some cases, the first assay comprises: determining a fragment size distribution of the cell-free nucleic acid molecules from the pathogen in the biological sample. In some cases, the determining the fragment size distribution comprises performing sequencing on cell-free nucleic acid molecules in the biological sample, and determining a fragment size of the cell-free nucleic acid molecules from the pathogen in the biological sample based on sequence reads mapped to the reference genome of the pathogen.

In some cases, the first assay comprises: determining a variant pattern of the cell-free nucleic acid molecules from the pathogen in the biological sample. In some cases, the determining the variant pattern comprises performing sequencing on cell-free nucleic acid molecules in the biological sample, and determining the variant pattern of the cell-free nucleic acid molecules from the pathogen in the biological sample based on sequence reads mapped to the reference genome of the pathogen. In some cases, the variant pattern of the cell-free nucleic acid molecules from the pathogen comprises single nucleotide variations. In some cases, the identifying the variant pattern comprises: determining a similarity level between the sequence reads mapped to the reference genome of the pathogen and a disorder-related reference genome of the pathogen. In some cases, the disorder-related reference genome of the pathogen comprises a genome of the pathogen identified in a diseased tissue. In some cases, the determining the similarity level comprises: segregating the reference genome of the pathogen into a plurality of bins; and determining a similarity index for each of the plurality of bins against the disorder-related reference genome of the pathogen, wherein the similarity index correlates with a proportion of the variant sites, within the respective bin, at which at least one of the sequence reads mapped to the reference genome of the pathogen has a same nucleotide variant as the disorder-related reference genome of the pathogen. In some cases, the disorder-related reference genome of the pathogen comprises a plurality of disorder-related reference genomes of the pathogen, and wherein the determining the similarity level comprises: determining a respective similarity index for each of the plurality of bins against each of the plurality of disorder-related reference genomes of the pathogen; and determining a bin score for each of the plurality of bins based on a proportion of the plurality of disorder-related reference genomes, against which the respective similarity index within the respective bin is above a cutoff value. In some cases, each of the plurality of bins has a length of about 100, 200, 300, 400, 500, 600, 700, 800, 900, or 1000 bp. In some cases, the first assay comprises determining the methylation status, the fragment size distribution, or the variant pattern of the cell-free nucleic acid molecules from the pathogen in the biological sample.

In some cases, the method further comprises calculating a risk score for the subject to develop the pathogen-associated disorder using a classifier applied to a data input comprising the characteristic of the cell-free nucleic acid molecules from the pathogen in the biological sample, wherein the classifier is configured to apply a function to the data input comprising the characteristic of the cell-free nucleic acid molecules from the pathogen in the biological sample to generate an output comprising the risk score that evaluates the risk for the subject to develop the disorder. In some cases, the classifier is trained with a labeled dataset.

In some cases, the method further comprises performing the second assay at the second time point. In some cases, the second assay is same as the first assay. In some cases, the second assay comprises an assay of cell-free nucleic acid molecules from the subject, an invasive biopsy of the subject, endoscopic examination of the subject, or magnetic resonance imaging examination of the subject.

In some aspects, provided herein is a method of analyzing nucleic acid molecules from a biological sample of a subject, comprising: obtaining, in a computer system, sequence reads of cell-free nucleic acid molecules from the biological sample of the subject, wherein the biological sample comprises cell-free nucleic acid molecules from the subject and potentially from a pathogen; aligning, in the computer system, the sequence reads of the cell-free nucleic acid molecules to a reference genome of the pathogen; and identifying, in the computer system, a variant pattern of the cell-free nucleic acid molecules from the pathogen, wherein the variant pattern characterizes a nucleotide variant of the sequence reads mapped to the reference genome of the pathogen at each of a plurality of variant sites on the reference genome of the pathogen, wherein the plurality of variant sites comprises at least 30 sites across the reference genome of the pathogen, and wherein the variant pattern indicates a status of, or a risk for, a pathogen-associated disorder in the subject.

In some cases, the plurality of variant sites comprises at least 40, at least 50, at least 60, at least 70, at least 80, at least 90, at least 100, at least 200, at least 300, at least 400, at least 500, at least 600, at least 700, at least 800, at least 900, at least 1000, at least 1100, or at least 1200 sites across the reference genome of the pathogen. In some cases, the plurality of variant sites comprises the plurality of variant sites comprises at least 600 sites across the reference genome of the pathogen. In some cases, the plurality of variant sites comprises the plurality of variant sites comprises about 660 sites across the reference genome of the pathogen. In some cases, the plurality of variant sites comprises the plurality of variant sites comprises at least 1000 sites across the reference genome of the pathogen. In some cases, the plurality of variant sites comprises about 1100 sites across the reference genome of the pathogen. In some cases, the plurality of variant sites consists of all sites at which the sequence reads mapped to the reference genome of the pathogen have a different nucleotide variant than the reference genome of the pathogen. In some cases, the aligning the sequence reads is configured to allow a maximum mismatch of 10, 9, 8, 7, 6, 5, 4, 3, 2, or 1 bases between the sequence reads mapped to the reference genome of the pathogen and the reference genome of the pathogen. In some cases, the aligning the sequence reads is configured to allow a maximum mismatch of 2 bases between the sequence reads mapped to the reference genome of the pathogen and the reference genome of the pathogen. In some cases, the method further comprises: (d) diagnosing, prognosticating, or monitoring the pathogen-associated disorder in the subject based on the variant pattern of the sequence reads mapped to the reference genome of the pathogen. In some cases, the variant pattern of the cell-free nucleic acid molecules from the pathogen comprises single nucleotide variations. In some cases, the identifying the variant pattern comprises: determining a similarity level between the sequence reads mapped to the reference genome of the pathogen and a disorder-related reference genome of the pathogen. In some cases, the disorder-related reference genome of the pathogen comprises a genome of the pathogen identified in a diseased tissue. In some cases, the determining the similarity level comprises: segregating the reference genome of the pathogen into a plurality of bins; and determining a similarity index for each of the plurality of bins against the disorder-related reference genome of the pathogen, wherein the similarity index correlates with a proportion of the variant sites, within the respective bin, at which at least one of the sequence reads mapped to the reference genome of the pathogen has a same nucleotide variant as the disorder-related reference genome of the pathogen. In some cases, the disorder-related reference genome of the pathogen comprises a plurality of disorder-related reference genomes of the pathogen, and wherein the determining the similarity level comprises: determining a respective similarity index for each of the plurality of bins against each of the plurality of disorder-related reference genomes of the pathogen; and determining a bin score for each of the plurality of bins based on a proportion of the plurality of disorder-related reference genomes, against which the respective similarity index within the respective bin is above a cutoff value. In some cases, the cutoff value is about 0.9. In some cases, each of the plurality of bins has a length of about 100, 200, 300, 400, 500, 600, 700, 800, 900, or 1000 bp. In some cases, the method further comprises: calculating a risk score for the subject to develop the pathogen-associated disorder using a classifier applied to a data input comprising the variant pattern of the cell-free nucleic acid molecules from the pathogen, wherein the classifier is configured to apply a function to the data input comprising the variant pattern of the cell-free nucleic acid molecules from the pathogen to generate an output comprising the risk score that evaluates the risk for the subject to develop the disorder. In some cases, the classifier is trained with a labeled dataset. In some cases, the classifier comprises a mathematical model using Naïve Bayes model, logistics regression, random forest, decision tree, gradient boosting tree, neural network, deep learning, linear/kernel support vector machine (SVM), linear/non-linear regression, or linear discriminative analysis.

In some cases, the pathogen is a virus. In some cases, the virus is Epstein-Barr virus (EBV). In some cases, the pathogen-associated disorder comprises nasopharyngeal cancer, NK cell lymphoma, Burkitt's lymphoma, post-transplant lymphoproliferative disorders, or Hodgkin's lymphoma. In some cases, the variant pattern of the cell-free nucleic acid molecules from the pathogen characterizes nucleotide variant of the sequence reads mapped to the referenced genome of the pathogen at each of a plurality of variant sites that comprises at least 30, 40, 50, 100, 150, 200, 250, 300, 350, 400, 450, 500, 550, or 600 sites selected from genomic sites as set forth in Table 6 relative to EBV reference genome (AJ507799.2). In some cases, the plurality of variant sites comprises a genomic site as set forth in Table 6 relative to EBV reference genome (AJ507799.2). In some cases, the variant pattern of the cell-free nucleic acid molecules from the pathogen characterizes nucleotide variant of the sequence reads mapped to the referenced genome of the pathogen at each of the plurality of variant sites that are randomly selected from genomic sites as set forth in Table 6 relative to EBV reference genome (AJ507799.2). In some cases, the variant pattern of the cell-free nucleic acid molecules from the pathogen characterizes nucleotide variant of the sequence reads mapped to the referenced genome of the pathogen at each of the plurality of variant sites that comprise at least 30, 40, 50, 100, 150, 200, 250, 300, 350, 400, 450, 500, 550, or 600 sites randomly selected from genomic sites as set forth in Table 6 relative to EBV reference genome (AJ507799.2).

In some cases, the virus is human papillomavirus (HPV). In some cases, the pathogen-associated disorder comprises cervical cancer, oropharyngeal cancer, or head and neck cancers. In some cases, the virus is hepatitis B virus (HBV). In some cases, the pathogen-associated disorder comprises cirrhosis or hepatocellular carcinoma (HCC). In some cases, the variant pattern indicates a status of a pathogen-associated disorder in the subject, the status of the pathogen-associated disorder comprises a presence of the pathogen-associated disorder in the subject, an amount of tumor tissue in the subject, a size of the tumor tissue in the subject, a stage of tumor in the subject, a tumor load in the subject, or a presence of tumor metastasis in the subject. In some cases, the biological sample is selected from the group consisting of: whole blood, blood plasma, blood serum, urine, cerebrospinal fluid, buffy coat, vaginal fluid, vaginal flushing fluid, saliva, oral rinse fluid, nasal flushing fluid, a nasal brush sample and a combination thereof.

In some aspects, provided herein is a non-transitory computer-readable medium comprising machine executable code that, upon execution by one or more computer processors, implements any of the methods above.

In some aspects, provided herein is a computer product comprising a non-transitory computer readable medium storing a plurality of instructions for controlling a computer system to perform operations of any of the methods above.

In some aspects, provided herein is a system comprising: the computer product as described herein; and one or more processors for executing instructions stored on the computer readable medium.

In some aspects, provided herein is a system comprising means for performing any of the methods above.

In some aspects, provided herein is a system configured to perform any of the above methods.

In some aspects, provided herein is a system comprising modules that respectively perform the steps of any of the above methods.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features described herein are set forth with particularity in the appended claims. A better understanding of the features and advantages described herein will be obtained by reference to the following detailed description that sets forth illustrative embodiments, in which the principles described herein are utilized, and the accompanying drawings of which:

FIG. 1 is a diagram of the design of a NPC screening study over a cohort of over 20,000 subjects.

FIG. 2 shows an exemplary schematic of a NPC screening regimen according to the present disclosure.

FIG. 3 summarizes phylogenetic tree analysis based on the EBV variant profiles of samples from NPC patients and non-NPC subjects.

FIG. 4 summarizes phylogenetic tree analysis based on the EBV variant profiles of samples from NPC patients and non-NPC subjects excluding 29 reported variants.

FIG. 5 summarizes phylogenetic tree analysis based on the EBV variant profiles of samples from NPC patients, non-NPC subjects, and pre-NPC subjects.

FIG. 6 summarizes phylogenetic tree analysis based on the EBV variant profiles of samples from NPC patients, non-NPC subjects, and pre-NPC subjects excluding 29 reported variants.

FIG. 7 illustrates the principle of block-based variant pattern analysis.

FIG. 8 summarizes block-based analysis of EBV DNA variant patterns of 13 NPC, 16 non-NPC and 4 pre-NPC samples.

FIG. 9 summarizes block-based analysis of EBV DNA variant patterns of 13 NPC, 16 non-NPC and 4 pre-NPC samples excluding 29 reported variants.

FIG. 10A shows the NPC risk score calculated using a trained classifier based on the analysis of all EBV variants using block-based variant analysis. FIG. 10B shows the NPC risk score calculated using the trained classifier based on the analysis of 29 reported EBV variants.

FIG. 10C shows the NPC risk score calculated using the trained classifier based on the analysis of all EBV variants using block-based variant analysis but excluding 29 reported variants.

FIG. 11 summarizes methylation levels of NPC patients and non-NPC subjects with transiently positive EBV DNA or persistently positive EBV DNA.

FIG. 12 is a schematic illustrating the size changes of plasma DNA of a non-cancer subject with positive plasma EBV DNA induced by methylation-sensitive enzyme digestion. The filled and unfilled lollipops represent methylated and unmethylated CpG sites, respectively. Yellow horizontal bars represent the plasma EBV DNA molecules. With the enzyme digestion, the size distribution shifts to the left side.

FIG. 13 is a schematic illustrating the size changes of plasma DNA of a NPC patient with positive EBV DNA induced by methylation-sensitive enzyme digestion. The filled and unfilled lollipops represent methylated and unmethylated CpG sites, respectively. Yellow horizontal bars represent the plasma EBV DNA molecules. With the enzyme digestion, the size distribution shifts to the left side.

FIG. 14 shows the size profiles of plasma EBV DNA with and without in-silico digestion with methylation-sensitive restriction enzyme HpaII.

FIG. 15 shows the cumulative size profiles of plasma EBV DNA with and without methylation-sensitive restriction enzyme digestion for a NPC patient and a subject without NPC.

FIG. 16A is a schematic demonstrating three hypothetical sites A, B and C in the training set of 661 SNV sites across the EBV genome which were associated with NPC. The NPC risk score of a test sample was formulated to be determined by the genotypic patterns over the subset of these 661 SNV sites which were covered by plasma EBV DNA reads (e.g., with available genotypic information). From the plasma sequencing data of the test sample, the genotypic information was only available for the sites A and C but not for the site B as the site B was not covered by any sequenced EBV DNA reads. FIG. 16B is a schematic demonstrating the weighting of genotypes at the sites A and C by analyzing the genotypes over these 2 sites for all the 63 NPC samples and 88 non-NPC samples in the training set. A logistic regression model was constructed to inform the weighting of the high-risk genotypes at the sites A and C. FIG. 16C is a schematic demonstrating the process where the NPC risk score of the test sample was derived based on its genotypes at the sites A and C, weighted by their corresponding coefficients deduced from the training model. FIG. 16D shows distribution of 5678 SNVs across the EBV genome from NPC and non-NPC samples in the training set (the total number of variants in a sliding window of 1000 nucleotides across the EBV genome is shown).

FIGS. 17A and 17B are graphs summarizing NPC risk scores in the training set using the leave one-out approach. FIG. 17A shows NPC risk scores of NPC and non-NPC plasma samples in the training set. FIG. 17B shows ROC curve analysis for the differentiation of NPC and non-NPC samples by the NPC risk score analysis.

FIGS. 18A and 18B are graphs summarizing NPC risk scores in the testing set. FIG. 18A shows NPC risk scores of NPC and non-NPC plasma samples in the testing set. FIG. 18B shows ROC curve analysis for the differentiation of NPC and non-NPC samples by the NPC risk score analysis.

FIGS. 19A and 19B are graphs summarizing NPC risk analysis by analyzing the genotypic patterns over EBER region. FIG. 19A shows NPC risk scores of NPC and non-NPC plasma samples in the testing set by analyzing the genotypic patterns over EBER region. FIG. 19B shows ROC curve analysis for the differentiation of NPC and non-NPC samples based on the NPC risk score analysis over EBER region.

FIGS. 20A and 20B are graphs summarizing NPC risk by analyzing the genotypic patterns over BALF2 region. FIG. 20A shows NPC risk scores of NPC and non-NPC plasma samples in the testing set by analyzing the genotypic patterns over BALF2 region. FIG. 20B shows ROC curve analysis for the differentiation of NPC and non-NPC samples based on the NPC risk score analysis over BALF2 region.

FIG. 21 shows a computer control system that can be programmed or otherwise configured to implement methods provided herein.

FIG. 22 shows a diagram of the methods and systems as disclosed herein.

### DETAILED DESCRIPTION

### Overview

In aspects, provided herein are methods and systems for screening for a pathogen-associated disorder in a subject. The methods and systems can provide evaluation of the risk for the subject to develop the pathogen-associated disorder based on a characteristic of cell-free nucleic acid molecules from the pathogen in a biological sample from the subject. Among others, the risk prediction can enable determination of appropriate screening frequency. Appropriate and timely follow-up screening can not only save the cost for the subject, but also enable early discovery of disorders. For instance, shift in stage distribution to earlier stages in EBV-NPC can result in a significant improvement in progression-free survival of the NPC patients.

The risk for the subject to develop the pathogen-associated disorder can refer to the possibility the subject is disposed to develop the pathogen-associated disorder. In some cases, the risk as described herein refers to the possibility that the pathogen-associated disorder develops in the subject into a state that can be clinically detected (“clinically detectable disorder”) at a future time point. In some cases, the subject is screened at a first time point by a screening assay that tests the cell-free nucleic acid molecules from a pathogen in a biological sample from the subject, and while the subject is diagnosed as not having a clinically detectable pathogen-associated disorder at the first time point, the characteristic of the cell-free nucleic acid molecules from the pathogen in the biological sample from the subject can indicate a risk for the subject to have the clinically detectable disorder at a future time point.

Clinically detectable disorder can refer to a disorder manifesting pathological symptoms that can be detected via one or more well-established clinical diagnostic examinations. In some cases, the well-established clinical diagnostic examinations include medical tests/assays that have a low false positive detection rate of the pathogen-associated disorder, such as, below 30%, 20%, 10%, 8%, 7%, 6%, 5%, 4%, 3%, 2.5%, 2%, 1%, 0.8%, 0.5%, 0.25%, 0.15%, 0.1%, 0.08%, 0.05%, 0.02%, 0.01%, 0.005%, 0.002%, 0.001%, or even lower. The well-established clinical diagnostic examinations include medical tests/assays can also have a high sensitivity of detecting the pathogen-associated disorder, such as, at least 30%, 40%, 50%, 60%, 70%, 80%, 85%, 90%, 92%, 94%, 95%, 96%, 97%, 98%, 99%, or 99.5%, or 100%. In some cases, the pathogen-associated disorder is a pathogen-associated proliferative disorder, such as, cancer, and the cancer can be clinically diagnosed with high confidence and low false positive ratio by one or more of invasive biopsy followed by histological or other exam of the biopsy tissue (e.g., tissue analysis, cellular examination, such as cellular DNA or protein analysis), imaging examination, e.g., X-ray, magnetic resonance imaging (MRI), positron emission tomography (PET), or computed tomography (CT), or PET-CT, laboratory tests (e.g., blood or urine tests), or physical exams. The diagnosis of the pathogen-associated disorder can be given by a certified medical doctor based on the results of the aforementioned or other well-established clinical examinations. In some cases, the result of the first screening assay does not result in a medical treatment of the subject for the pathogen-associated disorder, as the subject is diagnosed as not having the disorder by a well-established clinical diagnostic examination.

Based on the evaluated risk, in some cases, the methods include determining a frequency of screening assays for the pathogen-associated in the subject. The frequency of the screening assays can be correlated with the risk, and the interval between two screening assays, e.g., a screening assay as described herein and a subsequent follow-up screening assay, can be inversely correlated with the risk. In some cases, the methods include receiving data from a first screening assay that is performed at a first time point. The first screening assay can include determining a characteristic of cell-free nucleic acid molecules from the pathogen in a biological sample from the subject. For instance, the first screening assay includes obtaining a biological sample from the subject, and the biological sample includes cell-free nucleic acid molecules, e.g., cell-free DNA, from the subject and potentially from the pathogen. The first screening assay can also include determining a characteristic of the cell-free nucleic acid molecule from the pathogen in the biological sample. Non-limiting characteristic of the cell-free nucleic acid molecules from the pathogen that can be used in the methods and systems provided herein include amount (e.g., copy number or percentage), methylation status, fragment size, variant pattern, and relative abundance as compared to cell-free nucleic acid molecules from the subject in the biological sample. As described herein, the time point with respect to an examination or assay performed on a subject or a biological sample from the subject can refer to the time point the subject is subject to the examination or the time point the biological sample is obtained from the subject rather than the time point the actual assay is performed on the biological sample.

In some cases, methods provided herein comprise (a) receiving data from a first assay performed at a first time point that comprises determining a characteristic of cell-free nucleic acid molecules from a pathogen in a biological sample of the subject, wherein the characteristic of the cell-free nucleic acid molecules from the pathogen comprises amount (e.g., copy number or percentage), methylation status, variant pattern, fragment size, or relative abundance a s compared to cell-free nucleic acid molecules from the subject in the biological sample, and wherein the characteristic indicates a risk for the subject to develop the pathogen-associated disorder; and (b) determining, based on the characteristic, a second time point at which a second assay is performed to screen for the pathogen-associated disorder in the subject, wherein an interval between the first time point and the second time point inversely correlates with the risk.

The one or more characteristic of the cell-free nucleic acid molecules in the biological sample of the subject as described herein, in some cases, enables a non-invasive approach to evaluating the status of the pathogen-associated disorder (e.g., cancer) in the subject or the risk for the subject to develop the pathogen-associated disorder in the future. Without wishing to be bound by a certain theory, there can be at least two possible scenarios that underlie the association between the one or more characteristics of the cell-free nucleic acid molecules that can be used in the methods and systems and the risk for the subject to develop the pathogen-associated disorder. In one possible scenario, the diseased tissue suffering the pathogen-associated disorder, e.g., the pathogen-associated tumor, can already be present at the time of the initial screening (e.g., the first screening assay). However, the size of the diseased tissue, e.g., the tumor, can be too small to be picked up by other classical medical examination approaches, e.g., approaches having false positive rate of detecting the pathogen-associated disorder below 10%, 5%, 2%, 1%, 0.5%, 0.1%, or 0.05%, such as endoscopy and magnetic resonance imaging (MRI). With the development of the disorder, for instance, the growth of the diseased tissue, e.g., the tumor, in size, the more advanced diseased tissue, for instance, the enlarged tissue (e.g., the enlarged tumor), can then be detected in a subsequent screening (second screening assay). Another possible scenario can be: the nucleic acid molecules of the pathogen, e.g., EBV DNA, can be released by cells that are in preliminary diseased state, for instance, pre-malignant cells, and those cells can later on potentially develop into diseased cells, e.g., cancer cells. Irrespective of the exact scenario underlying the association, the subject matter described here can be used to stratify subjects for their risk of having clinically detectable NPC subsequently.

In some cases, The actual time intervals used for specific screening programs as described herein are adjusted according to health economic considerations (e.g., the cost of the screening), subject preference (e.g., a more frequent screening interval may be more disruptive for the lifestyles of certain subjects) and other clinical parameters (e.g., genotypes of the individual (e.g., HLA status (Bei et al. Nat Genet. 2010; 42:599-603; Hildesheim et al. J Natl Cancer Inst. 2002; 94:1780-9.), family history of NPC, dietary history, ethnic origin (e.g., Cantonese)).

In some cases, the methods provided herein comprise: receiving data from a first assay that comprises determining a characteristic of cell-free nucleic acid molecules from a pathogen in a biological sample of the subject, wherein the characteristic of the cell-free nucleic acid molecules from the pathogen comprises amount (e.g., copy number or percentage), methylation status, variant pattern, fragment size, coordinates of fragment ends, sequence motif of fragment ends or relative abundance as compared to cell-free nucleic acid molecules from the subject in the biological sample; and generating a report indicative of a risk for the subject to develop the pathogen-associated disorder based on the characteristic of the cell-free nucleic acid molecules from the pathogen and one or more factors of: age of the subject, smoking habit of the subject, family history of the pathogen-associated disorder of the subject, genotypic factors of the subject, or dietary history of the subject.

In aspects, provided herein are methods and systems for analyzing nucleic acid molecules in a biological sample from a subject. Examples of the methods and systems can involve analysis of variant pattern of nucleic acid molecules from a pathogen in the biological sample. In some cases, the nucleic acid molecules from the pathogen in the biological sample include cell-free nucleic acid molecules. Variant pattern analysis can involve comparison of the sequence of the nucleic acid molecules in a biological sample that are identified as originating from a pathogen with one or more reference genomes of the pathogen and subsequent determination of nucleotide variant pattern in the nucleic acid molecules from the pathogen in the biological sample.

In some cases, the methods and systems provided herein include determination of a status of or a risk for a pathogen-associated disorder in the subject based on the variant pattern in the nucleic acid molecules from the pathogen in the biological sample. For instance, the genetic variation of the EBV genome detected in the plasma can be used for the prediction of the risk of future NPC development. While it has previously been reported that the strains of EBV present in EBV-associated tumor and control samples (Palser et al. J Virol 2015; 89:5222-37) could be different, the tumor and control samples in this study were collected from different geographical locations. Given the geographical variations of EBV variants, it is therefore difficult to conclude whether the identified variants in tumor samples are geographically associated or disease-associated.

In some cases, the variant pattern analysis as described herein involves genomewide comparison between the nucleic acid molecules from the pathogen in the biological sample and one or more reference genomes of the pathogen. The genomewide comparison can involve sequence alignment across the whole genome of the pathogen and subsequent clustering analysis of the nucleotide variation pattern. In some cases, the genomewide comparison involves analysis of nucleotide variants at a large number of sites across the reference genome of the pathogen. These sites can include all sites across the whole genome of the pathogen. Alternatively, these sites across the reference genome of the pathogen, or variant sites, can include at least 30, at least 40, at least 50, at least 60, at least 70, at least 80, at least 90, at least 100, at least 200, at least 300, at least 400, at least 500, at least 600, at least 700, at least 800, at least 900, at least 1000, at least 1100, at least 1200, at least 1300, at least 1400, at least 1500, at least 1600, at least 1700, at least 1800, at least 1900, at least 2000, at least 3000, at least 4000, or at least 5000 sites at which nucleotide variations can typically be found. Nucleotide variants as described herein can include single nucleotide variants (SNVs). The variant sites used for variant pattern analysis as provided herein can include typical SNVs identified in the genome of the pathogen. In some cases, the variant sites can include insertions, deletions and fusions.

Genomewide variant pattern analysis provided herein can be superior to analysis of individual single nucleotide polymorphisms (SNPs). In an exemplary case, while SNPs on a fixed number of sites can be associated with particular strain(s) or subtype(s) of the pathogen that can lead to pathology in a subject, risk evaluation based on analysis of these individual SNPs can be limited to the particular strain(s) or subtype(s) of the pathogen and can fall in short in providing accurate assessment of the risk if other disease-rendering strain(s) or subtype(s) of the pathogen exist. In another exemplary case, genomewide variant pattern analysis provided herein can be beneficial when pathogen nucleic acid molecules in the biological sample are scarce, for instance, when cell-free nucleic acid molecules in biological samples such as plasma are analyzed. The available pathogen nucleic acid molecules in the biological sample may not have significant amount of coverage of the pathogen genome. As a result, genome wide variant pattern analysis that involves a large number of variant sites across the whole genome of the pathogen can provide a relatively more comprehensive readout of the genotypic feature of the cell-free nucleic acid molecules from the pathogen in the biological sample, whereas analyses involving a fixed number of individual polymorphisms are limited to a relatively small region or a number of small regions of the genome and thus can provide a relatively limited readout of the genotypic feature of the cell-free nucleic acid molecules from the pathogen in the biological sample.

In some cases, the variant pattern analysis provided herein include block-based pattern analysis, which involves segregating a reference genome of the pathogen into a plurality of bins and analyzing sequence reads relative to each of the plurality of bins. In some cases, the methods include determining a similarity index for each of the plurality of bins against the disorder-related reference genome of the pathogen. The similarity index can correlate with a proportion of the variant sites, within the respective bin, at which at least one of the sequence reads mapped to the reference genome of the pathogen has a same nucleotide variant as the disorder-related reference genome of the pathogen. In some cases, the disorder-related reference genome of the pathogen includes a plurality of disorder-related reference genomes of the pathogen, the methods include determining a respective similarity index for each of the plurality of bins against each of the plurality of disorder-related reference genomes of the pathogen; and determining a bin score for each of the plurality of bins based on a proportion of the plurality of disorder-related reference genomes, against which the respective similarity index within the respective bin is above a cutoff value.

### Assay of Cell-Free Nucleic Acid Molecules

The screening assay of the cell-free nucleic acid molecules from a biological sample of the subject can be any appropriate nucleic acid assays. For example, sequencing methods can be employed for analyzing the amount (e.g., copy number or percentage), methylation status, fragment size or relative abundance of the cell-free nucleic acid molecules. Alternatively or additionally, amplification or hybridization-based methods can also be used, such as, various polymerase chain reaction (PCR) methods, or microarray-based approaches. In some cases, immunoprecipitation methods are used, for instance, for analyzing methylation status of the nucleic acid molecules.

In some examples of the present disclosure, the screening assay to detect the cell-free pathogen nucleic acid molecules, e.g., cell-free EBV DNA, includes more than one test performed at different time points, and the detectability of the cell-free pathogen nucleic acid molecules over the multiple tests can be indicative of the risk for the subject to develop the pathogen-associate disorder. For example, the assay can include a two-step assay, or an assay regimen that includes 3, 4, 5, 6, 7, 8, 9, 10, or even more tests. Some of the tests can be performed at a same time point, while others at different time point(s), alternatively, all the tests can be performed at different time points.

The timing of the different screening assays, or the screening frequency can be determined by the methods and systems provided herein. The interval between the first screening assay and the second screening assay can be at least about 2 months, 4 months, 6 months, 8 months, 10 months, or 12 months. In some cases, the interval is at least about 12 months. The interval between the first screening assay and the second screening assay can be about 1 year, 1.5 years, 2 years, 2.5 years, 3 years, 3.5 years, 4 years, 4.5 years, 5 years, 6 years, 7 years, 8 years, 9 years, 10 years, or more. The interval can be long as the subject is normally diagnosed as not having the pathogen-associated disorder by well-established clinical diagnostic method (e.g., having no clinically detectable pathogen-associated disorder), even though the first screening assay can give a positive result indicating the presence of the pathogen-associated disorder. The methods and systems provided herein can enable prediction of the risk for the subject to develop the pathogen-associated disorder in the future, such as, within 6 months, 12 months, 2 years, 3 years, 5 years, or 10 years. Based on the evaluated risk, an appropriate follow-up time point can be determined.

The time between obtaining a sample and performing an assay can be optimized to improve the sensitivity and/or specificity of the assay or method. In some embodiments, a sample can be obtained immediately before performing an assay (e.g., a first sample is obtained prior to performing the first assay, and a second sample is obtained after performing the first assay but prior to performing the second assay). In some embodiments, a sample can be obtained, and stored for a period of time (e.g., hours, days or weeks) before performing an assay. In some embodiments, an assay can be performed on a sample within 1 day, 2 days, 3 days, 4 days, 5 days, 6 days, 1 week, 2 weeks, 3 weeks, 4 weeks, 5 weeks, 6 weeks, 7 weeks, 8 weeks, 3 months, 4 months, 5 months, 6 months, 1 year, or more than 1 year after obtaining the sample from the subject.

The time between performing an assay (e.g., a first assay or a second assay) and determining if the sample includes a marker or a set of markers indicative of the disorder, e.g., tumor, can vary. In some instances, the time can be optimized to improve the sensitivity and/or specificity of the assay or method. In some embodiments, determining if the sample includes a marker or a set of markers indicative of a tumor can occur within at most 0.1 hour, 0.5 hours, 1 hour, 2 hours, 4 hours, 8 hours, 12 hours, 24 hours, 2 days, 3 days, 4 days, 5 days, 6 days, 1 week, 2 weeks, 3 weeks, or 1 month of performing the assay.

Sequencing analysis of a biological sample as described herein can be performed for analysis of the one or more characteristics of the cell-free nucleic acid molecules from a pathogen. Methods provided herein can include sequencing nucleic acid molecules, e.g., cell-free nucleic acid molecules, cellular nucleic acid molecules, or both, from a biological sample. In some instances, methods provided herein include analyzing sequencing results, e.g., sequencing reads, from nucleic acid molecules from a biological sample. Methods and systems provided herein can involve or not involve an active step of sequencing. Methods and systems can include or provide means for receiving and processing sequencing data from a sequencer. Methods and systems can also include or provide means for providing commands to sequencer to adjust parameter(s) of sequencing process, e.g., commands based on the analysis of the sequencing results.

Commercially available sequencing equipment can be used for methods provided in the present disclosure, such as Illumina sequencing platform and the 454/Roche platform. Sequencing the nucleic acid can be performed using any method known in the art. For example, sequencing can include next generation sequencing. In some instances, sequencing the nucleic acid can be performed using chain termination sequencing, hybridization sequencing, Illumina sequencing (e.g., using reversible terminator dyes), ion torrent semiconductor sequencing, mass spectrophotometry sequencing, massively parallel signature sequencing (MPSS), Maxam-Gilbert sequencing, nanopore sequencing, polony sequencing, pyrosequencing, shotgun sequencing, single molecule real time (SMRT) sequencing, SOLiD sequencing (hybridization using four fluorescently labeled di-base probes), universal sequencing, or any combination thereof.

One sequencing method that can be used in the methods as provided herein can involve paired end sequencing, e.g., using an Illumina “Paired End Module” with its Genome Analyzer. Using this module, after the Genome Analyzer has completed the first sequencing read, the Paired-End Module can direct the resynthesis of the original templates and the second round of cluster generation. By using paired end reads in the methods provided herein, one can obtain sequence information from both ends of the nucleic acid molecules and map both ends to a reference genome, e.g., a genome of a pathogen or a genome of a host organism. After mapping both ends, one can determine a pathogen integration profile according to some embodiments of the methods as provided herein.

During paired-end sequencing, the sequence reads from a first end of the nucleic acid molecule can include at least 20, at least 25, at least 30, at least 35, at least 40, at least 45, at least 50, at least 55, at least 60, at least 65, at least 70, at least 75, at least 80, at least 85, at least 90, at least 95, at least 100, at least 105, at least 110, at least 105, at least 120, at least 125, at least 130, at least 135, at least 140, at least 145, at least 150, at least 155, at least 160, at least 165, at least 170, at least 175, or at least 180 consecutive nucleotides. The sequence reads from a first end of the nucleic acid molecule can include at most 24, at most 28, at most 32, at most 38, at most 42, at most 48, at most 52, at most 58, at most 62, at most 68, at most 72, at most 78, at most 82, at most 88, at most 92, at most 98, at most 102, at most 108, at most 122, at most 128, at most 132, at most 138, at most 142, at most 148, at most 152, at most 158, at most 162, at most 168, at most 172, or at most 180 consecutive nucleotides. The sequence reads from a first end of the nucleic acid molecule can include about 20, about 25, about 30, about 35, about 40, about 45, about 50, about 55, about 60, about 65, about 70, about 75, about 80, about 85, about 90, about 95, about 100, about 105, about 110, about 105, about 120, about 125, about 130, about 135, about 140, about 145, about 150, about 155, about 160, about 165, about 170, about 175, or about 180 consecutive nucleotides. The sequence reads from a second end of the nucleic acid molecule can include at least 20, at least 25, at least 30, at least 35, at least 40, at least 45, at least 50, at least 55, at least 60, at least 65, at least 70, at least 75, at least 80, at least 85, at least 90, at least 95, at least 100, at least 105, at least 110, at least 105, at least 120, at least 125, at least 130, at least 135, at least 140, at least 145, at least 150, at least 155, at least 160, at least 165, at least 170, at least 175, or at least 180 consecutive nucleotides. The sequence reads from a second end of the nucleic acid molecule can include at most 24, at most 28, at most 32, at most 38, at most 42, at most 48, at most 52, at most 58, at most 62, at most 68, at most 72, at most 78, at most 82, at most 88, at most 92, at most 98, at most 102, at most 108, at most 122, at most 128, at most 132, at most 138, at most 142, at most 148, at most 152, at most 158, at most 162, at most 168, at most 172, or at most 180 consecutive nucleotides. The sequence reads from a second end of the nucleic acid molecule can include about 20, about 25, about 30, about 35, about 40, about 45, about 50, about 55, about 60, about 65, about 70, about 75, about 80, about 85, about 90, about 95, about 100, about 105, about 110, about 105, about 120, about 125, about 130, about 135, about 140, about 145, about 150, about 155, about 160, about 165, about 170, about 175, or about 180 consecutive nucleotides. In some cases, the sequence reads from a first end of the nucleic acid molecule can include at least 75 consecutive nucleotides. In some cases, the sequence reads from a second end of the nucleic acid molecule can include at least 75 consecutive nucleotides. The sequence reads from a first end and a second end of a nucleic acid molecule can be of the same length or different lengths. The sequence reads from a plurality of nucleic acid molecules from a biological sample can be of the same length or different lengths.

Sequencing in the methods provided herein can be performed at various sequencing depth. Sequencing depth can refer to the number of times a locus is covered by a sequence read aligned to the locus. The locus can be as small as a nucleotide, or as large as a chromosome arm, or as large as the entire genome. Sequencing depth in the methods provided herein can be 50×, 100×, etc., where the number before “x” refers to the number of times a locus is covered with a sequence read. Sequencing depth can also be applied to multiple loci, or the whole genome, in which case x can refer to the mean number of times the loci or the haploid genome, or the whole genome, respectively, is sequenced. In some cases, ultra-deep sequencing is performed in the methods described herein, which can refer to performing at least 100× sequencing depth.

The number or the average number of times that a particular nucleotide within the nucleic acid is read during the sequencing process (e.g., the sequencing depth) can be multiple times larger than the length of the nucleic acid being sequenced. In some instances, when the sequencing depth is sufficiently larger (e.g., by at least a factor of 5) than the length of the nucleic acid, the sequencing can be referred to as ‘deep sequencing’. In some examples, the sequencing depth can be on average at least about 5 times greater, at least about 10 times greater, at least about 20 times greater, at least about 30 times greater, at least about 40 times greater, at least about 50 times greater, at least about 60 times greater, at least about 70 times greater, at least about 80 times greater, at least about 90 times greater, at least about 100 times greater than the length of the nucleic acid being sequenced. In some cases, the sample can be enriched for a particular analyte (e.g., a nucleic acid fragment, or a cancer-specific nucleic acid fragment).

A sequence read (or sequencing reads) generated in methods provided herein can refer to a string of nucleotides sequenced from any part or all of a nucleic acid molecule. For example, a sequence read can be a short string of nucleotides (e.g., 20-150) complementary to a nucleic acid fragment, a string of nucleotides complementary to an end of a nucleic acid fragment, or a string of nucleotides complementary to an entire nucleic acid fragment that exists in the biological sample. A sequence read can be obtained in a variety of ways, e.g., using sequencing techniques

### Amount/Detectability

One of the characteristics of the cell-free nucleic acid molecules that can be used in the methods and systems is amount (e.g., copy number or percentage) of the cell-free nucleic acid molecules from the pathogen. Some aspects of the present disclosure relate to stratification of the risk for a subject to develop the pathogen-associated disorder base on assessment of the amount (e.g., copy number or percentage) of the cell-free nucleic acid molecules from the pathogen in a biological sample from the subject.

Copy number of nucleic acid molecules in a biological sample can relate to the detectability of the nucleic acid molecules. Given a particular assay method, the detectability of the nucleic acid template can correlate to the copy number of the template molecules, e.g., a copy number that is below the lower detection limit of the assay method can be undetectable, while a copy number that is equal to or above the lower detection limit of the assay method can be termed as “detectable.” For instance, quantitative polymerase chain reaction (qPCR) method normally can have a detection limit, under which the signals of template molecules cannot be distinguished from background noise. Thus, in some cases, the methods and systems provided herein rely directly on the detectability of the cell-free nucleic acid molecules in the biological sample, which can correlate with their copy number in the biological sample. In some cases, the copy number of the cell-free nucleic acid molecules in the biological sample is directly measured. In other cases, the copy number is implicitly measured or inferred via detection of the cell-free nucleic acid molecules themselves.

Detection assays, such as, polymerase chain reaction (PCR) or quantitative PCR (qPCR), can be performed to assess the presence or absence or the copy number of cell-free nucleic acid molecules from a pathogen in a biological sample. Probes can be designed to target pathogen-specific genomic regions, for instance, EBV-specific genomic DNA sequence, human papillomavirus (HPV)-specific genomic DNA sequence, or hepatitis B virus (HBV)-specific genomic DNA sequence.

While examples and embodiments have been provided herein, additional techniques and embodiments related to, e.g., copy number and NPC, can be found in PCT AU/2011/001562, filed Nov. 30, 2011, which is incorporated herein by reference in its entirety. NPC can be closely associated with EBV infection. In southern China, the EBV genome can be found in the tumor tissues in almost all NPC patients. The plasma EBV DNA derived from NPC tissues has been developed as a tumor marker for NPC (Lo et al. Cancer Res 1999; 59: 1188-1191). In particular, a real-time qPCR assay can be used for plasma EBV DNA analysis targeting the BamHI-W fragment of the EBV genome. There can be about six to twelve repeats of the BamHI-W fragments in each EBV genome 5 and there can be approximately 50 EBV genomes in each NPC tumor cell (Longnecker et al. Fields Virology, 5th Edition, Chapter 61 “Epstein-Barr virus”; Tierney et al. J Virol. 2011; 85: 12362-12375). In other words, there can be on the order of 300-600 (e.g., about 500) copies of the PCR target in each NPC tumor cell. This high number of target per tumor cell can explain why plasma EBV DNA is a highly sensitive marker in the detection of early NPC. NPC cells can deposit fragments of the EBV DNA into the bloodstream of a subject. This tumor marker can be useful for the monitoring (Lo et al. Cancer Res 1999; 59: 5452-5455) and prognostication (Lo et al. Cancer Res 2000; 60: 6878-6881) of NPC.

A qPCR assay can also be used in a way similar to that described herein for EBV to measure amount of HPV, HBV, or any other viral DNA in a sample. Such analysis can be especially useful for screening of cervical cancer (CC), head and neck squamous cell carcinoma (HNSCC), hepatic cirrhosis, or hepatocellular carcinoma (HCC). In one example, the qPCR assay targets a region (e.g., 200 nucleotides) within the polymorphic L1 region of the HPV genome. More specifically, contemplated herein is the use of qPCR primers that selectively hybridize to sequences that encode one or more hypervariable surface loops in the L1 region.

Alternatively, the cell-free nucleic acid molecules from the pathogen can be detected and quantified using sequencing techniques. For example, cfDNA fragments can be sequenced and aligned to the HPV reference genome and quantified. Or in other examples, the sequence reads of cfDNA fragments are aligned to the reference genome of EBV or HBV and quantified.

The detectability or copy number of the cell-free nucleic acid molecules from the pathogen as measured by the assay provided herein can be indicative of the risk for the subject to develop the pathogen-associated disorders. In some examples, the higher the copy number of the cell-free nucleic acid molecules from the pathogen is, the higher risk the subject is disposed to develop the pathogen-associated disorders. In some cases, the detectability of the cell-free nucleic acid molecules from the pathogen over one or more assays over one particular time point or multiple time points is indicative of the risk for the subject to the develop the pathogen-associated disorders. The subject can be disposed to a higher risk for the pathogen-associated disorder when the cell-free nucleic molecules from the pathogen in a biological sample from the subject is detectable as compared when the molecules are not detectable by the assay provide herein. The multi-step detection assay can be performed at timing as discussed above.

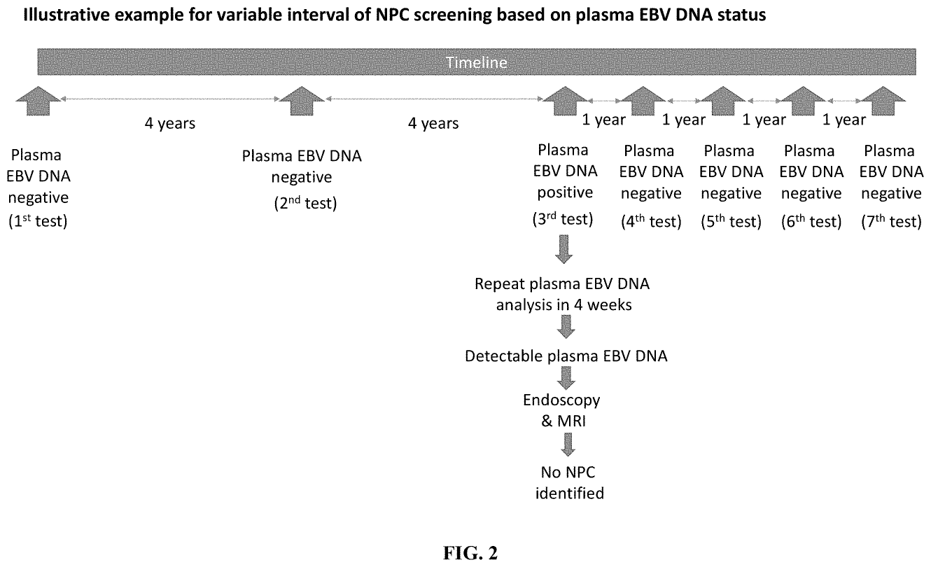

In some examples of the present disclosure, a two-step assay is performed to detect cell-free pathogen nucleic acid molecules in the biological sample. In some cases, a first test of the two-step assay is performed, and later a second test of the two-step assay is performed or not performed, depending on the assay result at the first time point. For instance, a second test of the two-step detection assay can be performed if the first test provides a positive result, e.g., cell-free pathogen nucleic acid molecules are detected in the first biological sample; the second test may not be performed if a negative result is obtained from the first test. In other cases, the second test is performed regardless of the first test. In some examples, the cases in which both tests of the two-step detection assay have positive result are termed as permanently positive, while the cases in which only the first or the second tests have positive result are termed as transiently positive. In one illustrative example, “positive” assay results are indicative of a higher risk for the subject to develop the pathogen-associated disorder, e.g., EBV-associated NPC, as compared to “negative” assay results, while a “permanently positive” assay result is indicative of a higher risk as compared to a “transiently positive” assay result. In some illustrative examples, a longer interval can be set between the first time point and the second time point when a permanent positive result is obtained out of the two-step detection assay performed at the first time point as compared to when a transiently positive result is obtained. For example, in an EBV-associated NPC screening, if a permanently positive result is obtained from a first two-step detection assay, a follow-up second screening assay can be recommended to be performed within about one year of the first detection assay. In contrast, if a transiently positive result is obtained from the first two-step detection assay, a follow-up second screening assay can be performed within about two years of the first detection assay. Four years or even longer interval can be placed for the follow-up screening assay if a negative result is obtained. In some cases, the preceding positive result indicative of a higher risk can override the interval selection that would be disposed by a subsequent result indicative of a lower risk. For example, in year 1 a permanently positive result is obtained, then the subject will be followed up every year for the following 4 years, regardless of the results obtained from the follow-up assays performed during the following 4 years. An illustrative example is given in FIG. 2 and described in more details in Example 2. Similar to the detection assay, risk evaluation based on other characteristic of the cell-free nucleic acid molecules from the pathogen can also follow this exemplary or similar screening regimen.

A second test of the assay can be performed hours, days, or weeks after the first assay. In one example, a second assay can be performed immediately after the first assay. In other cases, a second assay can be performed within 1 day, 2 days, 3 days, 4 days, 5 days, 6 days, 1 week, 2 weeks, 3 weeks, 4 weeks, 5 weeks, 6 weeks, 7 weeks, 8 weeks, 3 months, 4 months, 5 months, 6 months, 1 year, or more than 1 year after the first assay. In a particular example, the second assay can be performed within 2 weeks of the first sample. Generally, a second test of the assay can be used to improve the specificity with which a pathogen-associated disorder, e.g., tumor, can be detected in a patient. The time between performing the first test and the second test can be determined experimentally. In some embodiments, the method can include 2 or more tests, and both tests use the same sample (e.g., a single sample is obtained from a subject, e.g., a patient, prior to performing the first assay, and is preserved for a period of time until performing the second assay). For example, two tubes of blood can be obtained from a subject at the same time. A first tube can be used for a first test. The second tube can be used only if results from the first test from the subject are positive. The sample can be preserved using any method known to a person having skill in the art (e.g., cryogenically). This preservation can be beneficial in certain situations, for example, in which a subject can receive a positive test result (e.g., the first assay is indicative of cancer), and the patient can rather not wait until performing the second assay, opting rather to seek a second opinion.

### Methylation Status

Some aspects of the present disclosure relate to stratification of the risk for a subject to develop the pathogen-associated disorder based on assessment of the methylation status of the cell-free nucleic acid molecules from the pathogen in a biological sample from the subject.

Methylation of cell-free pathogen nucleic acid molecules can differentiate samples from patients having the pathogen-associated disorder (e.g., EBV-associated NPC or HPV-associated cervical cancer) and subjects without the disorder (e.g., non-NPC subjects). For instance, methylation status of plasma EBV DNA associated with NPC can be different from the methylation status of plasma EBV DNA detected in non-NPC subjects, as shown in U.S. patent application Ser. No. 16/046,795, which is incorporated herein by reference in its entirety. There can be regions with differential methylation between plasma DNA from NPC patients and non-NPC subjects with detectable EBV DNA when analyzed by bisulfite sequencing. As a result, analysis of methylation status at these differentially methylated regions can differentiate NPC and non-NPC subjects. As described herein, the NPC-associated EBV DNA methylation status can also predict the risk of NPC development and can be used for adjusting the interval of NPC screening. For example, subjects with NPC-associated EBV DNA methylation patterns can be screened more frequently compared with those without NPC-associated EBV DNA methylation patterns. In some cases, instead of bisulfite sequencing, another type of methylation-aware sequencing can be done, for example, using single molecule sequencing systems such as that from Pacific Biosciences (Kelleher et al. Methods Mol Biol. 2018; 1681:127-137; Powers et al. BMC Genomics. 2013; 14:675) and Oxford Nanopore (Simpson et al. Nat Methods. 2017; 14:407-10), as well as the use of methylation-sensitive restriction enzyme treatment prior to sequencing. In yet another case, one can use molecular approaches that are methylation aware and which are not sequencing based, e.g., methylation-specific PCR (Herman et al. Proc Natl Acad Sci USA. 1996; 93:9821-6), detection systems based on methylation-sensitive enzymes (e.g., restriction enzymes) and bisulfite conversion followed by mass spectrometry (van den Boom et al. Methods Mol Biol. 2009; 507:207-27; Nygren et al. Clin Chem. 2010; 56:1627-35), and approaches based on the differential precipitation of DNA molecules based on their methylation status (e.g., using anti-methylated cytosine antibody (Shen et al. Nature. 2018; 563:579-83; Zhou et al. PLoS One. 2018; 13:e0201586) or methylation-binding proteins (Zhang et al. Nat Commun. 2013; 4:1517).

In some cases, the methylation pattern of cell-free pathogen nucleic acid molecules, e.g., plasma EBV DNA, can be used for the detection of pathogen-associated disorders, e.g., pathogen-associated cancer, e.g., NPC, or the prediction of future risk of having clinically detectable disorder. As described above, one approach is to use bisulfite to treat the nucleic acid molecules for conversion of unmethylated cytosine into uracil. Methylated cytosine would not be altered by bisulfite and remains as cytosine. Subsequent examination of the bisulfite-treated nucleic acid molecules, such as sequencing, can be employed to detect the methylation status of the nucleic acid molecules in the biological sample.

In one example, the difference in the methylation level of plasma EBV DNA is determined using methylation-sensitive restriction enzyme analysis. One non-limiting example of methylation-sensitive restriction enzyme is HpaII which can cleave molecules carrying unmethylated “CCGG” motifs but leaves the molecules without “CCGG” or with methylated “CCGG” unchanged. Alternatively or additionally, other methylation-sensitive restriction enzymes can be used. In one example, because of the lower methylation level of plasma EBV DNA in non-cancer subjects, the plasma EBV DNA in non-cancer subjects can be more susceptible to the cutting by methylation-sensitive restriction enzymes. The susceptible of enzyme digestion can be determined, for example but not limited to massively parallel sequencing, gel electrophoresis, capillary electrophoresis, polymerase chain reaction (PCR), and real-time PCR.

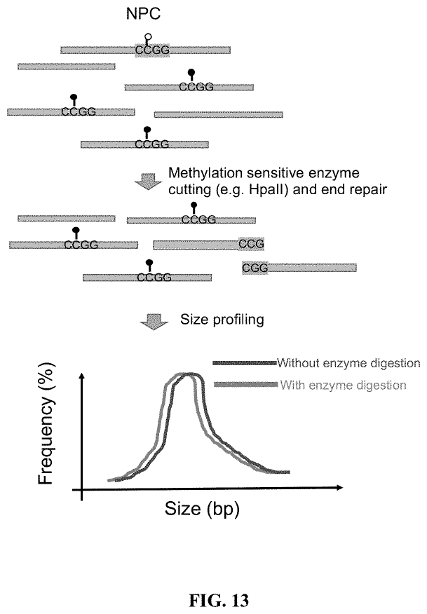

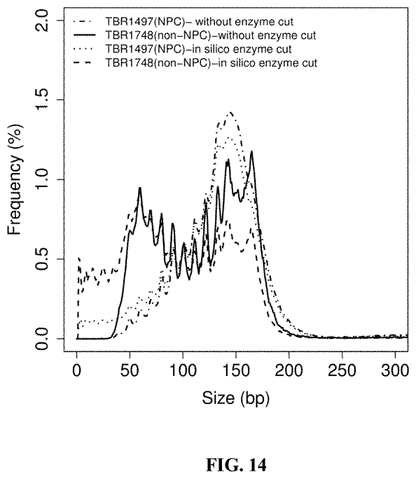

In the cases where sequencing, such as massively parallel sequencing, is used to analyze the degree of digestion by methylation-sensitive restriction enzyme, the size distribution of the pathogen cell-free nucleic acid molecules, e.g., plasma EBV DNA, with and without enzyme digestion, can be used to reflect the degree of digestion. As shown in FIGS. 12 and 13, shift of the size distribution curve to the left can indicate the shortening of the size distribution of the plasma EBV DNA. The more the curve is shift to the left can reflect a higher degree of enzyme digestion and imply the lower methylation level of DNA.

The methylation status of the cell-free pathogen nucleic acid molecules as described herein can include methylation density for individual methylation sites, a distribution of methylated/unmethylated sites over a contiguous region on the genome of the pathogen, a pattern or level of methylation for each individual methylation site within one or more particular regions on the genome of the pathogen or across the whole genome of the pathogen, and non-CpG methylation. In some cases, the methylation status includes methylation level (or methylation density) for individual differentiated methylation sites that can be identified between, for instance, samples from patients having the pathogen-associated disorder (e.g., EBV-associated NPC or HPV-associated cervical cancer) and subjects without the disorder (e.g., non-NPC subjects). The methylation density can refer to, for a given methylation site, a fraction of nucleic acid molecules methylated at the given methylation site over the total number of nucleic acid molecules of interest that contain such methylation site. For instance, the methylation density of a first methylation site in liver tissue can refer to a fraction of liver DNA molecules methylated at the first site over the total liver DNA molecules. In some cases, the methylation status includes coherence (e.g., pattern or haplotype) of methylation/unmethylation status among individual methylation sites.

In some cases, a screening assay as described herein (e.g., first assay or a second assay) can include determining a methylation status of the cell-free nucleic acid molecules by any technique available, such as, but not limited to, performing methylation-aware sequencing, methylation-sensitive amplification, or methylation-sensitive precipitation. While examples and embodiments have been provided herein, additional techniques and embodiments related to, e.g., determining a methylation status, can be found in PCT AU/2013/001088, filed Sep. 20, 2013, which is entirely incorporated herein by reference.

### Fragment Size

Some aspects of the present disclosure relate to stratification of the risk for a subject to develop the pathogen-associated disorder base on assessment of the fragment size of the cell-free nucleic acid molecules from the pathogen in a biological sample from the subject.

Fragment size distribution and/or relative abundance of cell-free pathogen nucleic acid molecules can differentiate samples from patients having the pathogen-associated disorder (e.g., EBV-associated NPC or HPV-associated cervical cancer) and subjects without the disorder (e.g., non-NPC subjects). For instance, the size distribution of plasma EBV DNA molecules and the ratio of circulating DNA molecules mapping to the EBV genome and the human genome can be useful for differentiating NPC patients from non-NPC subjects with detectable plasma EBV DNA, as demonstrated using massive parallel sequencing in Lam et al. Proc Natl Acad Sci USA. 2018; 115:E5115-E5124, which is incorporated herein by reference in its entirety. According to some examples of the present disclosure, the NPC-associated size distribution and relative abundance of circulating DNA mapping to the EBV and human genome can also be useful for the prediction of the risk of developing future, clinically detectable NPC. In one implementation, subjects with these NPC-associated features on plasma DNA sequencing but without a detectable NPC can be followed up more frequently than those with detectable plasma EBV DNA but without these NPC-associated features. One potential practical advantage of using this sequencing-based analysis to stratify the risk of NPC over using the two-step assay as discussed above can be that the collection of another blood sample from the patient can be omitted.

In some cases, an assay (e.g., first assay or a second assay) can include performing an assay, e.g., next generation sequencing assay, to analyze nucleic acid fragment size, e.g., fragment size of plasma EBV DNA. In some cases, sequencing is used to assess size of cell-free viral nucleic acids in a sample. For example, the size of each sequenced plasma DNA molecule can be derived from the start and end coordinates of the sequence, where the coordinates can be determined by mapping (aligning) sequence reads to a viral genome. In various examples, the start and end coordinates of a DNA molecule can be determined from two paired-end reads or a single read that covers both ends, as may be achieved in single-molecule sequencing. In some cases, amplification or hybridization-based methods can also be used for fragment size analysis. For instance, probes can be designed to target genomic regions of various lengths, amplification (e.g., PCR or qPCR) or hybridization signal can indicate the number of cell-free nucleic acid fragments at the target genomic region while having a length equal to or larger than the target region. The fragment size distribution can thus be deduced. Methods for the fragment size assay and analyses can include the ones described in U.S. patent publication number US20180208999A1, which is incorporated herein by reference in its entirety.

A fragment size distribution can be displayed as a histogram with the size of a nucleic acid fragment on the horizontal axis. The number of nucleic acid fragments at each size (e.g., within 1 bp resolution) can be determined and plotted on the vertical axis, e.g., as a raw number or frequency percentage. The resolution of size can be more than 1 bp (e.g., 2, 3, 4, or 5 bp resolution). The following analysis of size distributions (also referred to as size profiles) shows that the viral DNA fragments in a cell-free mixture from NPC subjects are statistically longer than in subjects with no observable pathology. In one illustrative example, in a fragment size distribution curve obtained from plasma EBV DNA analysis, there can be a characteristic 166-bp peak (nucleosomal pattern) in the plasma EBV DNA size profile of NPC patients, while plasma EBV DNA from non-cancer subjects do not exhibit the typical nucleosomal pattern.

In some cases, the relative abundance of the cell-free nucleic acid molecules from the pathogen as compared to the cell-free nucleic acid molecules from the subject is calculated for evaluating the risk. In some cases, the relative abundance is analyzed in terms of a size ratio. In various examples, the size ratio of pathogen fragments versus cell-free fragments from the subject refers to amount ratio between cell-free nucleic acid fragments from the pathogen and cell-free nucleic acid fragments from the subject. For example, a size ratio of EBV DNA fragments between 80 and 110 base pairs can be:

$${Size}_{80 \text{-} 110 bp} ratio = \frac{Proportion of EBV DNA fragments within 80 \text{-} 110 bp}{Proportion of autosomal DNA fragments within 80 \text{-} 110 bp}$$

In various cases, a cutoff value or a threshold is set for the evaluation. For instance, there can be a size threshold for determining a size ratio between the pathogen fragments and the subject autosomal fragments. Or in some cases, a size threshold is set so that a number of fragments having a size below or above the threshold is considered as indicative of a risk for the subject to develop the pathogen-associated disorder. It should be understood that the size threshold can be any value. The size threshold may be at least about 10 bp, 20 bp, 25 bp, 30 bp, 35 bp, 40 bp, 45 bp, 50 bp, 55 bp, 60 bp, 65 bp, 70 bp, 75 bp, 80 bp, 85 bp, 90 bp, 95 bp, 100 bp, 105 bp, 110 bp, 115 bp, 120 bp, 125 bp, 130 bp, 135 bp, 140 bp, 145 bp, 150 bp, 155 bp, 160 bp, 165 bp, 170 bp, 175 bp, 180 bp, 185 bp, 190 bp, 195 bp, 200 bp, 210 bp, 220 bp, 230 bp, 240 bp, 250 bp, or greater than 250 bp. For example, the size threshold can be 150 bp. In another example, the size threshold can be 180 bp. In some embodiments, an upper and a lower size threshold may be used (e.g., a range of values). In some embodiments, an upper and a lower size threshold may be used to select nucleic acid fragments having a length between the upper and lower cutoff values. In some embodiments, an upper and a lower cutoff may be used to select nucleic acid fragments having a length greater than the upper cutoff value and less than the lower size threshold. In some cases, a cutoff value for the size ratio is used to determine if a subject has a risk or how much the risk is for the subject to develop a pathogen-associated disorder, e.g., NPC. For example, subjects with NPC have a lower size ratio within the size range of 80 to 110 bp than subjects with false-positive plasma EBV DNA results. In some cases, a cutoff value for a size ratio can be about 0.1, about 0.5, about 1, about 2, about 3, about 4, about 5, about 6, about 7, about 8, about 9, about 10, about 11, about 12, about 13, about 14, about 15, about 16, about 17, about 18, about 19, about 20, about 25, about 50, about 100, or greater than about 100. In some cases, a cutoff value for a size index can be about or least 10, about or least 2, about or least 1, about or least 0.5, about or least 0.333, about or least 0.25, about or least 0.2, about or least 0.167, about or least 0.143, about or least 0.125, about or least 0.111, about or least 0.1, about or least 0.091, about or least 0.083, about or least 0.077, about or least 0.071, about or least 0.067, about or least 0.063, about or least 0.059, about or least 0.056, about or least 0.053, about or least 0.05, about or least 0.04, about or least 0.02, about or least 0.001, or less than about 0.001.

Various statistical values of a size distribution of nucleic acid fragments can be determined. For example, an average, mode, median, or mean of a size distribution can be used. Other statistical values can be used, e.g., a cumulative frequency for a given size or various ratios of amount of nucleic acid fragments of different sizes. A cumulative frequency can correspond to a proportion (e.g., a percentage) of DNA fragments that are of a given size or smaller, or larger than a given size. The statistical values provide information about the distribution of the sizes of nucleic acid fragments for comparison against one or more cutoffs for determining a level of pathology resulting from a pathogen. The cutoffs can be determined using cohorts of healthy subjects, subjects known to have one or more pathologies, subjects that are false positives for a pathology associated with the pathogen, and other subjects mentioned herein. One skilled in the art will know how to determine such cutoffs based on the description herein.

In some examples, the first statistical value of sizes of pathogen fragments can be compared to a reference statistical value of sizes from the human genome. For example, a separation value (e.g., a difference or ratio) can be determined between the first statistical value and a reference statistical value, e.g., determined from other regions in the pathogen reference genome or determined from the human nucleic acids. The separation value can be determined from other values as well. For example, the reference value can be determined from statistical values of multiple regions. The separation value can be compared to a size threshold to obtain a size classification (e.g., whether the DNA fragments are shorter, longer, or the same as a normal region).

Some examples can calculate a parameter (separation value), which can be defined as a difference in the proportion of short DNA fragments between the reference pathogen genome and the reference human genome using the following equation:

ΔF=P(≤150bp)ₜₑₛₜ−P(≤150bp)_{ref}

where P(≤150 bp)ₜₑₛₜ denotes the proportion of sequenced fragments originating from the tested test region with sizes ≤150 bp, and P(≤150 bp)_{ref} denotes the proportion of sequenced fragments originating from the reference region with sizes ≤150 bp. In other embodiments, other size thresholds can be used, for example but not limited to 100 bp, 110 bp, 120 bp, 130 bp, 140 bp, 160 bp and 166 bp. In other embodiments, the size thresholds can be expressed in bases, or nucleotides, or other units.

A size-based z-score can be calculated using the mean and SD values of control subjects.

$$Size \text{-} based z \text{-} score = \frac{Δ {F}_{sample} - mean Δ {F}_{control}}{S D Δ {F}_{control}}$$

In some embodiments, a size-based z-score of >3 indicates an increased proportion of short fragments for the pathogen, while a size-based z-score of <−3 indicates a reduced proportion of short fragments for the pathogen. Other size thresholds can be used. Further details of a size-based approach can be found in U.S. Pat. Nos. 8,620,593 and 8,741,811, and U.S. Patent Publication 2013/0237431, each of which is incorporated by reference in its entirety.

To determine a size of a nucleic acid fragment, at least some examples of the present disclosure can work with any single molecule analysis platform in which the chromosomal origin and the length of the molecule can be analyzed, e.g., electrophoresis, optical methods (e.g., optical mapping and its variants, en.wikipedia.org/wiki/Optical_mapping#cite_note-Nanocoding-3, and Jo et al. Proc Natl Acad Sci USA. 2007; 104: 2673-2678), fluorescence-based method, probe-based methods, digital PCR (microfluidics-based, or emulsion-based, e.g., BEAMing (Dressman et al. Proc Natl Acad Sci USA. 2003; 100: 8817-8822), RainDance (www.raindancetech.com/technology/per-genomics-research.asp)), rolling circle amplification, mass spectrometry, melting analysis (or melting curve analysis), molecular sieving, etc. As an example for mass spectrometry, a longer molecule would have a larger mass (an example of a size value).

In one example, nucleic acid molecules can be randomly sequenced using a paired-end sequencing protocol. The two reads at both ends can be mapped (aligned) to a reference genome, which may be repeat-masked (e.g., when aligned to a human genome). The size of the DNA molecule can be determined from the distance between the genomic positions to which the two reads mapped.

### Variant Pattern Analysis

Some aspects of the present disclosure relates to stratification of the risk for a subject to develop the pathogen-associated disorder base on assessment of the variant pattern of the cell-free nucleic acid molecules from the pathogen in a biological sample from the subject. Genetic variation of the pathogen genome detected in the biological sample can be used for the prediction of the risk of future development of the pathogen-associated disorder.

Variant pattern of pathogen nucleic acid molecules can be different in diseased tissue from patients having a pathogen-associated disorder (e.g., pathogen-associated malignant tumor) as compared to sample from subject without the pathogen-associated disorder. It has been reported that the strains of EBV present in EBV-associated tumor and control samples (Palser et al. J Virol. 2015; 89:5222-37) might be different. However, in this previous study, the tumor and control samples were collected from different geographical locations. Given the potential geographical variations of EBV variants, it can be difficult to conclude whether the identified variants in tumor samples are geographically associated or disease-associated. There were previous attempts to identify NPC-associated EBV variants through analysis of NPC tumor samples. In one genomewide association study (GWAS) (Hui et al. Int J Cancer 2019, doi.org/10.1002/ijc.32049) which analyzed NPC tumor and saliva samples from individuals with no EBV-associated diseases from the same geographical region, there were 29 polymorphisms (single nucleotide polymorphisms (SNP) or indels) identified below the false discovery rate with an adjusted P of 0.05. These 29 NPC-associated EBV variants were shown to be present in over 90% of NPC cases but only 40-50% of control cases.

In contrast to analysis of the individual EBV polymorphisms for developing NPC (Hui et al. Int J Cancer 2019, doi.org/10.1002/ijc.32049; Feng et al. Chin J Cancer 2015; 34:61), aspects of the present disclosure provide methods and systems for analysis of pathogen nucleic acid molecules for the variant pattern in a genomewide manner. Furthermore, rather than identification of disease-associated EBV variants through analysis of tumor and cell line samples (Palser et al. J Virol. 2015; 89:5222-37, Correia et al. J Virol. 2018; 92:e01132-18, Hui et al. Int J Cancer 2019, doi.org/10.1002/ijc.32049), aspects of the present disclosure provide methods and systems for analysis of pathogen variant patterns through analyzing cell-free pathogen nucleic acid molecules, such as in blood (e.g., plasma or serum), nasal flushing fluid, nasal brush sample, or other bodily fluids obtained via non-invasive or minimally invasive procedures as compared to invasive biopsy of tumors. In one illustrative example, the low abundance and also fragmented nature of EBV DNA molecules in blood can pose technical challenges to the analysis. Analysis of variant patterns of cell-free viral DNA molecules in a non-invasive manner can enhance the clinical applications including screening, predictive medicine, risk stratification, surveillance and prognostication. In one example, the analysis can be used to differentiate subjects with different virus-associated conditions, for example, NPC patients and non-NPC subjects with detectable plasma EBV DNA in the context of screening. In another example, it can be used for disease or cancer risk prediction.

Different approaches can be used to obtain a variant pattern. Non-limiting assay methods can include massively parallel sequencing (MPS), Sanger sequencing (such as that used in Lorenzetti et al. J Clin Microbiol. 2012; 50:609-18), and microarray-based SNP analysis (such as that described in Wang et al. PNAS 2002; 99:15687-92), hybridization analysis, and mass spectrometric analysis. In one illustrative example, sequencing method such as targeted sequencing with capture enrichment, MPS or Sanger Sequencing is used, and the sequence reads are analyzed with reference to a reference genome of the pathogen (e.g., EBV reference genome) on a per nucleotide basis. The method can include obtaining sequence reads of cell-free nucleic acid molecules from a biological sample of a subject. The method can further include aligning the sequence reads to a reference genome of the pathogen. The method can further include analyzing nucleotide variant pattern across the reference genome of the pathogen by analyzing the nucleotide variation between the reference genome of the pathogen and sequence reads mapped to the reference genome of the pathogen. The variant pattern as provided herein can characterize a nucleotide variant of the sequence reads mapped to the reference genome of the pathogen at each of a plurality of variant sites on the reference genome of the pathogen. The plurality of variant sites can include at least 30, at least 40, at least 50, at least 60, at least 70, at least 80, at least 90, at least 100, at least 200, at least 300, at least 400, at least 500, at least 600, at least 700, at least 800, at least 900, at least 1000, at least 1100, or at least 1200 sites across the reference genome of the pathogen. In some cases, the plurality of variant sites includes at least 1000 sites across the reference genome of the pathogen. In some cases, the plurality of variant sites includes about 1100 sites across the reference genome of the pathogen. In some cases, the plurality of variant sites includes at least 600 sites across the reference genome of the pathogen. In some cases, the plurality of variant sites includes about 660 sites across the reference genome of the pathogen. In some cases, the plurality of variant sites includes at least 30, 40, 50, 100, 150, 200, 250, 300, 350, 400, 450, 500, 550, or 600 sites selected from genomic sites as set forth in Table 6 relative to EBV reference genome (AJ507799.2). In some cases, the plurality of variant sites includes a genomic sites as set forth in Table 6 relative to EBV reference genome (AJ507799.2).

In some cases, the variant pattern of the cell-free nucleic acid molecules from the pathogen characterizes nucleotide variant of the sequence reads mapped to the referenced genome of the pathogen at each of the plurality of variant sites that are randomly selected from genomic sites as set forth in Table 6 relative to EBV reference genome (AJ507799.2). In some cases, the method provided herein comprises a step of randomly selecting a plurality of variant sites from genomic sites as set forth in Table 6 relative to EBV reference genome (AJ507799.2). The method can further comprise analyzing nucleotide variant pattern over the randomly selected plurality of variant sites by analyzing the nucleotide variation between the reference genome of the pathogen and sequence reads mapped to the reference genome of the pathogen.

In some cases, the variant pattern of the cell-free nucleic acid molecules from the pathogen characterizes nucleotide variant of the sequence reads mapped to the referenced genome of the pathogen at each of the plurality of variant sites that comprise at least 30, 40, 50, 100, 150, 200, 250, 300, 350, 400, 450, 500, 550, or 600 sites randomly selected from genomic sites as set forth in Table 6 relative to EBV reference genome (AJ507799.2).

In some cases, the plurality of variant sites consists of all sites at which the sequence reads mapped to the reference genome of the pathogen have a different nucleotide variant than the reference genome of the pathogen.

In some cases, a wild type pathogen genome is used as the reference genome. For instance, a wide type EBV genome (GenBank: AJ507799.2) can be used as the reference EBV genome. In other cases, other pathogen genome is used as the reference genome. In yet another example, multiple pathogen genomes (e.g., EBV genomes) are used as the reference. In yet another example, a consensus sequence is used as the reference. The consensus can be built by combining variants of different pathogen genomic sequences, for instance, the consensus sequence of EBV genome as described in de Jesus et al. J Gen Virol. 2003; 84:1443-50.

Sequence alignment utilized in the methods and systems provided herein, for instance, for analysis of copy number, methylation status, fragment size, relative abundance, or variant pattern, can be performed by any appropriate bioinformatics algorithms, programs, toolkits, or packages. For instance, one can use the short oligonucleotide analysis package (SOAP) as an alignment tool for applications of methods and systems as provided herein. Examples of short sequence reads analysis tools that can be used in the methods and systems provided herein include Arioc, BarraCUDA, BBMap, BFAST, BigBWA, BLASTN, BLAT, Bowtie, Bowtie2, BWA, BWA-PSSM, CASHX, Cloudburst, CUDA-EC, CUSHAW, CUSHAW2, CUSHAW2-GPU, CUSHAW3, drFAST, ELAND, ERNE, GASSST, GEM, Genalice MAP, Geneious Assembler, GensearchNGS, GMAP and GSNAP, GNUMAP, HIVE-hexagon, Isaac, LAST, MAQ, mrFAST, mrsFAST, MOM, MOSAIK, MPscan, Novoalign & NovoalignCS, NextGENe, NextGenMap, Omixon Variant Toolkit, PALMapper, Partek Flow, PASS, PerM, PRIMEX, QPalma, RazerS, REAL, cREAL, RMAP, rNA, RTG Investigator, Segemehl, SeqMap, Shrec, SHRiMP, SLIDER, SOAP, SOAP2, SOAP3, SOAP3-dp, SOCS, SparkBWA, SSAHA, SSAHA2, Stampy, SToRM, Subread, Subjunc, Taipan, UGENE, VelociMapper, XpressAlign, and ZOOM.

A number of consecutive nucleotides (“a sequence stretch”) in a sequence read can be used to align to a reference genome to make a call regarding alignment. For example, the alignment can include aligning at least 4, at least 6, at least 8, at least 10, at least 12, at least 14, at least 16, at least 18, at least 20, at least 22, at least 24, at least 25, at least 26, at least 28, at least 30, at least 32, at least 34, at least 35, at least 36, at least 38, at least 40, at least 42, at least 44, at least 45, at least 46, at least 48, at least 50, at least 52, at least 54, at least 55, at least 56, at least 58, at least 60, at least 62, at least 64, at least 65, at least 66, at least 67, at least 68, at least 69, at least 70, at least 71, at least 72, at least 73, at least 74, at least 75, at least 76, at least 78, at least 80, at least 82, at least 84, at least 85, at least 86, at least 88, at least 90, at least 92, at least 94, at least 95, at least 96, at least 98, at least 100, at least 102, at least 104, at least 106, at least 108, at least 110, at least 112, at least 114, at least 116, at least 118, at least 120, at least 122, at least 124, at least 126, at least 128, at least 130, at least 132, at least 134, at least 136, at least 138, at least 140, at least 142, at least 145, at least 146, at least 148, or at least 150 consecutive nucleotides of a sequence read to a reference genome, e.g., a reference genome of a pathogen, or a reference genome of a host organism. In some cases, alignment as mentioned herein can include aligning at most 5, at most 7, at most 9, at most 11, at most 13, at most 15, at most 17, at most 19, at most 21, at most 23, at most 25, at most 27, at most 29, at most 31, at most 33, at most 35, at most 37, at most 39, at most 41, at most 43, at most 45, at most 47, at most 49, at most 51, at most 53, at most 55, at most 57, at most 59, at most 61, at most 63, at most 65, at most 67, at most 68, at most 69, at most 70, at most 71, at most 72, at most 73, at most 74, at most 75, at most 76, at most 78, at most 80, at most 81, at most 83, at most 85, at most 87, at most 89, at most 91, at most 93, at most 95, at most 97, at most 99, at most 101, at most 103, at most 105, at most 107, at most 109, at most 111, at most 113, at most 115, at most 117, at most 119, at most 121, at most 123, at most 125, at most 127, at most 129, at most 131, at most 133, at most 135, at most 137, at most 139, at most 141, at most 143, at most 145, at most 147, at most 149, or at most 151 consecutive nucleotides of a sequence read to a reference genome, e.g., a reference genome of a pathogen, or a reference genome of a host organism. In some instances, alignment as mentioned herein includes aligning about 20, about 22, about 24, about 25, about 26, about 28, about 30, about 32, about 34, about 35, about 36, about 38, about 40, about 42, about 44, about 45, about 46, about 48, about 50, about 52, about 54, about 55, about 56, about 58, about 60, about 62, about 64, about 65, about 66, about 67, about 68, about 69, about 70, about 71, about 72, about 73, about 74, about 75, about 76, about 78, about 80, about 82, about 84, about 85, about 86, about 88, about 90, about 92, about 94, about 95, about 96, about 98, about 100, about 102, about 104, about 106, about 108, about 110, about 112, about 114, about 116, about 118, about 120, about 122, about 124, about 126, about 128, about 130, about 132, about 134, about 136, about 138, about 140, about 142, about 145, about 146, about 148, about 150, about 152, about 154, about 155, about 156, about 158, about 160, about 162, about 164, about 165, about 166, about 168, about 170, about 172, about 174, about 175, about 176, about 178, about 180, about 185, about 190, about 195, or about 200 consecutive nucleotides of a sequence read to a reference genome, e.g., a reference genome of a pathogen, or a reference genome of a host organism.

In some cases, an alignment call is made, when the sequence stretch has at least 80%, at least 85%, at least 90%, at least 95%, at least 98%, at 99%, or 100% sequence identity or complementarity to a particular region of a reference genome, e.g., a human reference genome, over the entire sequence read. In some cases, an alignment call is made when the sequence stretch has at least 80% sequence identity or complementarity to a particular region of a reference genome, e.g., a human reference genome, over the entire sequence read. In some cases, an alignment call is made when the sequence stretch is identical or complementary to a particular region of a reference genome, e.g., a human reference genome, with mismatches of no more than 20, 15, 10, 9, 8, 7, 6, 5, 4, 3, 2, or 1 bases, or with zero mismatches. In some cases, an alignment call is made when the sequence stretch is identical or complementary to a particular region of a reference genome, e.g., a human reference genome, with no more than mismatches of 2 bases. The maximum mismatch number or percentage, or the minimum similarity number or percentage can vary as a selection criterion depending on purposes and contexts of application of the methods and systems provided herein.

In some cases, the alignment of sequence reads to a reference genome of the pathogen allows a maximum mismatch of no more than 20, 15, 10, 9, 8, 7, 6, 5, 4, 3, 2, or 1 bases. The mismatch between the mapped sequence reads and the reference genome of the pathogen can indicate nucleotide variation in the pathogen genomic sequence present in the biological sample, in other cases, it can also indicate sequencing error. Without wishing to be bound by a certain theory, more than one nucleotide variant is identified at a given genomic site in one biological sample can be due to the sequencing error or heterogeneity of the diseased cells that the cell-free pathogen nucleic acid molecules originate from. In some cases, nucleotide variants at a genomic site are excluded from the analysis if more than 1, 2, or 3 nucleotide variants are identified in a given biological sample.

In an illustrative example, targeted sequencing with capture enrichment is used to analyze the cell-free viral DNA molecules in the circulation of NPC subjects and non-NPC subjects with detectable plasma EBV DNA. Capture probes can be designed to cover the whole EBV genome. In other cases, only part of the EBV genome can be analyzed, and capture probes are designed to cover only part of the EBV genome. In the same analysis, capture probes can also be included to target genomic regions of interest in the human genome. For instance, probes that target human common single nucleotide polymorphism (SNP) sites and human leukocyte antigen (HLA) SNPs can be included. In one embodiment, more probes can be designed to hybridize to other viral genomic sequences, for instance, HPV or HBV genomes.

In some cases, the variant pattern of the pathogen genome is analyzed via direct comparison between the sequence reads mapped to the reference genome and the reference genome. The comparison result can be further processed in any appropriate manner, for instance, for clustering analysis or phylogenetic tree analysis. Available bioinformatic tools for these analysis can include MEGA4, MEGA5, CLUSTALW, Phylip, RAxML, BEAST, PhyML, TreeView, MAFFT, MrBayes, BIONJ, MLTreeMap, Newick Utilities, Phylo.io, Phylogeny.fr, REALPHY, SuperTree, and The PhylOgenetic Web Repeater (POWER). The cluster analysis or phylogenetic tree analysis compares the sequence reads mapped to the pathogen reference genome with one or more pathogen genomes that are obtained from diseased tissues or healthy subject, or indicated as being able or unable to cause the pathogen-associated disorder, or indicated as being effective or ineffective in causing the pathogen-associated disorder.

In an illustrative example, the methods and systems provided herein include a block-based variant pattern analysis. The block-based variant pattern analysis can include segregating the reference genome of the pathogen into a plurality of bins (“blocks”). The sequence reads mapped to the pathogen reference genome are compared against a disorder-associated pathogen genome within each of the plurality of the bins. In some cases, there are multiple, such as, at least 2, 3, 4, 5, 6, 7, 8, 9, 10, 12, 14, 16, 18, 20, 22, 24, 26, 28, 30, 40, 50, 60, 70, 80, 90, 100, 120, 140, 160, 180, 200, 300, 400, 500, 600, 700, 800, 900, or 1000 different pathogen genomes to be compared with for the block-based analysis, including disorder-associated pathogen genome, and optionally pathogen genomes that are known or indicated as being unable to or ineffective in causing the pathogen-associated disorder (disorder-irrelevant pathogen genome). In the block-based analysis, within each of the plurality of bins, a similarity index is calculated based on the shared nucleotide variants between the sequence reads mapped to the pathogen reference genome and each of the disorder-associated pathogen genomes or the disorder-irrelevant pathogen genomes. The similarity index can be dependent on the proportion of the variant sites at which at least one of the sequence reads mapped to the pathogen reference genome has a same nucleotide variant as the disorder-associated or disorder-irrelevant pathogen genome. Based on the similarity index against each of the pathogen genomes that the sequence reads are compared against, a bin score can be calculated based on, for instance, the similarity level as reflected by the similarity index. In one instance, the bin score can be dependent on the proportion of the similarity indices above a predetermined cutoff. There can be a cutoff set for the similarity index, for instance, about 0.6, 0.7, 0.75, 0.8, 0.85, 0.9, or 0.95. Similarity index above the cutoff can indicate the sequence reads are “similar” to the pathogen genome it's compared against. Based on the analysis described above, pattern analysis can then be performed on a larger scale across the pathogen genome or part of the pathogen genome using the calculated similarity indices or the bin scores. Clustering analysis or phylogenetic analysis similar to the ones described above can follow the block-based analysis for predicting the risk for the development of the pathogen-associated disorder, such as, EBV-associated NPC.

### Risk Score

Some aspects of the present disclosure relates to stratification of the risk for a subject to develop the pathogen-associated disorder base on combinatorial consideration of one or more characteristics of the cell-free nucleic acid molecules from the pathogen in a biological sample from the subject. In some cases, a risk score is generated indicating the risk for the subject to develop the pathogen-associated disorder, e.g., EBV-associated nasopharyngeal cancer.

In some cases, the present disclosure relates to stratification of the risk for a subject to develop the pathogen-associated disorder base on combinatorial consideration of one or more characteristics of the cell-free nucleic acid molecules from the pathogen in a biological sample from the subject, and one or more factors of age of the subject, smoking habit of the subject, family history of NPC of the subject, genotypic factors of the subject, dietary history, or ethnicity of the subject. There can be a positive correlation between the positive rate for detection of plasma EBV DNA in a subject that has no clinically detectable NPC and the age of the subject. Smoking habit of the subject can render higher risk for the subject to develop NPC. Subjects having family history of NPC can have higher risk developing NPC themselves. Genotypic factors such as HLA status, as demonstrated in Bei et al. Nat Genet. 2010; 42:599-603, and Hildesheim et al. J Natl Cancer Inst. 2002; 94:1780-9, each of which is incorporated herein in its entirety, can also be correlated with the risk for NPC. In addition, dietary history can be correlated with risk for NPC, for instance subject having high consumption of salted fish can have a relatively high risk for NPC. Certain ethnicity, such as Cantonese, can also be associated with high risk for developing NPC.

In some cases, the methods and systems further include generating a report indicative of the risk for the subject to develop a pathogen-associated disorder. Such a report can have a numeric risk score value or a categorical risk evaluation. In some cases, the report includes recommendation for screening frequency or a future time point for follow-up screening assay. The report can be provided to the subject, a healthcare institution or a healthcare professional that serves the subject, or any relevant third-party such as a medical insurance company. The report can be reviewed, assessed, or edited by a certified doctor before or after release of the report. In some cases, a certified doctor provides additional comments on the risk evaluation or contributes to the final risk evaluation based on his/her medical opinion or independent exams.

In some cases, the present disclosure provides methods of stratifying risk for developing a pathogen-associated disorder, such as pathogen-associated proliferative disorder, such as EBV-associated NPC, by using a classifier. Such a classifier can take one or more factors described herein as a data input and provide an output comprising a risk score, which can be indicative of the risk for the subject to develop the pathogen-associated disorder. The one or more factors that can be fed into the classifier can include one or more characteristics of cell-free pathogen nucleic acid molecules, one or more characteristics of the cell-free nucleic acid molecules from the pathogen in a biological sample from the subject, and one or more factors of age of the subject, smoking habit of the subject, family history of NPC of the subject, genotypic factors of the subject, dietary history, and ethnicity of the subject. The risk score as an output of the classifier can be indicative of the risk for the subject to currently suffer from or develop the pathogen-associated disorder in the future. In some cases, the risk score is indicative of a possibility for the subject to currently suffer from the pathogen-associate disorder. In some cases, the risk score is indicative of a possibility for the subject to develop the pathogen-associated disorder within a future time duration, such as, but not limited to, within 1 year, 2 years, 3 years, 4 years, 5 years, 10 years, or 15 years. In some cases, the classifier provides an output comprising a recommended screening frequency or a future time point for follow-up screening assay. Such an output can be in the form of clinical recommendation or provided in a report as discussed above to the subject, a healthcare institution or a healthcare professional, or any third-party such as a medical insurance company.

As described herein, a classifier can refer to any algorithm that implements classification. In the present disclosure, the classifier can be a classification model built upon any appropriate algorithm for predicting the risk for future development of the pathogen-associated disorder. Appropriate algorithms can include machine learning algorithms and other mathematics/statistics models, such as, but not limited to, support vector machine (SVM), Naïve Bayes, logistics regression, random forest, decision tree, gradient boosting tree, neural network, deep learning, linear/kernel SVM, linear/non-linear regressions, linear discriminative analysis etc. In some cases, the classifier is a trained with a labeled dataset that includes a plurality of input-output pairs. For instance, a dataset generated from analysis results of samples from a number of subjects that have been diagnosed as having no NPC or having NPC. In these instances, the dataset can include input having one or more factors of characteristics of plasma EBV DNA from these subjects (e.g., variant pattern, methylation status, detectability/copy number, or fragment size), age, family history, smoking habits, ethnicity, or dietary history, as well as a corresponding output that indicates whether or not the corresponding subject has or has not NPC. In an illustrative example, the classifier can be trained with a labeled dataset that includes a large number of input-output pairs, such as at least 10, 20, 50, 100, 200, 500, 1000, 2000, 5000, 10000, or 20000 pairs.

In one example, a classification model is provided to predict the risk of future NPC development for subjects with detectable plasma EBV DNA using the analysis of the variant patterns. The classification model can be a classifier constructed as follows using a support vector machine (SVM) algorithm:

Given a training dataset comprising n samples:

(M1,Y1), . . . ,(Mn,Yn)
where Yi indicates the NPC status of sample i. Yi is 1 for a sample from a NPC patient) or −1 for a sample from a subject without NPC; Mi is a p-dimensional vector comprising the viral variant patterns for a sample i. For example, Mi can be a series of variant sites (e.g., 29 variant sites associated with NPC or 661 variant sites associated with NPC as set forth in Table 6). Alternatively, Mi can be a series of block-based variant similarity scores (e.g., a non-overlapping windows of 500 bp) with respect to the reference EBV variants present in subjects known to have NPC.

A “hyperplane” can be identified that separates the non-NPC and NPC groups as accurate as possible in a training dataset, by looking for a set of coefficients (W with p-dimensional vector) satisfying:

W·Mᵢ−b≥1 (for any subject in the NPC group) Criterion 1:

and

W·Mᵢ−b≤−1 (for any Subject in the Non-NPC Group) Criterion 2
where W is a p-dimensional vector of coefficients determining the hyperplane; M is a matrix (p×n dimensions) with p variants (or block-based similarity scores) and n samples; b is the intercept.

The two criteria (i.e. criteria 1 and 2) can also be written as:

Yi(W*Mi−b)≥1 (criterion 3)

where Yi is either −1 (non-NPC) or 1 (NPC).

The margin distance (D) between criteria 1 and 2 is:

$$\frac{2}{‖ W ‖} ,$$

where ∥W∥ is computed using the distance from a point to a plane equation.

D is to be maximized by minimizing ∥W∥ subject to criterion 3.

Based on this principle, the parameters (W and b) of the classifier can be determined. The trained classifier, implemented with the trained parameters (W and b), can thus be used to calculate NPC risk score for test samples.

In one illustrative example, NPC risk score is calculated as the weighted summation of EBV genotypes at a fixed set of SNV sites across the viral genome (as explanatory variables in a binary logistic regression model). In the example, a set of NPC-associated SNVs is identified by analyzing the difference in the EBV SNV profiles from NPC and non-NPC samples in the training set. The association of each variant across the EBV genome with the NPC cases can be analyzed, e.g., using Fisher's exact test. Then a fixed set of significant SNVs can be obtained, e.g., with a false discovery rate (FDR) controlled at 5%. The NPC risk score of a test sample can be determined by its EBV genotypes over this specific set of significant SNV sites identified from a training set that comprises sequencing data from plasma DNA samples from known NPC and non-NPC subjects. In some cases, plasma EBV DNA molecules can have a low concentration, thus there can be incomplete coverage of the whole EBV genome by the sequenced EBV DNA reads. The score can be formulated to be determined by the genotypic patterns over those SNV sites which are covered by plasma EBV DNA reads (e.g., with available genotypic information). To derive the NPC risk score, the subset of significant SNV sites covered by plasma EBV DNA reads in a sample can be identified first, and then the weighting (effect sizes) of genotypes at each site can be determined within the subset of significant SNV sites. A logistic regression model as follows can be constructed to inform the effect sizes of the risk genotypes at each SNV site on NPC:

$$P = \frac{1}{1 + {e}^{- ( {β}_{0} + {\sum }_{k = 1}^{n} {β}_{k} {X}_{k} )}}$$

which can be rewritten as:

$$logit ( P ) = log ( \frac{P}{1 - P} ) = {β}_{0} + {Σ}_{k = 1}^{n} {β}_{k} {X}_{k} ,$$

where n is the number of significant SNV sites; β₀ and βₖ are the coefficients which could be determined by maximum likelihood estimator; P is the probability of the EBV-positive patient having NPC; the variable Xₖ represents the SNV site at genomic position k. Xₖ can be coded as −1, if a variant present in a sample identical to the EBV reference genome. Xₖ can be coded as 1, if an alternative variant present in a sample. Xₖ can be coded as 0, if the analyzed variant site is not covered in a sample. The coefficients β₀ and βₖ can thus be estimated, e.g., using ‘LogisticRegression’ function in python. This can be achieved by analyzing the genotypic patterns at each site among the NPC and non-NPC samples in the training dataset. NPC risk score of a test sample can thus be derived based on its own genotypes at SNV sites, weighted by the corresponding coefficients β₀ and βₖ deduced from the training model.

### Biological Sample

The biological sample used in methods as provided herein can include any tissue or material derived from a living or dead subject. A biological sample can be a cell-free sample. A biological sample can include a nucleic acid (e.g., DNA or RNA) or a fragment thereof. The nucleic acid in the sample can be a cell-free nucleic acid. A sample can be a liquid sample or a solid sample (e.g., a cell or tissue sample). The biological sample can be a bodily fluid, such as blood, plasma, serum, urine, oral rinse fluid, nasal flushing fluid, nasal brush sample, vaginal fluid, fluid from a hydrocele (e.g., of the testis), vaginal flushing fluids, pleural fluid, ascitic fluid, cerebrospinal fluid, saliva, sweat, tears, sputum, bronchoalveolar lavage fluid, discharge fluid from the nipple, aspiration fluid from different parts of the body (e.g., thyroid, breast), etc. Stool samples can also be used. In various examples, the majority of DNA in a biological sample that has been enriched for cell-free DNA (e.g., a plasma sample obtained via a centrifugation protocol) can be cell-free (e.g., greater than 50%, 60%, 70%, 80%, 90%, 95%, or 99% of the DNA can be cell-free). The biological sample can be treated to physically disrupt tissue or cell structure (e.g., centrifugation and/or cell lysis), thus releasing intracellular components into a solution which can further contain enzymes, buffers, salts, detergents, and the like which are used to prepare the sample for analysis.

Methods and systems provided herein can be used to analyze nucleic acid molecules in a biological sample. The nucleic acid molecules can be cellular nucleic acid molecules, cell-free nucleic acid molecules, or both. The cell-free nucleic acids used by methods as provided herein can be nucleic acid molecules outside of cells in a biological sample. The cell-free nucleic acid molecules can be present in various bodily fluids, e.g., blood, saliva, semen, and urine. Cell-free DNA molecules can be generated owing to cell death in various tissues that can be caused by health conditions and/or diseases, e.g., viral infection and tumor growth. Cell-free nucleic acid molecules can include sequences generated as a result of pathogen integration events.

Cell-free nucleic acid molecules, e.g., cell-free DNA, used in methods as provided herein can exist in plasma, urine, saliva, or serum. Cell-free DNA can occur naturally in the form of short fragments. Cell-free DNA fragmentation can refer to the process whereby high molecular weight DNA (such as DNA in the nucleus of a cell) are cleaved, broken, or digested to short fragments when cell-free DNA molecules are generated or released. Methods and systems provided herein can be used to analyze cellular nucleic acid molecules in some cases, for instance, cellular DNA from a tumor tissue, or cellular DNA from white blood cells when the patient has leukemia, lymphoma, or myeloma. Sample taken from a tumor tissue can be subject to assays and analyses according to some examples of the present disclosure.

### Subjects

Methods and systems provided herein can be used to analyze sample from a subject, e.g., organism, e.g., host organism. The subject can be any human patient, such as a cancer patient, a patient at risk for cancer, or a patient with a family or personal history of cancer. In some cases, the subject is in a particular stage of cancer treatment. In some cases, the subject can have or be suspected of having cancer. In some cases, whether the subject has cancer is unknown.

In some cases, depending on the result of the screening assay provided herein, the subject receives or does not receive a medical treatment of the pathogen-associated disorder. In one example, while the first screening assay shows positive results, indicating a high risk for the subject to develop a pathogen-associated disorder, the subject is diagnosed as not having the pathogen-associated disorder (e.g., EBV-associated NPC) by a follow-on diagnostic examination. In this case, the subject does not receive a medical treatment, such as, but not limited to, treatment with therapeutic agents (e.g., chemotherapy), radiotherapy, surgery, or any combination thereof. In another example, the subject is screened as having a high risk for developing a pathogen-associated disorder (e.g., HPV-associated cervical cancer) and further diagnosed as having the disorder. As a result, the subject can receive a medical treatment of the disorder, such as, but not limited to, surgery, chemotherapy, radiotherapy, targeted therapy, immunotherapy, or any combination thereof.

Pathogen-associated disorders that the methods and systems provided herein can be applicable to can include proliferative disorders, e.g., cancers. The disorders can be associated with or caused by pathogens such as viruses, bacterium, or fungi. The viruses that can be associated with the disorders described herein can include EBV, Kaposi's sarcoma-associated herpesvirus (KSHV), HPV (for example but not limited to HPV 16, 18, 31, 33, 34, 35, 39, 45, 51, 52, 56, 58, 59, 66, 68 and 70) (Burd et al. Clin Microbiol Rev 2003:16:1-17), Merkel cell polyomavirus (MCPV), HBV, HCV and Human T-lymphotrophic virus-1 (HTLV1). Applicable pathogen-associated cancers can include Burkitt's lymphoma, Hodgkin's lymphoma, immunosuppression-related lymphoma, T and NK cell lymphomas; nasopharyngeal, or stomach carcinomas, which can be associated with EBV. Applicable pathogen-associated cancers can include primary effusion lymphoma or Kaposi sarcoma, which can be associated with KSHV. Applicable pathogen-associated cancers can include cervical, head and neck cancers, or anogenital tract carcinomas, which can be associated with HPV. Applicable pathogen-associated cancers can include Merkel cell carcinoma that is associated with MCPV. Applicable pathogen-associated cancers can include HCC that can be associated with HBV or hepatitis C virus (HCV). Applicable pathogen-associated cancers can include Adult T-cell leukemia/lymphoma that can be associated with HTLV1.

A subject can have any type of cancer or tumor or have risk for developing any type of cancer or tumor. In an example, a subject can have nasopharyngeal cancer, or cancer of the nasal cavity. In another example, a subject can have oropharyngeal cancer, or cancer of the oral cavity. Non-limiting examples of cancer can include, but are not limited to, adrenal cancer, anal cancer, basal cell carcinoma, bile duct cancer, bladder cancer, cancer of the blood, bone cancer, a brain tumor, breast cancer, bronchus cancer, cancer of the cardiovascular system, cervical cancer, colon cancer, colorectal cancer, cancer of the digestive system, cancer of the endocrine system, endometrial cancer, esophageal cancer, eye cancer, gallbladder cancer, a gastrointestinal tumor, hepatocellular carcinoma, kidney cancer, hematopoietic malignancy, laryngeal cancer, leukemia, liver cancer, lung cancer, lymphoma, melanoma, mesothelioma, cancer of the muscular system, Myelodysplastic Syndrome (MDS), myeloma, nasal cavity cancer, nasopharyngeal cancer, cancer of the nervous system, cancer of the lymphatic system, oral cancer, oropharyngeal cancer, osteosarcoma, ovarian cancer, pancreatic cancer, penile cancer, pituitary tumors, prostate cancer, rectal cancer, renal pelvis cancer, cancer of the reproductive system, cancer of the respiratory system, sarcoma, salivary gland cancer, skeletal system cancer, skin cancer, small intestine cancer, stomach cancer, testicular cancer, throat cancer, thymus cancer, thyroid cancer, a tumor, cancer of the urinary system, uterine cancer, vaginal cancer, or vulvar cancer. The lymphoma can be any type of lymphoma including B-cell lymphoma (e.g., diffuse large B-cell lymphoma, follicular lymphoma, small lymphocytic lymphoma, mantle cell lymphoma, marginal zone B-cell lymphoma, Burkitt lymphoma, lymphoplasmacytic lymphoma, hairy cell leukemia, or primary central nervous system lymphoma) or a T-cell lymphoma (e.g., precursor T-lymphoblastic lymphoma, or peripheral T-cell lymphoma). The leukemia can be any type of leukemia including acute leukemia or chronic leukemia. Types of leukemia include acute myeloid leukemia, chronic myeloid leukemia, acute lymphocytic leukemia, acute undifferentiated leukemia, or chronic lymphocytic leukemia. In some cases, the cancer patient does not have a particular type of cancer. For example, in some instances, the patient can have a cancer that is not breast cancer.

Examples of cancer include cancers that cause solid tumors as well as cancers that do not cause solid tumors. Furthermore, any of the cancers mentioned herein can be a primary cancer (e.g., a cancer that is named after the part of the body where it first started to grow) or a secondary or metastatic cancer (e.g., a cancer that has originated from another part of the body).

A subject diagnosed by any of the methods described herein can be of any age and can be an adult, infant or child. In some cases, the subject is 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 years old, or within a range therein (e.g., between 2 and 20 years old, between 20 and 40 years old, or between 40 and 90 years old). A particular class of patients that can benefit can be patients over the age of 40. Another particular class of patients that can benefit can be pediatric patients. Furthermore, a subject diagnosed by any of the methods or compositions described herein can be male or female.

In some embodiments, a method of the present disclosure can detect a tumor or cancer in a subject, wherein the tumor or cancer has a geographic pattern of disease. In an example, a subject can have an EBV-related cancer (e.g., nasopharyngeal cancer), which is prevalent in South China (e.g., Hong Kong SAR). In another example, subject can have an HPV-related cancer (e.g., oropharyngeal cancer), which can be prevalent in the United States and Western Europe. In yet another example, a subject can have a HTLV-1-related cancer (e.g., adult T-cell leukemia/lymphoma), which can be prevalent in southern Japan, the Caribbean, central Africa, parts of South America, and in some immigrant groups in the southeastern United States.

Any of the methods disclosed herein can also be performed on a non-human subject, such as a laboratory or farm animal, or a cellular sample derived from an organism disclosed herein. Non-limiting examples of a non-human subject include a dog, a goat, a guinea pig, a hamster, a mouse, a pig, a non-human primate (e.g., a gorilla, an ape, an orangutan, a lemur, or a baboon), a rat, a sheep, a cow, or a zebrafish.

### Computer System

Any of the methods disclosed herein can be performed and/or controlled by one or more computer systems. In some examples, any step of the methods disclosed herein can be wholly, individually, or sequentially performed and/or controlled by one or more computer systems. Any of the computer systems mentioned herein can utilize any suitable number of subsystems. In some embodiments, a computer system includes a single computer apparatus, where the subsystems can be the components of the computer apparatus. In other embodiments, a computer system can include multiple computer apparatuses, each being a subsystem, with internal components. A computer system can include desktop and laptop computers, tablets, mobile phones and other mobile devices.

The subsystems can be interconnected via a system bus. Additional subsystems include a printer, keyboard, storage device(s), and monitor that is coupled to display adapter. Peripherals and input/output (I/O) devices, which couple to I/O controller, can be connected to the computer system by any number of connections known in the art such as an input/output (I/O) port (e.g., USB, FireWire®). For example, an I/O port or external interface (e.g., Ethernet, Wi-Fi, etc.) can be used to connect computer system to a wide area network such as the Internet, a mouse input device, or a scanner. The interconnection via system bus allows the central processor to communicate with each subsystem and to control the execution of a plurality of instructions from system memory or the storage device(s) (e.g., a fixed disk, such as a hard drive, or optical disk), as well as the exchange of information between subsystems. The system memory and/or the storage device(s) can embody a computer readable medium. Another subsystem is a data collection device, such as a camera, microphone, accelerometer, and the like. Any of the data mentioned herein can be output from one component to another component and can be output to the user.

A computer system can include a plurality of the same components or subsystems, e.g., connected together by external interface or by an internal interface. In some embodiments, computer systems, subsystem, or apparatuses can communicate over a network. In such instances, one computer can be considered a client and another computer a server, where each can be part of a same computer system. A client and a server can each include multiple systems, subsystems, or components.

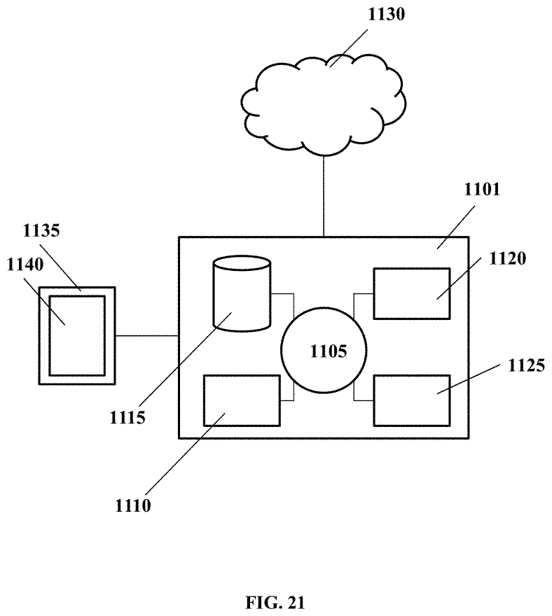

The present disclosure provides computer control systems that are programmed to implement methods of the disclosure for stratifying a risk for pathogen-associated disorder. FIG. 21 shows a computer system 1101 that is programmed or otherwise configured to analyze cell-free nucleic acid molecules or sequence reads thereof, analyze other factors associated with the risk for the disorder, evaluate the risk, or generate a report indicative of the risk as described herein. The computer system 1101 can implement and/or regulate various aspects of the methods provided in the present disclosure, such as, for example, controlling sequencing of the nucleic acid molecules from a biological sample, performing various steps of the bioinformatics analyses of sequencing data as described herein, integrating data collection, analysis and result reporting, and data management. The computer system 1101 can be an electronic device of a user or a computer system that is remotely located with respect to the electronic device. The electronic device can be a mobile electronic device.

The computer system 1101 includes a central processing unit (CPU, also “processor” and “computer processor” herein) 1105, which can be a single core or multi core processor, or a plurality of processors for parallel processing. The computer system 1101 also includes memory or memory location 1110 (e.g., random-access memory, read-only memory, flash memory), electronic storage unit 1115 (e.g., hard disk), communication interface 1120 (e.g., network adapter) for communicating with one or more other systems, and peripheral devices 1125, such as cache, other memory, data storage and/or electronic display adapters. The memory 1110, storage unit 1115, interface 1120 and peripheral devices 1125 are in communication with the CPU 1105 through a communication bus (solid lines), such as a motherboard. The storage unit 1115 can be a data storage unit (or data repository) for storing data. The computer system 1101 can be operatively coupled to a computer network (“network”) 1130 with the aid of the communication interface 1120. The network 1130 can be the Internet, an internet and/or extranet, or an intranet and/or extranet that is in communication with the Internet. The network 1130 in some cases is a telecommunication and/or data network. The network 1130 can include one or more computer servers, which can enable distributed computing, such as cloud computing. The network 1130, in some cases with the aid of the computer system 1101, can implement a peer-to-peer network, which may enable devices coupled to the computer system 1101 to behave as a client or a server.

The CPU 1105 can execute a sequence of machine-readable instructions, which can be embodied in a program or software. The instructions may be stored in a memory location, such as the memory 1110. The instructions can be directed to the CPU 1105, which can subsequently program or otherwise configure the CPU 1105 to implement methods of the present disclosure. Examples of operations performed by the CPU 1105 can include fetch, decode, execute, and writeback.

The CPU 1105 can be part of a circuit, such as an integrated circuit. One or more other components of the system 1101 can be included in the circuit. In some cases, the circuit is an application specific integrated circuit (ASIC).

The storage unit 1115 can store files, such as drivers, libraries and saved programs. The storage unit 1115 can store user data, e.g., user preferences and user programs. The computer system 1101 in some cases can include one or more additional data storage units that are external to the computer system 1101, such as located on a remote server that is in communication with the computer system 1101 through an intranet or the Internet.

The computer system 1101 can communicate with one or more remote computer systems through the network 1130. For instance, the computer system 1101 can communicate with a remote computer system of a user (e.g., a Smart phone installed with application that receives and displays results of sample analysis sent from the computer system 1101). Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 1101 via the network 1130.

Methods as described herein can be implemented by way of machine (e.g., computer processor) executable code stored on an electronic storage location of the computer system 1101, such as, for example, on the memory 1110 or electronic storage unit 1115. The machine executable or machine readable code can be provided in the form of software. During use, the code can be executed by the processor 1105. In some cases, the code can be retrieved from the storage unit 1115 and stored on the memory 1110 for ready access by the processor 1105. In some situations, the electronic storage unit 1115 can be precluded, and machine-executable instructions are stored on memory 1110.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 1101, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that include a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 1101 can include or be in communication with an electronic display 1135 that includes a user interface (UI) 1140 for providing, for example, results of sample analysis, such as, but not limited to graphic showings of pathogen integration profile, genomic location of pathogen integration breakpoints, classification of pathology (e.g., type of disease or cancer and level of cancer), and treatment suggestion or recommendation of preventive steps based on the classification of pathology. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 1105. The algorithm can, for example, control sequencing of the nucleic acid molecules from a sample, direct collection of sequencing data, analyzing the sequencing data, performing block-based variant pattern analysis, evaluating the risk, or generating the report indicative of the risk.

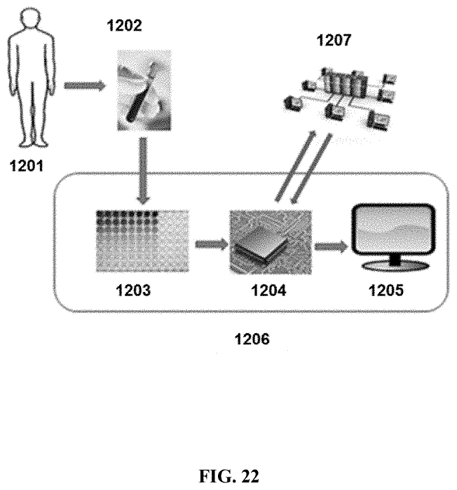

In some cases, as shown in FIG. 22, a sample 1202 may be obtained from a subject 1201, such as a human subject. A sample 1202 may be subjected to one or more methods as described herein, such as performing an assay. In some cases, an assay may include hybridization, amplification, sequencing, labeling, epigenetically modifying a base, or any combination thereof. One or more results from a method may be input into a processor 1204. One or more input parameters such as a sample identification, subject identification, sample type, a reference, or other information may be input into a processor 1204. One or more metrics from an assay may be input into a processor 1204 such that the processor may produce a result, such as a classification of pathology (e.g., diagnosis) or a recommendation for a treatment. A processor may send a result, an input parameter, a metric, a reference, or any combination thereof to a display 1205, such as a visual display or graphical user interface. A processor 1204 may (i) send a result, an input parameter, a metric, or any combination thereof to a server 1207, (ii) receive a result, an input parameter, a metric, or any combination thereof from a server 1207, (iii) or a combination thereof.

Aspects of the present disclosure can be implemented in the form of control logic using hardware (e.g., an application specific integrated circuit or field programmable gate array) and/or using computer software with a generally programmable processor in a modular or integrated manner. As used herein, a processor includes a single-core processor, multi-core processor on a same integrated chip, or multiple processing units on a single circuit board or networked. Based on the disclosure and teachings provided herein, a person of ordinary skill in the art will know and appreciate other ways and/or methods to implement embodiments described herein using hardware and a combination of hardware and software.

Any of the software components or functions described in this application can be implemented as software code to be executed by a processor using any suitable computer language such as, for example, Java, C, C++, C #, Objective-C, Swift, or scripting language such as Perl or Python using, for example, conventional or object-oriented techniques. The software code can be stored as a series of instructions or commands on a computer readable medium for storage and/or transmission. A suitable non-transitory computer readable medium can include random access memory (RAM), a read only memory (ROM), a magnetic medium such as a hard-drive or a floppy disk, or an optical medium such as a compact disk (CD) or DVD (digital versatile disk), flash memory, and the like. The computer readable medium can be any combination of such storage or transmission devices.

Such programs can also be encoded and transmitted using carrier signals adapted for transmission via wired, optical, and/or wireless networks conforming to a variety of protocols, including the Internet. As such, a computer readable medium can be created using a data signal encoded with such programs. Computer readable media encoded with the program code can be packaged with a compatible device or provided separately from other devices (e.g., via Internet download). Any such computer readable medium can reside on or within a single computer product (e.g., a hard drive, a CD, or an entire computer system), and can be present on or within different computer products within a system or network. A computer system can include a monitor, printer, or other suitable display for providing any of the results mentioned herein to a user.

Any of the methods described herein can be totally or partially performed with a computer system including one or more processors, which can be configured to perform the steps. Thus, embodiments can be directed to computer systems configured to perform the steps of any of the methods described herein, with different components performing a respective steps or a respective group of steps. Although presented as numbered steps, steps of methods herein can be performed at a same time or in a different order. Additionally, portions of these steps can be used with portions of other steps from other methods. Also, all or portions of a step can be optional. Additionally, any of the steps of any of the methods can be performed with modules, units, circuits, or other approaches for performing these steps.

### Other Embodiments

The section headings used herein are for organizational purposes only and are not to be construed as limiting the subject matter described.

It is to be understood that the methods described herein are not limited to the particular methodology, protocols, subjects, and sequencing techniques described herein and as such can vary. It is also to be understood that the terminology used herein is for the purpose of describing particular embodiments only, and is not intended to limit the scope of the methods and compositions described herein, which will be limited only by the appended claims. While some embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein can be employed in practicing the disclosure. It is intended that the following claims define the scope of the disclosure and that methods and structures within the scope of these claims and their equivalents be covered thereby.

Several aspects are described with reference to example applications for illustration. Unless otherwise indicated, any embodiment can be combined with any other embodiment. It should be understood that numerous specific details, relationships, and methods are set forth to provide a full understanding of the features described herein. A skilled artisan, however, will readily recognize that the features described herein can be practiced without one or more of the specific details or with other methods. The features described herein are not limited by the illustrated ordering of acts or events, as some acts can occur in different orders and/or concurrently with other acts or events. Furthermore, not all illustrated acts or events are required to implement a methodology in accordance with the features described herein.

### EXAMPLES

The following examples are provided to further illustrate some embodiments of the present disclosure, but are not intended to limit the scope of the disclosure; it will be understood by their exemplary nature that other procedures, methodologies, or techniques known to those skilled in the art may alternatively be used.

### Example 1. NPC Screening on a Cohort of Over 20,000 Subjects Over 4 Years

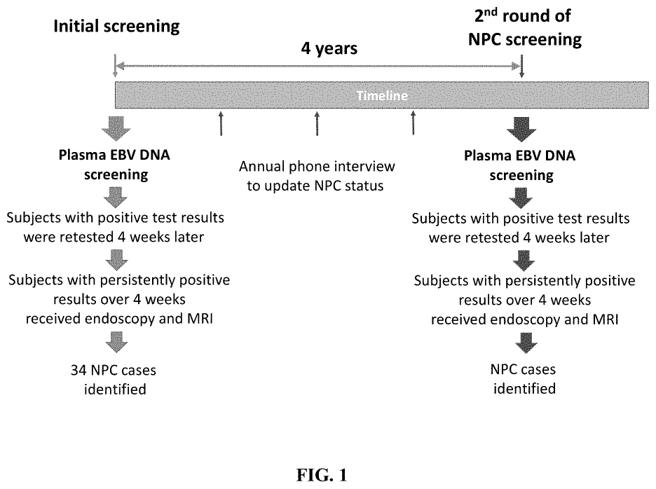

This example describes a large-scale screening study performed on a cohort of over 20,000 subjects over about 4 years. FIG. 1 shows a diagram of the design of this study. In the initial round of screening, over 20,000 men, with ages between 40 to 62 years, were screened for NPC using plasma EBV DNA analysis. Subjects with detectable plasma EBV DNA were retested after a median of 4 weeks with a second set of blood samples. This arrangement was aimed to differentiate NPC patients from those without NPC but with detectable plasma EBV DNA. In a previous study, it was shown that the presence of plasma EBV DNA in subjects without NPC was typically a transient phenomenon. In two-thirds of these individuals, the plasma EBV DNA would become undetectable at a median of two weeks later. Subjects with persistently positive plasma EBV DNA results were further investigated with nasal endoscopy and magnetic resonance imaging (MRI) of the nasopharynx to confirm or rule out the presence of NPC. Based on this arrangement, 34 cases of NPC were identified.

Later, another round (second round) of NPC screening on the cohort was performed at a median of 4 years after the initial round of screening. In the second round of NPC screening, again subjects with positive test results would be retested approximately 4 weeks later as in the first round of screening. Subjects with positive results on two consecutive testing over 4 weeks would be further investigated with nasal endoscopy and MRI. The second round of screening was started in 2017. A total of 8335 subjects had completed the second round of screening up to 15 Sep. 2018. 784 (9.4%) subjects were positive for plasma EBV DNA. On the retesting at four weeks, 230 (2.7%) subjects still had detectable plasma EBV DNA. Table 1 summarizes the test results in both rounds of NPC screening.

TABLE 1
Status of Plasma EBV DNA in the first and second rounds of
NPC screening

| Plasma EBV | | | | |
|---|---|---|---|---|
| DNA status in | Plasma EBV DNA status in the second-round screening | | | |
| the first-round | | | Transiently | Persistently |
| screening | Number | Negative | positive | positive |
| Negative | 7907 | 7267 (92%) | 479 (6%) | 161 (2%) |
| Transiently | 276 | 218 (79%) | 30 (11%) | 28 (10%) |
| positive | | | | |
| Persistently | 152 | 66 (43%) | 48 (32%) | 38 (25%) |
| positive | | | | |

As shown in Table 1, the probability of having detectable plasma EBV DNA in the second-round NPC screening was correlated with the status of plasma EBV DNA in the first-round of screening. Subjects with negative, transiently positive and persistently positive plasma EBV DNA in the first round of screening had 8%, 21% and 57% probabilities of having detectable plasma EBV DNA in the initial analysis of the second round of screening. Moreover, the chance of having persistently positive plasma EBV DNA at 4 weeks later was progressively increased across the three groups from 2% to 25%.

The NPC patients identified by the screening described herein had much earlier stage distribution than those in a historical cohort who did not receive NPC screening. The percentage of early-staged disease (Stages I and II) were 70% and 20%, respectively. This change in stage distribution resulted in a significant improvement in progression-free survival of patients with a hazard ratio of 0.1. Summarized in Table 2 are the stage distributions of NPC cases in both first and second rounds of screening. After screening of 8335 subjects in the second round, 13 new cases of NPC have been identified. The percentages of patients having early-staged disease were 71% and 69%, respectively, for the first and second round screenings. There was no significant difference in the percentage of patients with early-stage disease (P=0.93, chi-square test).

TABLE 2
Stage distributions of NPC cases identified in the two rounds
of screening

| | 1st round | 2nd round |
|---|---|---|
| Stage | screening | screening |
| I | 16 (47%) | 4 (31%) |
| II | 8 (24%) | 5 (38%) |
| III | 8 (24%) | 4 (31%) |
| IV | 2 (6%) | 0 (0%) |

As summarized in Table 3, subjects with transiently and persistently detectable plasma EBV DNA in the first round of screening had higher risk of having NPC detected in the second round of screening which were carried out 4 years after the first round, compared with those with undetectable plasma EBV DNA in the first round. The relative risk values are 7.2 and 19.7, respectively, for these two groups.

TABLE 3
Number of NPC cases identified in the second round screening
categorized by plasma EBV DNA status in the first round

| | | Number of NPC | Relative risk |
|---|---|---|---|
| | | detected in the | for NPC relative |
| Plasma EBV | | second round | to subjects with |
| DNA status in | | (% of subjects with | undetectable plasma |
| the first-round | | the same plasma | EBV DNA in |
| screening | Number | EBV DNA status) | the first round |
| Negative | 7907 | 8 (0.10%) | 1 |
| Transiently | 276 | 2 (0.72%) | 7.2 |
| positive | | | |
| Persistently | 152 | 3 (1.97%) | 19.7 |
| positive | | | |

These results suggest that plasma EBV DNA analysis is useful not only for the screening of the current status of having NPC, but also for predicting the risk of having clinically observable NPC in the future. One practical application of this finding can be for tailor-making the interval for repeating the screening based on the plasma EBV DNA status of a screened subject in an earlier instance. For example, subjects with detectable plasma EBV DNA at baseline but without identifiable NPC can be rescreened after a shorter interval compared with those with undetectable plasma EBV DNA. Also as illustration, the interval for repeating the screening can be 4 years, 2 years and 1 year for subjects with undetectable, transiently detectable and persistently detectable plasma EBV DNA, respectively.

### Example 2. NPC Screening Based on Detectability of Plasma EBV DNA

This example describes a NPC screening regimen designed for a subject based on the detectability of EBV DNA in the plasma of the subject. FIG. 2 shows a schematic of the regimen as described herein.

According to the regimen, a subject with undetectable plasma EBV DNA in an earlier instance of screening is rescreened 4 years later because the risk of NPC for subjects with undetectable EBV DNA in the coming 4 years would be relatively low. If the subsequent screening is negative for plasma EBV DNA, the interval for the subsequent screening is 4 years. However, when the subject has detectable EBV DNA on one screening occasion but with no NPC detected, the next screening is arranged one year later. The interval for screening is reverted back to 4 years when the plasma EBV DNA remains negative for 4 years. The actual time intervals used for specific screening programs is also adjusted according to health economic considerations (e.g. the cost of the screening), subject preference (e.g. a more frequent screening interval may be more disruptive for the lifestyles of certain subjects) and other clinical parameters (e.g. genotypes of the individual, family history of NPC, dietary history, ethnic origin (e.g. Cantonese)).

### Example 3. Variant Pattern Analysis of Cell-Free EBV DNA Molecules

In this example, targeted sequencing with capture enrichment was used to analyze the cell-free viral DNA molecules in the circulation of NPC subjects, non-NPC subjects with detectable plasma EBV DNA, and pre-NPC subjects (detailed in the subsequent section). Capture probes were designed to cover the whole EBV genome. In the same analysis, probes which target ˜3000 human common single nucleotide polymorphism (SNP) sites and human leukocyte antigen (HLA) SNPs were also included.

In this example, the plasma EBV DNA of 13 NPC patients and 16 non-NPC subjects with detectable plasma EBV DNA were analyzed. The 13 NPC patients presented symptomatically and were recruited from either the Department of Clinical Oncology or Department of Otorhinolaryngology of the Prince of Wales Hospital. The 16 non-NPC subjects were from the over 20,000-subject NPC screening cohort as described in Example 1.

In this analysis, targeted sequencing with capture enrichment by specifically designed capture probes was used. For each plasma sample analyzed, DNA was extracted from 4 mL plasma using the QlAamp Circulating Nucleic Acid Kit. For each case, all extracted DNA was used for the preparation of sequencing library using the TruSeq Nano DNA library preparation kit (Illumina). Barcoding was performed using a dual-indexing system incorporated with unique molecular identifier (UMI) sequences (xGen Dual Index UMI Adapters, Integrated DNA Technologies). Eight cycles of PCR amplification were performed on the adapter-ligated samples using the TruSeq Nano Kit (Illumina). The amplification products were then captured with the myBait custom capture panel system (Arbor Biosciences) using the custom-designed probes covering the viral and human genomic regions stated above. After the target capture, the captured products were enriched by 14 cycles of PCR to generate DNA libraries. The DNA libraries were sequenced on a NextSeq platform (Illumina). For each sequencing run, ten samples with unique sample barcodes were sequenced using the paired-end mode. Each DNA fragments would be sequenced 71 nucleotides from each of the two ends. After sequencing, the sequence reads would be mapped to an artificially combined reference sequence which consists of the whole human genome (hg19), the whole EBV genome (GenBank: AJ507799.2), the whole HBV genome and the whole HPV genome. The alignment was conducted with the use of SOAP2 (Bioinformatics 2009; 25:1966-7), allowing up to 2 mismatches for each read in a correct orientation with an insert size of no more than 600 bp. Sequenced reads mapping to unique positions in the combined genomic sequence would be used for downstream analysis. All duplicated fragments with the identical unique molecular identifier would be filtered.

Based on the alignment results, the nucleotide differences, including but not limited to single nucleotide variants (SNVs), between sequenced reads and the EBV reference genome (GenBank: AJ507799.2) were identified. Among the 44 samples from the 13 NPC subjects, 16 non-NPC subjects with detectable plasma EBV DNA and 4 pre-NPC subjects, a median of 1116 SNVs (interquartile range (IQR): 902-1216) were identified. In these plasma samples, two different alleles were observed at some nucleotide positions of the EBV genome. This observation can be due to sequencing errors or the presence of tumor heterogeneity. A median of only 26 positions (IQR: 20-35) had more than one allele in the plasma EBV DNA.

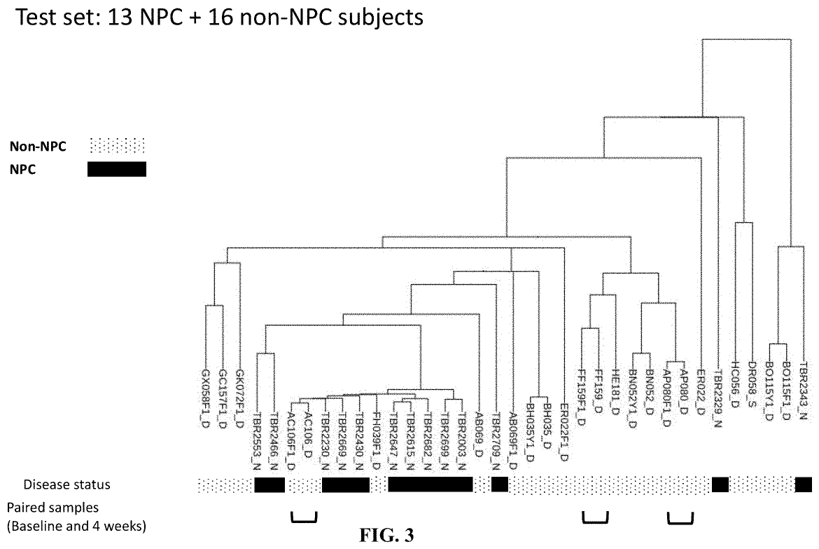

In the phylogenetic tree analysis as shown in FIG. 3, the NPC subjects were clustered together and were separated from the non-NPC subjects. These results suggested that there were different EBV variant profiles between NPC and non-NPC subjects. Hence, the EBV variant profile analysis of plasma EBV DNA could be used to differentiate NPC and non-NPC subjects in the context of screening. Three non-NPC subjects (AC106, AP080 and FF159) had two serially collected samples analyzed which were collected at 4 weeks apart. Two samples from the same individuals were clustered together indicating that they share very similar variants.

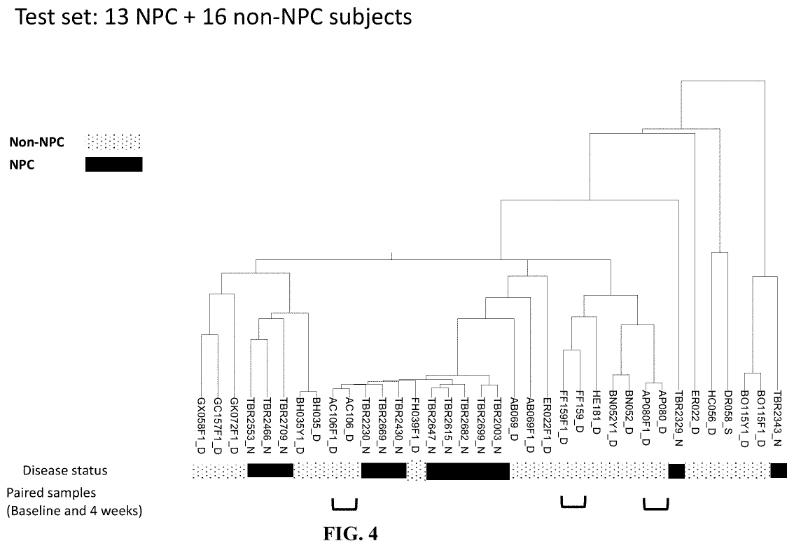

The phylogenetic tree analysis was also performed based on the EBV variants but excluding the 29 variants reported in the study by Hui et al ((Hui et al. Int J Cancer 2019, doi.org/10.1002/ijc.32049) on the same group of 13 NPC patients and 16 non-NPC subjects with detectable plasma EBV DNA. As shown in FIG. 4, the NPC subjects were also clustered together and separated from the non-NPC subjects.

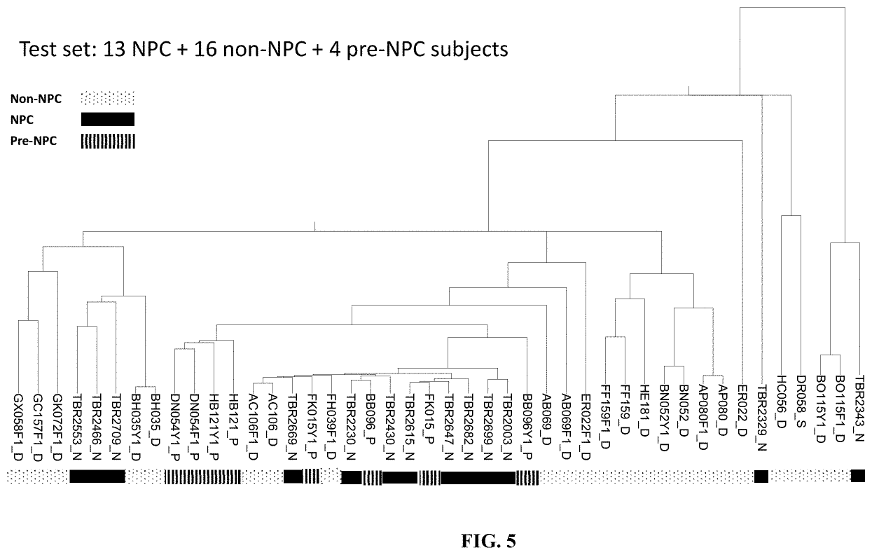

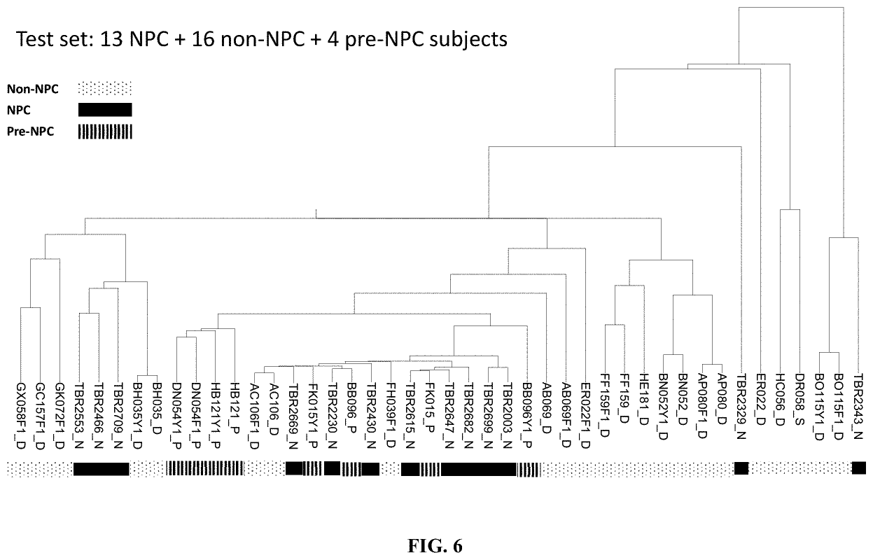

Four subjects who were persistently positive for plasma EBV DNA in the first round of screening (as described in Example 1) but with no detectable NPC on endoscopy and MRI, were subsequently diagnosed of having NPC. All of them (BB096, DN054, FK015 and HB121) were diagnosed of having NPC 3 years after the first round of screening. All of them had one additional plasma sample collected at 1 year after the first round of screening during their follow-up at the otolaryngology clinic. For each of these four subjects, two samples collected at first round of screening and 1 year later were analyzed for the EBV variants. As shown in FIG. 5, the samples from the pre-NPC subjects were clustered with the NPC samples, indicating that the EBV variants associated with NPC are present before the actual occurrence of the cancer. This suggests that those individuals with NPC-associated EBV variants are of higher risk of developing NPC in the future. The phylogenetic tree analysis was also performed based on the EBV variants but excluding the 29 variants reported in the study by Hui et al ((Hui et al. Int J Cancer 2019, doi.org/10.1002/ijc.32049) on the same group of NPC, non-NPC and pre-NPC subjects. As shown in FIG. 6, the samples from the pre-NPC subjects were still clustered with the NPC samples, further suggesting that that the analysis of the EBV variants would be able to predict the risk of NPC in the future.

### Example 4. Block-Based Variant Pattern Analysis

This example describes working principle of an exemplary block-based variant pattern analysis approach and its application to analysis of EBV variant pattern in samples as described in Example 3.

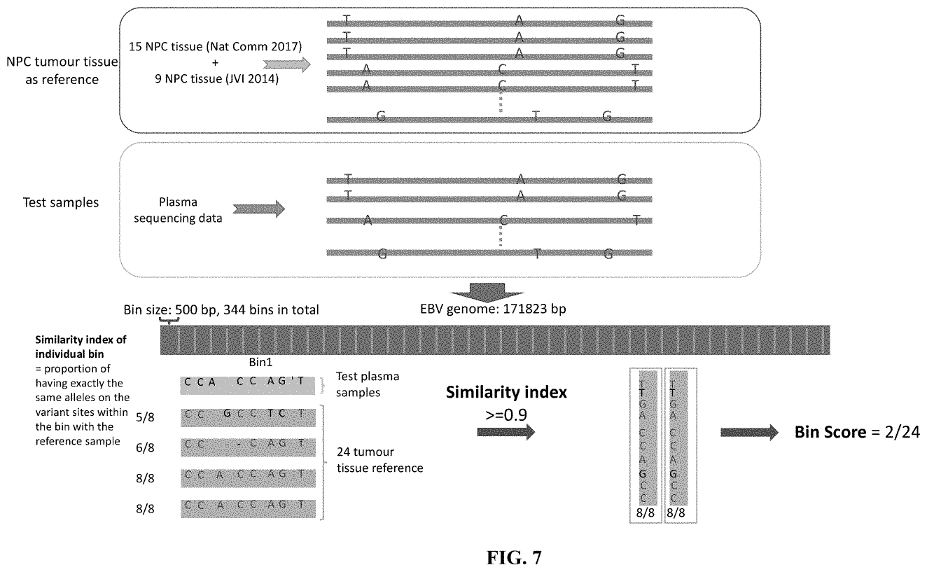

FIG. 7 illustrates the principle of block-based variant pattern analysis. Block-based analysis is used to evaluate the similarity of the EBV DNA variant patterns derived from the plasma EBV DNA sequencing of different samples to a reference genome and here the NPC sequencing data available in the public database (Kwok et al. J Virol 2014; 88:10662-72, Li et al. Nat Comm 2017; 8:14121) is used as a reference. In the block-based analysis, the EBV genome is divided into bins of 500 bp in size (344 bins in total) and the similarity of variant patterns of each bin with the 24 NPC samples in the reference set was compared. As an example, if there are 8 variant sites within one particular bin, the alleles on these sites within this bin of the test sample are analyzed and compared to the alleles on the same sites of the 24 reference samples. A similarity index is derived based on the proportion of having exactly the same alleles with the reference samples. For example, if the test sample has exactly the same alleles on 7 out of 8 variant sites with one reference sample, the similarity index of that bin would be 7/8 with that reference sample. And there would be 24 similarity indices of that bin of the test sample with comparison to the 24 reference samples. Based on the 24 similarity indices of that bin, a bin score is calculated which represents the overall similarity of variant patterns with the reference samples. For example, if the cutoff of similarity index is set at 0.9, the bin score counts the proportion of bins with indices higher than the cutoff. Hence, if there are only two out of 24 similarity indices higher than 0.9, the bin score is 2/24. The higher the bin score, the more similar the variant pattern of the test sample is to the reference sample set.

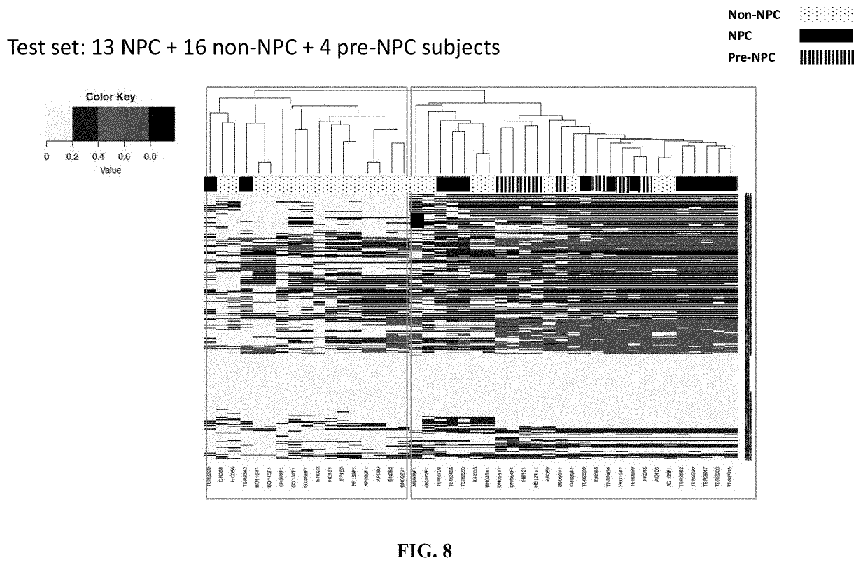

FIG. 8 shows block-based analysis of EBV DNA variant patterns of 13 NPC, 16 non-NPC and 4 pre-NPC samples. For each of the 4 pre-NPC subjects, samples from two time points were analyzed, hence giving a total of 8 subjects. The bin scores of the 344 bins of the EBV genome were derived for these samples. Based on the bin scores of these samples, unsupervised clustering analysis was performed. NPC samples (in black) were clustered together and non-NPC samples (marked with dots) were clustered together. The EBV variant profiles of pre-NPC subjects were clustered together with those of NPC subjects. Notably, the variant profiles of these 4 pre-NPC subjects were obtained through analysis of their baseline samples, which were collected years before the NPC development.

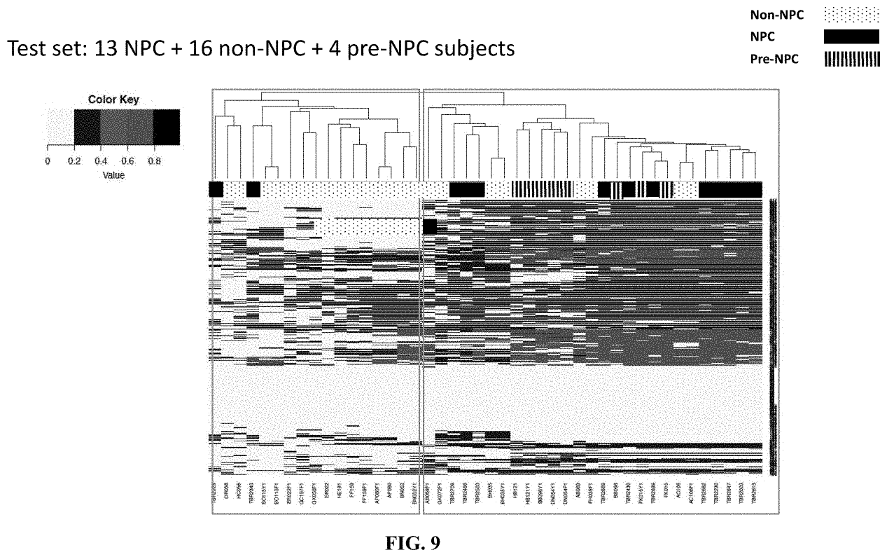

FIG. 9 shows block-based analysis of EBV DNA variants based on the EBV variants excluding the 29 variants reported in the study by Hui et al ((Hui et al. Int J Cancer 2019, doi.org/10.1002/ijc.32049) of the same group of 13 NPC, 16 non-NPC and 4 pre-NPC subjects. Similarly, the clustering of NPC samples (in black) was observed. Also, the EBV variant profiles of pre-NPC subjects were clustered together with those of NPC subjects. The clustering of the pre-NPC and NPC samples indicate that the variant analysis can predict the future development of NPC. In summary, the data in Example 3 and Example 4 reveal that those subjects who did not have NPC at recruitment but later developed the cancer had an EBV variant pattern in the baseline blood samples similar to those from other NPC patients.

### Example 5. Risk Prediction for NPC Using a Mathematic Model

This example describes construction of a classification model to predict the risk of future NPC development for subjects with detectable plasma EBV DNA using the analysis of the variant patterns, and the test results using the classification model.

A support vector machine (SVM) algorithm was used to construct a classifier using a training dataset compromising of 18 subjects without NPC and 8 NPC patients as described in Example 4. The testing dataset consisted of 5 NPC patients, 5 subjects without NPC and 8 samples collected from 4 subjects who did not have detectable NPC by endoscopy and MRI at the time of sample collection but were subsequently diagnosed of NPC (labelled as pre-NPC) as described in Example 4.

The method for the SVM analysis is described as follow:
Given a training dataset comprising n samples:

(M1,Y1), . . . ,(Mn,Yn)
where Yi indicates the NPC status of sample i. Yi is 1 for a sample from a NPC patient) or −1 for a sample from a subject without NPC; Mi is a p-dimensional vector comprising the viral variant patterns for a sample i. For example, Mi can be a series of variant sites such as 29 variants associated with NPC. Alternatively, Mi can be a series of block-based variant similarity scores (e.g., a non-overlapping windows of 500 bp) with respect to the reference EBV variants present in subjects known to have NPC.

A “hyperplane” was to be identified that separates the non-NPC and NPC groups as accurate as possible in a training dataset, by looking for a set of coefficients (W with p-dimensional vector) satisfying:

W·Mᵢ−b≥1 (for any subject in the NPC group) Criterion 1:

and

W·Mᵢ−b≤−1 (for any subject in the non-NPC group) Criterion 2
where W is a p-dimensional vector of coefficients determining the hyperplane; M is a matrix (p×n dimensions) with p variants (or block-based similarity scores) and n samples; b is the intercept.

The two criteria (i.e. criteria 1 and 2) can also be written as:

Yi(W*Mi−b)≥1 (criterion 3)

where Yi is either −1 (non-NPC) or 1 (NPC).

The margin distance (D) between criteria 1 and 2 is:

$$\frac{2}{‖ W ‖} ,$$

where ∥W∥ is computed using the distance from a point to a plane equation.

D is to be maximized by minimizing ∥W∥ subject to criterion 3.

Based on this principle, the parameters (W and b) of the classifier were determined. The NPC risk score for each of the test samples was then calculated by using the trained parameters (W and b).

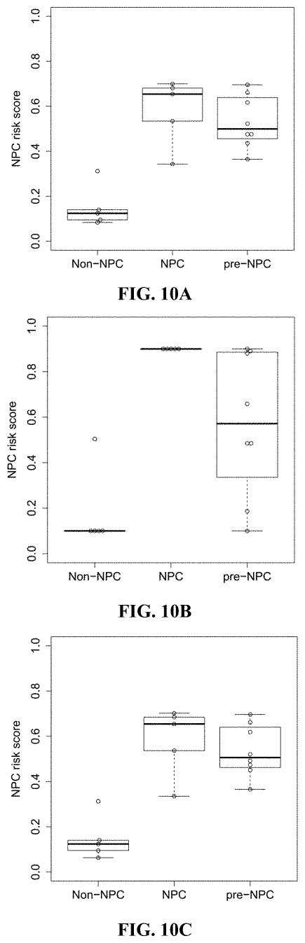

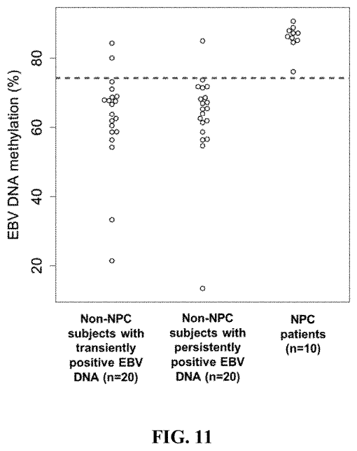

FIG. 10A shows the NPC risk score calculated using the trained classifier based on the analysis of all EBV variants using block-based variant analysis. For this analysis, the EBV genome was divided into 344 blocks of 500 bp for the calculation of bin score as described in Example 4. The bin score was considered as a feature for machine learning. The NPC risk scores of the NPC samples were significantly higher than those of the samples collected from the non-NPC subjects (mean NPC risk score: 0.15 vs 0.53, p-value <0.01, Student's t-test). Similarly, the NPC risk scores were significantly higher for the samples collected from the pre-NPC subjects compared with those without NPC (mean risk score: 0.58 vs 0.15, p-value <0.01, Student's t-test). Using a cutoff of 0.32, the samples from the NPC patients and the pre-NPC subjects could be differentiated from those without NPC with 100% sensitivity and 100% specificity.

FIG. 10B shows the NPC risk score calculated using the trained classifier based on the analysis of the 29 EBV variants reported in the study by Hui et al ((Hui et al. Int J Cancer 2019, doi.org/10.1002/ijc.32049). The NPC risk scores of the NPC samples were significantly higher than those of the samples collected from the non-NPC subjects (mean NPC risk score: 0.89 vs 0.18, p-value <0.01, Student's t-test). Similarly, the NPC risk scores were significantly higher for the samples collected from the pre-NPC subjects compared with those without NPC (mean risk score: 0.57 vs 0.18, p-value=0.02, Student's t-test). Using a cutoff of 0.6, the samples from the NPC patient and the pre-NPC subjects could be differentiated from those without NPC with 74% sensitivity and 100% specificity.

FIG. 10C shows the NPC risk score calculated using the trained classifier based on the analysis of all EBV variants using block-based variant analysis but excluding the 29 variants previously reported to be associated with NPC by Hui et al. (Hui et al. Int J Cancer 2019. doi: 10.1002/ijc.32049). The NPC risk scores of the NPC samples were significantly higher than those of the samples collected from the non-NPC subjects (mean NPC risk score: 0.58 vs 0.15, p-value <0.01, Student's t-test). Similarly, the NPC risk scores were significantly higher for the samples collected from the pre-NPC subjects compared with those without NPC (mean risk score: 0.53 vs 0.15, p-value <0.01, Student's t-test). Using a cutoff of 0.31, the samples from the NPC patient and those who subsequently developed NPC could be differentiated from those without NPC with 100% sensitivity and 100% specificity. These results indicate that the exclusion of the 29 previously reported EBV variants from the analysis would not adversely affect the accuracy of this analysis.

### Example 6. Analysis of Methylation Status of Plasma EBV DNA Via Bisulfite Sequencing

This example illustrates the use of bisulfite sequencing to differentiate the NPC patients and the non-NPC subjects but with detectable plasma EBV DNA based on the methylation status of plasma EBV DNA.

The methylation levels of EBV DNA in the plasma of NPC patients and subjects without NPC were determined using bisulfite sequencing. Bisulfite conversion can change unmethylated cytosine into uracil. Methylated cytosine cannot be altered by bisulfite and can remain as cytosine. During sequencing, the uracil can be determined as thymine. After sequencing, the methylation status of cytosines at any CpG dinucleotide context can be determined by checking if the cytosine has been changed to thymine.

The methylation levels of plasma EBV DNA were determined in 10 NPC patients and 40 subjects without cancer but with detectable EBV DNA in plasma (non-NPC subjects). For the 40 non-NPC subjects, another blood sample was collected from each of them 4 weeks later. Twenty of them became negative for plasma EBV DNA and they are labelled as having transiently positive plasma EBV DNA. Twenty of them remained positive for plasma EBV DNA and they are labelled as having persistently positive plasma EBV DNA.

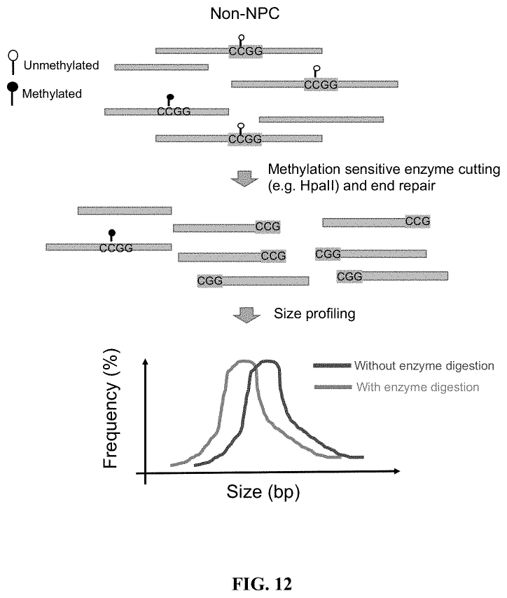

As shown in FIG. 11, the EBV DNA methylation level was significantly higher in the NPC patients compared with non-cancer subjects with transiently positive plasma EBV DNA (P<0.01, Student t-test) and non-cancer subjects with persistently positive plasma EBV DNA (P<0.01, Student t-test). These results suggest that the analysis of the methylation of the plasma EBV DNA can be useful for differentiating NPC patients and subjects without NPC but with detectable plasma EBV DNA.

### Example 7. Analysis of Methylation Status of Plasma EBV DNA Using Methylation-Sensitive Restriction Enzyme

This example describes an in-silico simulation experiment demonstrating the use of methylation-sensitive restriction enzyme analysis of plasma EBV DNA for differentiation of NPC patients and subjects without NPC but with detectable plasma EBV DNA.

Bisulfite sequencing of plasma DNA were performed with samples from a non-NPC subject and a NPC patient. 347,516 and 6,271,012 EBV DNA fragments in plasm DNA of the two subjects were obtained, respectively. The methylation levels of their plasma EBV DNA were 48.9% and 86.3%, respectively. It was determined that approximately half of the plasma EBV DNA molecules contained at least one “CCGG” motif.

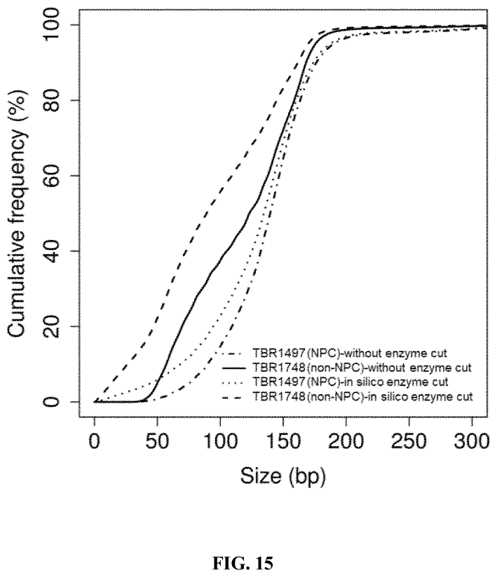

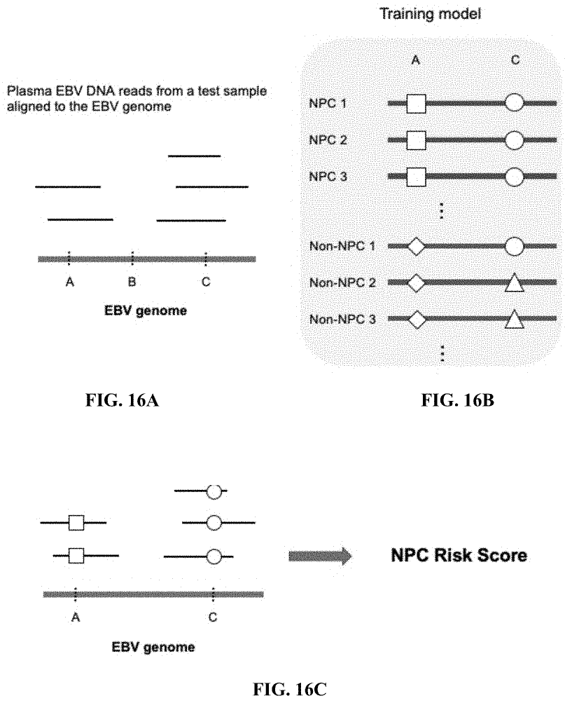

To simulate the restriction enzyme digestion on plasma EBV DNA, in-silico digestion of the plasma EBV DNA molecules was performed depending on their methylation statuses at “CCGG” sequence context inferred from bisulfite sequencing results. The simulated size profiles of plasma EBV DNA with and without in-silico digestion with methylation-sensitive restriction enzyme HpaII were thus obtained, as shown in FIG. 14. Without enzyme digestion, the size distribution of the plasma EBV DNA of the non-NPC subject was on the left side of that of the NPC subject, indicating that the size distribution was shorter for the non-NPC subject. This difference in fragment size was also observed in the size distribution profile with enzyme digestion, in that there was a significant increase in the abundance of short DNA of below 50 bp in the non-NPC subject with enzyme digestion as compared to without enzyme digestion. For the NPC patient, the proportions of the DNA molecules <50 bp were 5.87% and 0.84% for samples with and without enzyme digestion, respectively. For the non-NPC subject, however, the proportions of the DNA molecules of <50 bp were 22.24% and 4.99% for samples with and without enzyme digestion, respectively. The increase in the proportion of DNA of <50 bp on enzyme digestion were 17.2% and 5.0% for the NPC patient and non-NPC subject, respectively. FIG. 15 illustrates the cumulative size profiles of plasma EBV DNA with and without methylation-sensitive restriction enzyme digestion for a NPC patient and a non-NPC subject. The difference in the degree of enzyme digestion could be more easily appreciated using cumulative frequency curve against size. The gap between the two curves with and without enzyme digestion reflects the degree of digestion. The larger the gap, a larger degree the enzyme digestion made to the plasma EBV DNA, hence indicating a lower level of methylation in the plasma EBV DNA. As shown in the figure, the gap was larger for the non-NPC subject as compared with the NPC patient. The maximum distance between the curve without enzyme digestion and with enzyme digestion for the NPC patient and the non-NPC subject were 8.1 and 18.3, respectively; and the area between the two curves for the NPC patient and the non-NPC subject were 2395 and 942.9, respectively.

### Example 8. SNV Profile Analysis of Cell-Free EBV DNA Molecules

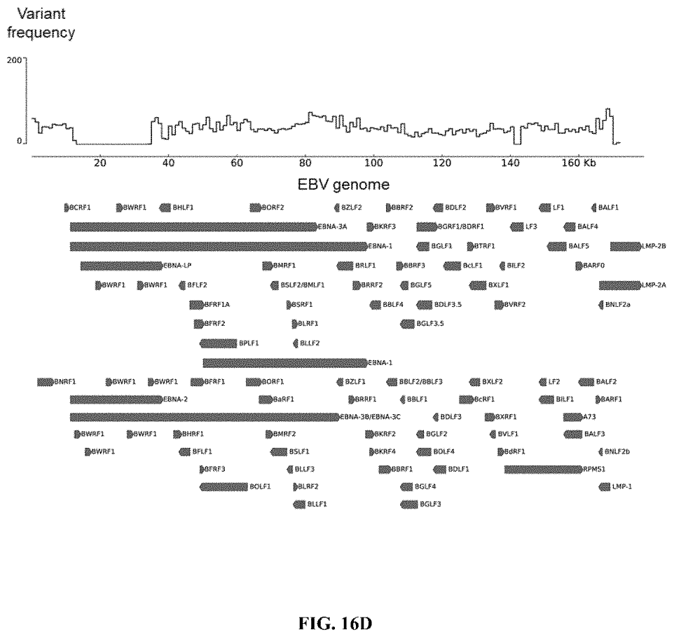

The difference in the EBV SNV profiles between two groups was analyzed in a training dataset which comprised plasma DNA sequencing data of 63 NPC and 88 non-NPC subjects. Differentiating SNVs across the EBV genome were identified. An NPC risk score was proposed to be derived from the genotypic patterns over these SNV sites, which was subsequently analyzed in a testing set of 31 NPC and 40 non-NPC samples. In this example, a total of 661 significant SNVs across the EBV genome were identified from the training set (FIG. 16D). In the testing set, NPC plasma samples were shown to have high NPC risk scores; there can be NPC-associated EBV SNV profiles. Among the non-NPC samples, there was a wide range of NPC risk scores. Non-NPC subjects can have diverse EBV SNV profiles.

Materials and Methods.

Study Participants and Design.

The study involved the analysis of a subset of the sequencing dataset of NPC and non-NPC plasma samples that was previously reported in Lam et al. Proc Natl Acad Sci USA. 2018; 115:E5115-E5124 (as the training set) and also newly sequenced plasma DNA samples from both NPC and non-NPC subjects (as the testing set).

The training dataset included plasma samples from both screen-detected NPC patients and non-NPC subjects in a previous prospective NPC screening study described in Lam et al. Proc Natl Acad Sci USA. 2018; 115:E5115-E5124. These non-NPC subjects harbored detectable levels of plasma EBV DNA by a real-time PCR-based assay. This dataset also included samples of symptomatic NPC patients from an independent cohort. The EBV genotypic information from the EBV isolates of all the samples was studied for building a training model for NPC risk score prediction. In this study, the plasma samples of another 31 symptomatic NPC patients and 40 non-NPC subjects were subject to target capture sequencing to serve as the testing set. These 31 symptomatic NPC patients were recruited from the Department of Clinical Oncology of the Prince of Wales Hospital, Hong Kong. The non-NPC subjects were also from the NPC screening cohort (including over 20,000 subjects) mentioned earlier and were randomly selected from it. The EBV genotypic variations from these NPC and non-NPC samples were analyzed, and their NPC risk scores were derived based on the training model. All NPC and non-NPC samples in the training and testing sets did not overlap.

Target Capture Sequencing.

Target capture sequencing of plasma samples was performed with enrichment of EBV DNA molecules from plasma DNA libraries through the capture-probe system (myBaits Custom Capture Panel, Arbor Biosciences). The EBV capture probes were designed to cover the entire viral genome. Probes which target 3,000 human single nucleotide polymorphism (SNP) sites were also included for reference. A probe mixture containing the molar ratio of EBV probes to autosomal DNA probes in the ratio of 100:1 was used in each capture reaction. DNA libraries from 10 plasma samples were multiplexed in one capture reaction, with equal amount of DNA libraries from each sample being used. The sequencing statistics for all the cases, including those previously reported cases used as the current training set, are stated in Tables 4A and 4B.

TABLE 4A
Sequencing statistics of all the NPC and non-NPC cases in
training set
TRAINING SET

| | | | No. of | | PCR |
|---|---|---|---|---|---|
| | | No. of raw | mapped | Mapping | duplication |
| Sample | Group** | fragments | fragments | rate (%) | rate (%) |
| GG017 | 0 | 32715321 | 30223262 | 92.4 | 43.1 |
| HL059 | 0 | 144554902 | 126762070 | 87.7 | 68.4 |
| DN045 | 0 | 78914933 | 68428310 | 86.7 | 66.9 |
| BP015 | 0 | 94168529 | 86145241 | 91.5 | 51.4 |
| AB126 | 0 | 56541949 | 54346856 | 96.1 | 24 |
| AC166 | 0 | 64450578 | 60439270 | 93.8 | 17.4 |
| AD092 | 0 | 71510547 | 69046150 | 96.5 | 16.1 |
| AE058 | 0 | 79728136 | 76825948 | 96.4 | 21.3 |
| AQ104 | 0 | 96938063 | 84743586 | 87.4 | 16.4 |
| BX011 | 0 | 72498952 | 70129591 | 96.7 | 14.9 |
| CA062 | 0 | 72180027 | 69744659 | 96.6 | 15.3 |
| CH131 | 0 | 71459860 | 68990753 | 96.5 | 22.2 |
| DC078 | 0 | 76239599 | 73238855 | 96.1 | 28.2 |
| DF038 | 0 | 100612788 | 97254251 | 96.7 | 26.1 |
| AG067 | 0 | 94932887 | 85387366 | 89.9 | 77.4 |
| AR027 | 0 | 61611288 | 59001573 | 95.8 | 15.1 |
| BL058 | 0 | 69559074 | 66513711 | 95.6 | 14.4 |
| AF118 | 0 | 64803996 | 61659065 | 95.2 | 14.4 |
| AF121 | 0 | 47656000 | 45104454 | 94.7 | 16 |
| AO097 | 0 | 64803246 | 62335332 | 96.2 | 14 |
| GV094 | 0 | 55594689 | 53398818 | 96 | 13.2 |
| AL092 | 0 | 88202778 | 84617253 | 95.9 | 20.7 |
| AM164 | 0 | 92235133 | 88753051 | 96.2 | 21.5 |
| EI030 | 0 | 67332747 | 64898723 | 96.4 | 13.7 |
| ER057 | 0 | 75611966 | 72851241 | 96.3 | 15.6 |
| FF077 | 0 | 88728791 | 84934257 | 95.7 | 18.3 |
| FF094 | 0 | 67950009 | 65456835 | 96.3 | 16.5 |
| AO100 | 0 | 74073437 | 71534001 | 96.6 | 14.4 |
| HE119 | 0 | 75939094 | 70594529 | 93 | 46.3 |
| GC110 | 0 | 109911126 | 101627813 | 92.5 | 30 |
| GT107 | 0 | 73134341 | 66124665 | 90.4 | 36.9 |
| GZ039 | 0 | 58128740 | 54517308 | 93.8 | 26.1 |
| AE151 | 0 | 118973652 | 109516490 | 92 | 21 |
| AH116 | 0 | 97765995 | 88477724 | 90.5 | 28 |
| AM095 | 0 | 87643692 | 80164284 | 91.5 | 19.6 |
| BP065 | 0 | 84740540 | 80067572 | 94.5 | 37.4 |
| EN086 | 0 | 32884093 | 31068440 | 94.5 | 38.3 |
| GC038 | 0 | 52719658 | 49985247 | 94.8 | 38.1 |
| AC106 | 0 | 46473277 | 43990963 | 94.7 | 82.5 |
| AP080 | 0 | 38659615 | 36293332 | 93.9 | 60 |
| GT123 | 0 | 90634113 | 82011875 | 90.5 | 65.1 |
| AE011 | 0 | 64587311 | 59269827 | 91.8 | 49.2 |
| BV159 | 0 | 108366362 | 97270043 | 89.8 | 73.8 |
| CZ031 | 0 | 104890395 | 93619970 | 89.3 | 73.4 |
| AL071 | 0 | 35231149 | 32775649 | 93 | 74.6 |
| AL122 | 0 | 132811199 | 123757690 | 93.2 | 76.6 |
| AS079 | 0 | 33454154 | 31094045 | 93 | 74.3 |
| AX070 | 0 | 82769034 | 77118993 | 93.2 | 75.8 |
| DC125 | 0 | 82353895 | 76845022 | 93.3 | 64.2 |
| DO041 | 0 | 98527392 | 91944421 | 93.3 | 63 |
| DN037 | 0 | 73898976 | 66401716 | 89.8 | 69.3 |
| DN131 | 0 | 85896965 | 77109501 | 89.8 | 68.8 |
| DS050 | 0 | 97058938 | 87190650 | 89.8 | 68 |
| DZ071 | 0 | 130632583 | 117555933 | 90 | 67.8 |
| EH050 | 0 | 144211569 | 131747254 | 91.4 | 67.5 |
| DZ026 | 0 | 63577798 | 60575778 | 95.3 | 24.9 |
| HM142 | 0 | 74460599 | 71830670 | 96.5 | 28.9 |
| HN068 | 0 | 58569268 | 56499964 | 96.5 | 27.6 |
| HR120 | 0 | 78697168 | 75901684 | 96.5 | 28.7 |
| CD005 | 0 | 67185044 | 64398576 | 95.8 | 18.9 |
| DC146 | 0 | 67286289 | 64869690 | 96.4 | 20.4 |
| DD090 | 0 | 72863832 | 69973561 | 96 | 18.9 |
| DE103 | 0 | 74532024 | 71748839 | 96.3 | 20.1 |
| DF112 | 0 | 80285807 | 77313233 | 96.3 | 16.6 |
| DH045 | 0 | 73283371 | 70644621 | 96.4 | 21 |
| DK016 | 0 | 98640353 | 95198449 | 96.5 | 22.8 |
| DK057 | 0 | 65024042 | 62488386 | 96.1 | 19.8 |
| DL055 | 0 | 64127942 | 61316770 | 95.6 | 18.9 |
| CE144 | 0 | 55972062 | 53546313 | 95.7 | 15.4 |
| CP042 | 0 | 67609649 | 64706108 | 95.7 | 15.2 |
| CZ046 | 0 | 55236628 | 52985764 | 95.9 | 13.5 |
| AP047 | 0 | 73544542 | 70437730 | 95.8 | 19.9 |
| AS108 | 0 | 74546824 | 71474684 | 95.9 | 22.1 |
| BF137 | 0 | 87739825 | 83608642 | 95.3 | 19.2 |
| AG020 | 0 | 67573799 | 63087296 | 93.4 | 17.6 |
| AE055 | 0 | 62308055 | 59551554 | 95.6 | 11.4 |
| AE105 | 0 | 59317164 | 56861140 | 95.9 | 10.2 |
| AE107 | 0 | 69376388 | 66837992 | 96.3 | 13.3 |
| AB004 | 0 | 69373853 | 66823399 | 96.3 | 12.4 |
| AC153 | 0 | 83546018 | 80433313 | 96.3 | 13.4 |
| AE026 | 0 | 80236204 | 77227885 | 96.2 | 13.8 |
| AF091 | 0 | 79865448 | 76665569 | 96 | 12.4 |
| HF020 | 0 | 73890276 | 69898875 | 94.6 | 11.9 |
| BO049 | 0 | 54341974 | 49518640 | 91.1 | 12.2 |
| CV094 | 0 | 69353920 | 62090890 | 89.5 | 11.9 |
| DM146 | 0 | 86198122 | 83306628 | 96.7 | 13.7 |
| DN054 | 0 | 57906125 | 55516552 | 95.9 | 21.6 |
| DN092 | 0 | 65436665 | 62867803 | 96.1 | 16.7 |
| AC173 | 1 | 77221448 | 69636427 | 90.2 | 53.5 |
| AO050 | 1 | 94201867 | 84771216 | 90 | 51.9 |
| AQ014 | 1 | 64826863 | 58371226 | 90 | 47.2 |
| AZ118 | 1 | 75307129 | 67827313 | 90.1 | 47.7 |
| AC088 | 1 | 76597786 | 55250665 | 72.1 | 47.2 |
| AL038 | 1 | 76499430 | 55322894 | 72.3 | 45.7 |
| AM086 | 1 | 84280496 | 61284379 | 72.7 | 43.4 |
| AT038 | 1 | 64157394 | 46063166 | 71.8 | 45.8 |
| BK041 | 1 | 61505610 | 44247376 | 71.9 | 44.8 |
| CF028 | 1 | 97748094 | 88104244 | 90.1 | 59.1 |
| CH047 | 1 | 123975141 | 112556783 | 90.8 | 56.6 |
| CL037 | 1 | 106862473 | 96469537 | 90.3 | 60.7 |
| CP006 | 1 | 61469649 | 54366171 | 88.4 | 59.4 |
| CD007 | 1 | 103710165 | 93643893 | 90.3 | 61.9 |
| DF120 | 1 | 96451355 | 89089726 | 92.4 | 51.6 |
| DH101 | 1 | 73023724 | 67311149 | 92.2 | 60.3 |
| EG016 | 1 | 83087673 | 77307393 | 93 | 24.2 |
| EN070 | 1 | 35732253 | 32582501 | 91.2 | 52.5 |
| EV013 | 1 | 70202729 | 64881793 | 92.4 | 35.8 |
| FD089 | 1 | 106149891 | 88230410 | 83.1 | 51.9 |
| FG092 | 1 | 58840935 | 54320095 | 92.3 | 36.8 |
| FM073 | 1 | 65062459 | 60232085 | 92.6 | 39.3 |
| FZ037 | 1 | 46211337 | 42733248 | 92.5 | 37.6 |
| GC137 | 1 | 73772882 | 68339539 | 92.6 | 62.9 |
| GS059 | 1 | 103768139 | 95756898 | 92.3 | 64.4 |
| GX170 | 1 | 112376826 | 104300963 | 92.8 | 60.7 |
| HD083 | 1 | 80146546 | 74256782 | 92.7 | 59.8 |
| HM169 | 1 | 69203940 | 64144652 | 92.7 | 59.7 |
| AG006 | 1 | 73346449 | 68476847 | 93.4 | 22.9 |
| FD163 | 1 | 62554476 | 58856976 | 94.1 | 27.7 |
| CX027 | 1 | 88012245 | 80202542 | 91.1 | 67.7 |
| CV009 | 1 | 60922871 | 56232165 | 92.3 | 45.6 |
| TBR1433 | 2 | 77708246 | 70039392 | 90.1 | 30.2 |
| TBR1470 | 2 | 73941394 | 67495510 | 91.3 | 21.6 |
| TBR1572 | 2 | 71106989 | 64814893 | 91.2 | 23.6 |
| TBR1605 | 2 | 115061297 | 94605333 | 82.2 | 47.8 |
| TBR1606 | 2 | 60654197 | 55309308 | 91.2 | 32 |
| TBR1607 | 2 | 75439582 | 69608132 | 92.3 | 28.1 |
| TBR1650 | 2 | 83518964 | 76881089 | 92 | 21.8 |
| TBR1665 | 2 | 73581524 | 68005926 | 92.4 | 26.7 |
| TBR1685 | 2 | 64858923 | 59295059 | 91.4 | 28.4 |
| TBR1794 | 2 | 77616481 | 72400504 | 93.3 | 31.9 |
| TBR1795 | 2 | 84087680 | 78757703 | 93.7 | 25.2 |
| TBR1821 | 2 | 89364373 | 83561953 | 93.5 | 25.2 |
| TBR1822 | 2 | 74207438 | 69089332 | 93.1 | 32.3 |
| TBR1841 | 2 | 76709226 | 71246483 | 92.9 | 27.6 |
| TBR1857 | 2 | 93499651 | 85084161 | 91 | 29.1 |
| TBR1911 | 2 | 102778437 | 93039420 | 90.5 | 28.3 |
| TBR1937 | 2 | 108092562 | 98448107 | 91.1 | 31.5 |
| TBR1950 | 2 | 100931791 | 92237772 | 91.4 | 31.7 |
| TBR1961 | 2 | 120837880 | 110269912 | 91.2 | 23.3 |
| TBR2032 | 2 | 74713097 | 70057803 | 93.8 | 27.1 |
| TBR2044 | 2 | 74572414 | 69808426 | 93.6 | 21.7 |
| TBR2059 | 2 | 68180154 | 63969165 | 93.8 | 22.8 |
| TBR2066 | 2 | 71590556 | 67039888 | 93.6 | 24.7 |
| TBR2129 | 2 | 67520639 | 63360453 | 93.8 | 22.9 |
| TBR1344 | 2 | 89830107 | 79295024 | 88.3 | 35.2 |
| TBR1358 | 2 | 37407353 | 35051007 | 93.7 | 41.9 |
| TBR1360 | 2 | 73282234 | 61715512 | 84.2 | 49.8 |
| TBR1378 | 2 | 54841088 | 50538475 | 92.2 | 34.5 |
| TBR1379 | 2 | 61335101 | 51046779 | 83.2 | 48.6 |
| TBR1390 | 2 | 50153930 | 44313840 | 88.4 | 45 |
| TBR1557 | 2 | 35803478 | 32801152 | 91.6 | 43.1 |

**group 0 = non-NPC subjects, group 1 = NPC subjects (Screening cohort), group 2 = NPC (External cohort).

TABLE 4B
Sequencing statistics of all the NPC and non-NPC cases in testing set
TESTING SET

| | | No. of | No. of | | PCR | NPC |
|---|---|---|---|---|---|---|
| | | raw | mapped | Mapping | duplication | risk |
| Sample | Group## | fragments | fragments | rate (%) | rate (%) | score |
| AB069 | 0 | 62333414 | 56996119 | 91.4375 | 67.0529 | 0.25 |
| AG102 | 0 | 50527076 | 47272142 | 93.558 | 79.7162 | 1.00 |
| BF034 | 0 | 30900262 | 29069989 | 94.0768 | 79.9262 | 0.06 |
| BH035 | 0 | 27968166 | 25683364 | 91.8307 | 78.2321 | 1.00 |
| BM060 | 0 | 44571256 | 41656811 | 93.4612 | 82.7252 | 1.00 |
| BN052 | 0 | 32654549 | 30177844 | 92.4154 | 77.7825 | 0.00 |
| BO115 | 0 | 20605498 | 18891596 | 91.6823 | 76.3716 | 0.00 |
| BR067 | 0 | 35222869 | 31942475 | 90.6867 | 10.9972 | 1.00 |
| BS030 | 0 | 29488585 | 26961246 | 91.4294 | 66.5338 | 0.99 |
| CB025 | 0 | 35335207 | 32498897 | 91.9731 | 81.8117 | 1.00 |
| CI095 | 0 | 44920271 | 41857137 | 93.181 | 64.8167 | 0.00 |
| CO003 | 0 | 22618823 | 20545705 | 90.8345 | 66.4679 | 1.00 |
| DK129 | 0 | 26650610 | 24552495 | 92.1273 | 66.7223 | 1.00 |
| DM162 | 0 | 46869923 | 42223785 | 90.0872 | 65.1806 | 0.99 |
| DO001 | 0 | 35030693 | 32412652 | 92.5264 | 64.0082 | 1.00 |
| DR058 | 0 | 33151251 | 30641021 | 92.4279 | 77.5861 | 0.41 |
| DX145 | 0 | 30538948 | 28353858 | 92.8449 | 64.0698 | 0.00 |
| DZ091 | 0 | 48775427 | 45509608 | 93.3044 | 79.647 | 0.00 |
| EB064 | 0 | 15486333 | 14294637 | 92.3049 | 77.2137 | 0.52 |
| EC056 | 0 | 44264275 | 41421171 | 93.577 | 64.8678 | 0.28 |
| EI052 | 0 | 30414618 | 28373013 | 93.2874 | 79.4382 | 0.98 |
| ER022 | 0 | 29318005 | 25814308 | 88.0493 | 64.2827 | 0.00 |
| ET022 | 0 | 28303377 | 26549950 | 93.8049 | 79.5254 | 0.97 |
| EZ015 | 0 | 34114519 | 31826767 | 93.2939 | 79.4083 | 0.65 |
| FF159 | 0 | 27631827 | 25177560 | 91.118 | 66.2635 | 0.00 |
| FH039 | 0 | 25047700 | 23182787 | 92.5546 | 73.199 | 1.00 |
| FV078 | 0 | 59919758 | 55955981 | 93.3849 | 82.1063 | 1.00 |
| GC157 | 0 | 22988959 | 21147818 | 91.9912 | 72.2857 | 0.00 |
| GG040 | 0 | 58823944 | 53857823 | 91.5577 | 10.9781 | 0.14 |
| GK072 | 0 | 28087271 | 26012505 | 92.6131 | 72.1235 | 0.99 |
| GV071 | 0 | 30298816 | 27995522 | 92.3981 | 81.7554 | 1.00 |
| GX058 | 0 | 52901878 | 47527912 | 89.8416 | 72.5617 | 0.00 |
| GZ082 | 0 | 33025312 | 30743443 | 93.0905 | 76.508 | 0.00 |
| HB042 | 0 | 39832106 | 37486823 | 94.1121 | 79.7558 | 0.59 |
| HC056 | 0 | 27801939 | 25722722 | 92.5213 | 77.5543 | 0.80 |
| HE176 | 0 | 26672711 | 24740453 | 92.7557 | 65.5094 | 0.00 |
| HE181 | 0 | 20151536 | 18596587 | 92.2837 | 77.1676 | 0.00 |
| HF010 | 0 | 36767150 | 34443572 | 93.6803 | 83.3378 | 0.99 |
| HK068 | 0 | 24744347 | 22950199 | 92.7493 | 66.3875 | 0.02 |
| HN102 | 0 | 18847144 | 17418641 | 92.4206 | 66.0707 | 0.00 |
| p003704 | 1 | 24089077 | 22256290 | 92.3916 | 75.6729 | 1.00 |
| P100405 | 1 | 27917819 | 25958361 | 92.9813 | 76.6278 | 1.00 |
| P100742 | 1 | 33868828 | 31121633 | 91.8887 | 77.043 | 1.00 |
| P101161 | 1 | 22077183 | 20555644 | 93.1081 | 76.2116 | 1.00 |
| TBR2003 | 1 | 89502393 | 78014093 | 87.1643 | 67.8335 | 1.00 |
| TBR2197 | 1 | 49274726 | 46072820 | 93.5019 | 79.8709 | 1.00 |
| TBR2230 | 1 | 19463878 | 17991477 | 92.4352 | 77.7681 | 1.00 |
| TBR2239 | 1 | 40477218 | 37931905 | 93.7117 | 79.5694 | 1.00 |
| TBR2269 | 1 | 36732370 | 33345425 | 90.7794 | 10.8014 | 0.85 |
| TBR2329 | 1 | 102625376 | 87445869 | 85.2088 | 79.1855 | 0.99 |
| TBR2343 | 1 | 47646593 | 41027985 | 86.109 | 80.656 | 1.00 |
| TBR2330 | 1 | 36942083 | 33822640 | 91.5559 | 11.0708 | 0.00 |
| TBR2385 | 1 | 42000104 | 39181234 | 93.2884 | 81.8537 | 1.00 |
| TBR2406 | 1 | 66799222 | 60524426 | 90.6065 | 83.3811 | 0.00 |
| TBR2430 | 1 | 19062836 | 17515880 | 91.885 | 77.2878 | 1.00 |
| TBR2466 | 1 | 39167493 | 35820959 | 91.4558 | 66.6063 | 1.00 |
| TBR2553 | 1 | 20976134 | 19085605 | 90.9872 | 78.5291 | 1.00 |
| TBR2605 | 1 | 28691106 | 26101695 | 90.9749 | 65.7645 | 1.00 |
| TBR2615 | 1 | 33489016 | 29864524 | 89.1771 | 68.4423 | 1.00 |
| TBR2641 | 1 | 113077610 | 94235991 | 83.3374 | 54.0705 | 0.98 |
| TBR2647 | 1 | 52926587 | 46699098 | 88.2337 | 68.1284 | 1.00 |
| TBR2655 | 1 | 44805097 | 41374955 | 92.3443 | 65.3989 | 1.00 |
| TBR2669 | 1 | 43399057 | 39819658 | 91.7524 | 65.4329 | 1.00 |
| TBR2682 | 1 | 35617499 | 32625124 | 91.5986 | 77.4284 | 1.00 |
| TBR2699 | 1 | 78986032 | 67322508 | 85.2334 | 80.332 | 1.00 |
| TBR2709 | 1 | 60912602 | 54630334 | 89.6864 | 78.8851 | 0.97 |
| TBR2847 | 1 | 19610868 | 17657654 | 90.0401 | 52.1991 | 1.00 |
| TBR2849 | 1 | 15220276 | 14043817 | 92.2704 | 51.0899 | 1.00 |
| TBR2868 | 1 | 21065832 | 18609241 | 88.3385 | 53.7439 | 1.00 |
| TBR2892 | 1 | 17905000 | 16600383 | 92.7137 | 51.5529 | 1.00 |
| TBR2906 | 1 | 29385280 | 26298916 | 89.4969 | 53.0486 | 1.00 |

##group 0 = non-NPC subjects, group 1 = NPC subjects

EBV Variant Calling.

Sequenced reads were aligned to the human (hg19) and EBV reference genome (AJ507799.2)) using the BWA aligner that is described in Li H et al. Bioinformatics 2010; 26:589-95, which is incorporated herein by reference in its entirety. An EBV single nucleotide variant (SNV) was identified with Samtools, as described in Li H et al. Bioinformatics. 2009; 25:2078-9, which is incorporated herein by reference in its entirety, when an alternative allele different from the reference viral genome over an EBV genomic site was detected. A SNV site with more than 1 type of allele detected (minor allele frequency cutoff set at 5%) was filtered out for the subsequent NPC risk score analysis.

NPC Risk Score.

In this example, the NPC risk score was the weighted summation of EBV genotypes at a fixed set of SNV sites across the viral genome (as explanatory variables in a binary logistic regression model). A set of NPC-associated SNVs was first identified by analyzing the difference in the EBV SNV profiles from NPC and non-NPC samples in the training set. The association of each variant across the EBV genome with the NPC cases were analyzed using the Fisher's exact test. Then a fixed set of significant SNVs were obtained with the false discovery rate (FDR) controlled at 5%.

The NPC risk score of a test sample can be determined by its EBV genotypes over this specific set of significant SNV sites identified from the training set. As mentioned, due to the low concentrations of plasma EBV DNA molecules, there might be incomplete coverage of the whole EBV genome by sequenced EBV DNA reads. The score was therefore formulated to be determined by the genotypic patterns over those SNV sites which were covered by plasma EBV DNA reads (e.g., with available genotypic information) (FIGS. 16A, 16B, and 16C). To derive the NPC risk score, the subset of significant SNV sites was first identified, which were covered by plasma EBV DNA reads in the test sample. Then, the weighting (effect sizes) of genotypes at each site was determined within the subset of significant SNV sites. This was done by analyzing the genotypic patterns at each site among the NPC and non-NPC samples in the training dataset (FIG. 16B). Based on this, a logistic regression model was constructed to inform the effect sizes of the risk genotypes at each SNV site on NPC. The logistic model was written as follow:

$$P = \frac{1}{1 + {e}^{- ( {β}_{0} + {\sum }_{k = 1}^{n} {β}_{k} {X}_{k} )}}$$

which could be rewritten as:

$$logit ( P ) = log ( \frac{P}{1 - P} ) = {β}_{0} + {Σ}_{k = 1}^{n} {β}_{k} {X}_{k} ,$$

where n is the number of significant SNV sites; β₀ and βₖ are the coefficients which could be determined by maximum likelihood estimator; P is the probability of the EBV-positive patient having NPC; the variable Xₖ represents the SNV site at genomic position k. Xₖ was coded as −1, if a variant present in a sample identical to the EBV reference genome. Xₖ was coded as 1, if an alternative variant present in a sample. Xₖ was coded as 0, if the analyzed variant site was not covered in a sample. ‘LogisticRegression’ function (penalty=‘l2’, C=1, solver=‘saga’, max_iter=5000, and random_state=0) was used in python to estimate the coefficients β₀ and βₖ. This was done by analyzing the genotypic patterns at each site among the NPC and non-NPC samples in the training dataset. A matrix (c+d)×n was fed into the python, where c was the number of NPC samples, d was the number of non-NPC samples in the training set, and n was the number of genotypic variants. Each row represented a sample (0 for a patient without NPC; 1 for a patient with NPC), and each column represented a variant. Then the coefficients (β₀ and βₖ) could be deduced. The NPC risk score of the test sample was then derived based on its own genotypes at SNV sites, weighted by the corresponding coefficients β₀ and βₖ deduced from the training model. (FIG. 16C).

Results

Building the NPC Risk Score Training Model.

As mentioned above, previously reported plasma EBV DNA sequencing data of NPC and non-NPC samples were used for the NPC risk score training model development. Target capture sequencing had been performed to enrich the EBV DNA in the plasma samples. The viral SNV profiles of EBV isolates from NPC and non-NPC samples were studied here. From this dataset, those NPC and non-NPC cases with at least 30% of coverage over the EBV genome by the sequenced EBV DNA reads were selected. This cutoff was selected because more than 95% of the NPC samples in the training dataset had the viral genome coverage greater than the cutoff (Tables 4A and 4B). The demographics of these selected NPC and non-NPC subjects, including the age and sex, and the cancer stage information (8th AJCC edition) of NPC patients are detailed in the Table 5. The sequencing statistics of these selected NPC and non-NPC samples are stated in the (Tables 4A and 4B).

TABLE 5
Subject characteristics of all the NPC and non-NPC cases in
the training set

| | NPC patients | Non-NPC subjects |
|---|---|---|
| Number | 63 | 88 |
| Sex | | |
| M | 56 | 88 |
| F | 7 | 0 |
| Median age, year (IQR) | 53 (47.5-57.5) | 54 (48-59) |
| Tumor stage | | |
| I | 17 | NA (non-applicable) |
| II | 11 | NA |
| III | 26 | NA |
| IV | 9 | NA |

The EBV SNV profiles of these 63 NPC and 88 non-NPC samples were analyzed. The median sequencing depth over the EBV genome for all the samples was 2× (interquartile range (IQR), 1.0×-9.2×). The mean number of EBV SNVs identified from NPC samples was 800 (IQR, 662-958), and the mean number of SNVs among the non-NPC samples was 539 (range, 363-656). In total, there were 5678 different SNVs identified for all the samples. The distribution of these SNVs across the EBV genome was illustrated in the FIG. 16D.

The association of each viral SNV with NPC samples in the training set was also studied with Fisher's exact test. A total of 661 significant SNVs were identified which were associated with NPC with adjusted p-values by controlling a false discovery rate (FDR) at 0.05. The genomic location of these 661 SNVs are listed in Table 6. Subsequently the NPC risk scores of the testing set of plasma samples of NPC and non-NPC subjects were derived based on the genotypic patterns over these 661 SNV sites.

TABLE 6
EBV Genomic Locations (relative to AJ507799.2) of 661 Exemplary SNVs

EBV genomic positions
46, 156, 158, 206, 212, 246, 390, 409, 475, 505, 536, 570, 612, 628, 631, 866, 1067, 1072,
1074, 1133, 1137, 1176, 1194, 1195, 1322, 1349, 1373, 1384, 1391, 1534, 1875, 1992, 2709,
2772, 3223, 3379, 3820, 3941, 4863, 5398, 5745, 5802, 5849, 6066, 6108, 6209, 6287, 6379,
6483, 6555, 6583, 6865, 6883, 6885, 6910, 6943, 6998, 7000, 7015, 7047, 7133, 7188, 7208,
7212, 7232, 7246, 7261, 7296, 7326, 7356, 7385, 8233, 8344, 8455, 8567, 8872, 10623,
11323, 11694, 35308, 35492, 35526, 35550, 35583, 35615, 35637, 35678, 35856, 35869,
35974, 36067, 36166, 36577, 36667, 36694, 36768, 36798, 36847, 36948, 36950, 37051,
37053, 37284, 37465, 37624, 37641, 37671, 37682, 37701, 37739, 37834, 37954, 40549,
40555, 40835, 41153, 41402, 42209, 42321, 42422, 42712, 42948, 42992, 43088, 43235,
43280, 43312, 43396, 43419, 43611, 43806, 43819, 44122, 44530, 44650, 45100, 45616,
45691, 45694, 45823, 46105, 46133, 46610, 46895, 47904, 48633, 48730, 48997, 50133,
50754, 50764, 50881, 50946, 51080, 51151, 51152, 51227, 51269, 51379, 51435, 51514,
51517, 51588, 51847, 52549, 53683, 57411, 58192, 58207, 59205, 59334, 59390, 59435,
59489, 59588, 60005, 60239, 60453, 60887, 60893, 61256, 62141, 62456, 62499, 62509,
62741, 62819, 63302, 63911, 64131, 64171, 64216, 64234, 64882, 64921, 65465, 66364,
66434, 66718, 66749, 66961, 67054, 67621, 67721, 67745, 67867, 68260, 68303, 68304,
68509, 68885, 69483, 75030, 75287, 75326, 76761, 76917, 77195, 77815, 77816, 78662,
79264, 79318, 79649, 79739, 80313, 80349, 80609, 80626, 80635, 80840, 80919, 80978,
81110, 81212, 81682, 81722, 82332, 82369, 83062, 83639, 84127, 84257, 84345, 84390,
84413, 84524, 84739, 84766, 84799, 84883, 84887, 84917, 84970, 85076, 85125, 85128,
85224, 85227, 85228, 85801, 85840, 86113, 86779, 86794, 87397, 87556, 88012, 88121,
88223, 88303, 88464, 88500, 88552, 88597, 88636, 88837, 88900, 89630, 89819, 89850,
89920, 90477, 90553, 90585, 90641, 91005, 91011, 91046, 91179, 91429, 91430, 91437,
91765, 93097, 93367, 93468, 94793, 95291, 95379, 95458, 95509, 95631, 98147, 98243,
98261, 98376, 98489, 98841, 98984, 98985, 99057, 99069, 99329, 99350, 99355, 99736,
99760, 99805, 100552, 101509, 101691, 101920, 101986, 102922, 103333, 103824, 104286,
104432, 104549, 104554, 104672, 104804, 105670, 106006, 106374, 106468, 107457,
107592, 108012, 108332, 108351, 108355, 108419, 109234, 109507, 109576, 109775,
109939, 110032, 110477, 110687, 110773, 110873, 110939, 111026, 111694, 112486,
112980, 113691, 113718, 114468, 114762, 114811, 115371, 115462, 115574, 115639,
115711, 115726, 116058, 116310, 116393, 116394, 116501, 116583, 116807, 117030,
117291, 117456, 117564, 117994, 118097, 118210, 118349, 118432, 118460, 118505,
118955, 119031, 119295, 119381, 119417, 119786, 119804, 120294, 120318, 120360,
120672, 120866, 121160, 121164, 121230, 121383, 121473, 121689, 121719, 121737,
121776, 121893, 122140, 122208, 122340, 122343, 122361, 122443, 122481, 122490,
122607, 122610, 122820, 123174, 123312, 124938, 125271, 126135, 126225, 126442,
126601, 126681, 127197, 127408, 127465, 127597, 127615, 127840, 127991, 128036,
128268, 129730, 129835, 129904, 130450, 130453, 130687, 132047, 132182, 132224,
133635, 133648, 133779, 133947, 134155, 134157, 134199, 134349, 134371, 134385,
134718, 134729, 134760, 134766, 134788, 134874, 135060, 135078, 135102, 135108,
135117, 135354, 135606, 135866, 135949, 136053, 136077, 136185, 136554, 136645,
136914, 136932, 136974, 137080, 137142, 137315, 137346, 137480, 138869, 139209,
139440, 139495, 139683, 139945, 140001, 140059, 140227, 140254, 140256, 140305,
140492, 140569, 140600, 140688, 140744, 143451, 144072, 144086, 144354, 144564,
144684, 145144, 145245, 145538, 145736, 145918, 146158, 146237, 146241, 146242,
146249, 146270, 146557, 146627, 146690, 146744, 146756, 146764, 146887, 147059,
147060, 147068, 147088, 147102, 147310, 147426, 147478, 147492, 147607, 147651,
147663, 147681, 147698, 147708, 147731, 147773, 147783, 147849, 147882, 147899,
148050, 148230, 148283, 148488, 148627, 148636, 148930, 148971, 149130, 149318,
149354, 149643, 149835, 149925, 150021, 150027, 150171, 150356, 150470, 150749,
150777, 151139, 151146, 151202, 151255, 151337, 151352, 151370, 151643, 151821,
151876, 151942, 152023, 152086, 152244, 152611, 152945, 152946, 153011, 154386,
154614, 154971, 155084, 155388, 155390, 155608, 155919, 155988, 156012, 156132,
156138, 156153, 156183, 156282, 156636, 156695, 156797, 156809, 156818, 157052,
157124, 157229, 157427, 157466, 157805, 157823, 158015, 158142, 158407, 158429,
158480, 158777, 159219, 160803, 160826, 160970, 161035, 162116, 162146, 162194,
162214, 162236, 162463, 162475, 162506, 162851, 163106, 163286, 163292, 163363,
163403, 163421, 163463, 163610, 163628, 163685, 163925, 163994, 164723, 165086,
165850, 167201, 168172, 168176, 168411, 168432, 168466, 168559, 168593, 168596,
168659, 169008, 169428

Evaluation of the NPC Risk Score Training Model.

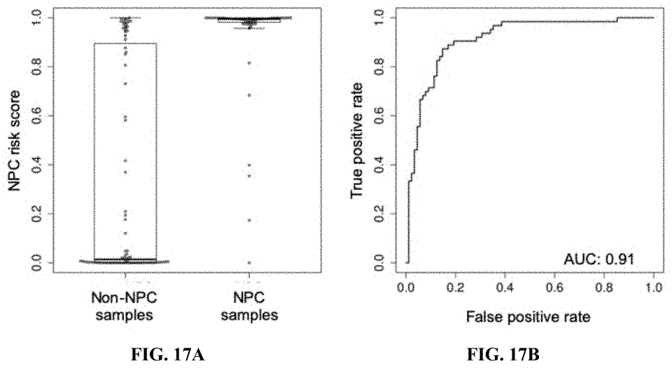

The training model was evaluated for analyzing the NPC risk scores of samples within the training set using the leave one-out approach. In the leave one-out approach, the principle of building the training model and deriving NPC risk score was the same as described in the Methods. All except one sample in the training set were used to build the training model and the one left out can be analyzed for its NPC risk score. In the leave one-out analysis, the median NPC risk score of the NPC group was 0.99 (IQR, 0.98-1.0) and that of the non-NPC group was 0.01 (IQR, 0.00-0.89) (FIG. 17A). Receiver operating characteristics (ROC) curve analysis was used to evaluate the differentiation of NPC and non-NPC samples by the NPC risk score. The area under the curve value was 0.91 (FIG. 17B).

NPC Risk Score Analysis in the Testing Set.

Target capture sequencing was performed on plasma samples of another 31 NPC patients and 45 non-NPC subjects. Among them all the 31 NPC samples and 40 non-NPC samples had at least 30% or more coverage of the EBV genome by the sequenced EBV DNA reads. The clinical characteristics of these NPC and non-NPC subjects are summarized in the Table 7. The sequencing statistics of this testing set of samples are also stated in the Tables 4A and 4B.

TABLE 7
Subject characteristics of all the NPC and non-NPC cases
in the testing set

| | NPC patients | Non-NPC subjects |
|---|---|---|
| Number | 31 | 40 |
| Sex | | |
| M | 26 | 40 |
| F | 5 | 0 |
| Median age, year (IQR) | 53 (47-61.5) | 53 (50-57) |
| Tumor stage | | |
| I | 6 | NA (not applicable) |
| II | 2 | NA |
| III | 12 | NA |
| IV | 11 | NA |

The NPC risk scores of the testing set of 31 NPC samples and 40 non-NPC samples based on the training model developed were analyzed. The NPC risk score of the sample can be determined by its variant patterns over the 661 significant SNV positions identified from the training set. Since there might be incomplete coverage of the EBV genome, only the SNV sites which were covered by the sequenced EBV DNA reads and had the corresponding allele information can be included in the NPC risk score analysis (FIGS. 16A, 16B, and 16C).

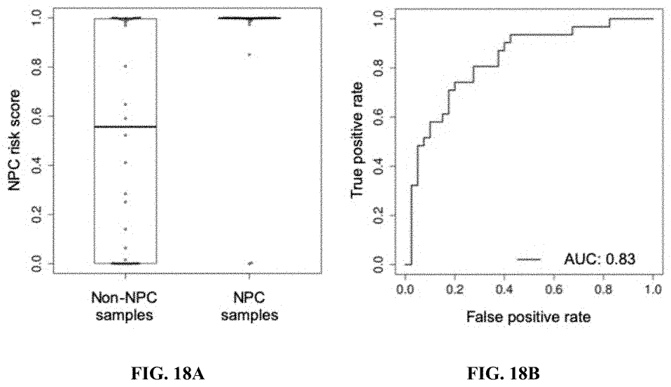

The median NPC risk score of the NPC group was 0.999 (IQR, 0.996-0.999) and that of the non-NPC group was 0.557 (IQR, 0.000-0.996) (FIG. 18A). Similarly, high NPC risk scores were noted among these 31 NPC samples. NPC samples in the testing set can share similar EBV SNV profiles with those NPC samples in the training set. The differentiation of NPC and non-NPC samples by the NPC risk score was also evaluated by ROC curve analysis. The area under the curve value was 0.83 (FIG. 18B).

Analysis of Genotypic Pattern over High-Risk Variant Sites in the Testing Set.

There are high-risk NPC-associated EBV variants in the EBER (EBV-encoded small RNA) region. In the EBER region, 23 significant SNVs had been reported by Hui et al. A similar approach of NPC risk prediction was adopted in the testing set of the 31 NPC and 40 non-NPC samples but based on only the genotypic patterns of the 23 reported SNVs in the EBER region were analyzed.

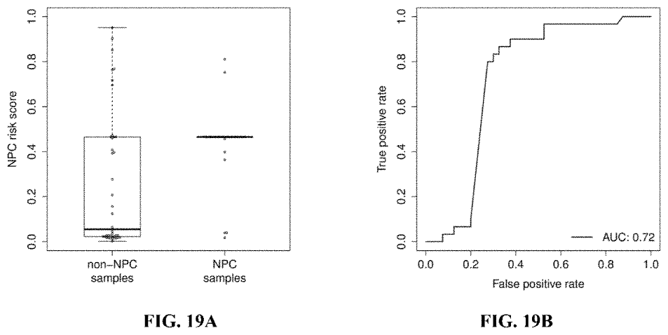

In the testing set, 31 out of the 71 NPC and non-NPC samples (44%) had EBV DNA reads covering all the 23 SNV sites. As shown in Table 8, for each of these 23 SNV sites, only a proportion of the samples had available genotypic information with reads covering the SNV sites (i.e. not all 23 SNV sites were covered with plasma EBV DNA reads in the samples). The percentages of the high-risk genotypes at each of the 23 SNV sites among the NPC samples range from 86% to 97%. The percentages of the high-risk genotypes among the non-NPC samples range from 35% to 52%. The numbers of NPC and non-NPC samples analyzed refer to the samples with available genotypic information (e.g., with EBV DNA reads covering the SNV sites). There were only a proportion of the samples in the testing set (31 NPC samples and 40 non-NPC samples) which had reads covering the SNV sites and available genotypic information over the corresponding sites. The differentiation of NPC and non-NPC samples was also evaluated by only analyzing the genotypic patterns of the 23 SNVs in the EBER region by ROC curve analysis. The area under the curve value was 0.72 (FIGS. 19A and 19B). This value was lower than that derived from the analysis of genotypic patterns over the whole EBV genome (0.83). Analysis of the genotypic patterns over the whole EBV genome can achieve better differentiation of NPC and non-NPC samples than that over a fixed viral genomic region.

TABLE 8
Genotypic patterns of NPC and non-NPC cases in the testing set
at the 23 SNV sites on the EBER gene

| | | No. of | No. of | No. of NPC | No. of non- |
|---|---|---|---|---|---|
| | | NPC | non-NPC | samples with | NPC samples |
| SNV | Risk | samples | samples | risk allele | with risk allele |
| position | allele | analyzed | analyzed | (Percentage) | (Percentage) |
| 5398 | A | 29 | 31 | 25 (86%) | 12 (39%) |
| 5849 | T | 28 | 27 | 24 (86%) | 11 (41%) |
| 6483 | T | 29 | 19 | 25 (86%) | 9 (47%) |
| 6583 | G | 29 | 16 | 25 (86%) | 7 (44%) |
| 6865 | A | 29 | 25 | 26 (90%) | 9 (36%) |
| 6883 | G | 29 | 25 | 27 (93%) | 11 (44%) |
| 6885 | T | 29 | 23 | 26 (90%) | 10 (43%) |
| 6910 | A | 29 | 23 | 26 (90%) | 8 (35%) |
| 6943 | G | 29 | 23 | 28 (97%) | 11 (48%) |
| 6998 | G | 30 | 26 | 29 (97%) | 11 (42%) |
| 7000 | T | 30 | 25 | 29 (97%) | 10 (40%) |
| 7011 | G | 30 | 26 | 29 (97%) | 11 (42%) |
| 7015 | T | 30 | 25 | 29 (97%) | 11 (44%) |
| 7047 | C | 30 | 29 | 29 (97%) | 14 (48%) |
| 7124 | G | 29 | 28 | 28 (97%) | 11 (39%) |
| 7133 | C | 29 | 28 | 28 (97%) | 12 (43%) |
| 7197 | T | 28 | 26 | 27 (96%) | 10 (38%) |
| 7205 | A | 28 | 26 | 27 (96%) | 11 (42%) |
| 7212 | C | 28 | 27 | 27 (96%) | 11 (41%) |
| 7232 | A | 29 | 28 | 25 (86%) | 11 (39%) |
| 7261 | A | 29 | 27 | 28 (97%) | 14 (52%) |
| 7296 | T | 28 | 26 | 27 (96%) | 13 (50%) |
| 7326 | C | 28 | 26 | 27 (96%) | 12 (46%) |

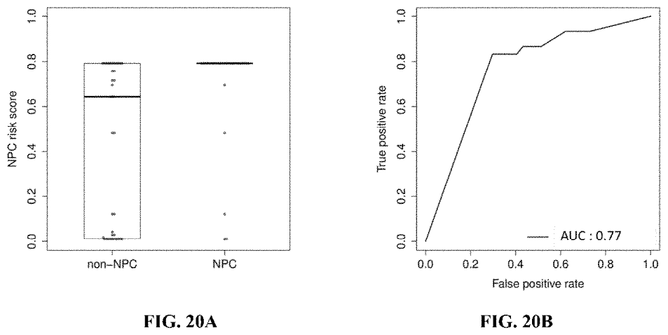

Similarly, 3 high-risk SNVs on the BALF2 (BamHI A left frame-2) gene have also been reported (Xu et al. Nat Genet. 2019; 51:1131-6). In the testing set, there were 55 out of the 71 samples (78%) which had EBV DNA reads covering all 3 SNVs. For each of these 3 SNV sites, only a proportion of the samples in the testing set had reads covering the SNV sites with available genotypic information (Table 9). The percentages of the high-risk genotypes at each of the 3 SNV sites among the NPC samples range from 86% to 93%. The percentages of the high-risk genotypes among the non-NPC samples range from 47% to 65%. There were 4 cases with no EBV DNA reads covering any of the 3 reported SNVs on the BALF2 gene (1 NPC and 3 non-NPC samples) and these cases could not be analyzed. A similar approach of NPC risk prediction was adopted in the remaining 30 NPC and 37 non-NPC samples from the testing set and only analyzed the genotypic patterns of the 3 SNVs reported in the BALF2 region. The differentiation of NPC and non-NPC samples was also evaluated by ROC curve analysis. The area under the curve value was 0.77 (FIGS. 20A and 20B). This value was lower than that derived from the analysis of genotypic patterns over the whole EBV genome (0.83). Analysis of the genotypic patterns over the whole EBV genome can achieve better differentiation of NPC and non-NPC samples than that over a fixed viral genomic region.

TABLE 9
Genotypic patterns of NPC and non-NPC cases in the testing set
at the 3 SNV sites on the BALF2 gene

| | | | | | No. of |
|---|---|---|---|---|---|
| | | | No. of | No. of NPC | non-NPC |
| | | No. of NPC | non-NPC | samples with | samples with |
| SNV | Risk | samples | samples | risk allele | risk allele |
| position | allele | analyzed | analyzed | (Percentage) | (Percentage) |
| 162214 | C | 30 | 31 | 28 (93%) | 20 (65%) |
| 162475 | C | 30 | 32 | 27 (90%) | 17 (53%) |
| 163363 | T | 29 | 32 | 25 (86%) | 15 (47%) |

The NPC risk score analysis described in this example allows for NPC risk prediction based on the genotypic patterns over a floating number of randomly selected SNVs within the set of 661 significant SNVs over the EBV genome (Table 6). A floating number of SNV sites used for NPC risk score analysis can be determined by whether the SNV sites were covered by the sequenced EBV DNA reads and had the corresponding allele information. Down-sampling of the set of 661 significant SNVs has been performed and the performance of the NPC prediction of the samples has been analyzed in the testing set using the same approach with the floating number of SNVs within the down-sampled set of SNVs. For the down-sampling analysis, a certain number (e.g., 23, 25, 100, 200, or 500) of SNVs were randomly selected from the 661 significant SNVs. Then, for a test sample, the SNV sites within the set of down-sampled SNVs that were covered by the EBV DNA sequence reads were identified. An NPC Risk Score Training Model was then obtained by training the model with the genotypic patterns of the NPC and non-NPC samples in the training set over the covered, down-sampled SNV sites. Through the training, the weighting of genotypes at each site was determined for the training model. The NPC risk score of a test sample was then derived by applying its own genotypic patterns over these covered, down-sampled SNV sites to the NPC Risk Score Training Model that was weighted over the same down-sampled SNV sites. The prediction performance of the NPC Risk Score Training Model with varying numbers of SNV sites is summarized in Table 10. For a given number of SNV sites, the down-sampling with random selection of SNVs was performed for 10 times, and the area under the curve value in the Table 10 was the average result among the 10 times of random down-sampling. The set of SNVs across the whole EBV genome were down-sampled to 23, which is the same as the number of the reported SNVs in the EBER region. The differentiation of NPC and non-NPC samples was evaluated by ROC curve analysis. The area under the curve value was 0.78. This value is higher than that with analysis of genotypic patterns of the 23 reported SNVs over EBER region (0.72).

TABLE 10
NPC prediction performance based on varying numbers of SNVs

| Number of down-sampled SNVs | Area under the curve (AUC) value |
|---|---|
| 23 | 0.78 |
| 25 | 0.78 |
| 100 | 0.77 |
| 200 | 0.83 |
| 500 | 0.79 |
| 661 | 0.83 |
| (all SNVs) | |

This study reports the analysis of EBV genotypic information through plasma DNA sequencing. Through paired-end sequencing, the differentiating molecular characteristics of plasma EBV DNA molecules were identified, including the count and size, between NPC and non-NPC subjects who harbored plasma EBV DNA. Incorporating such count and size-based analysis of plasma EBV DNA can almost double the positive predictive value of the current PCR-based protocol and this can form the basis of the second-generation sequencing-based screening test. Sequencing of plasma samples from NPC and non-NPC subjects can additionally yield EBV genotypic information and can enhance its potential clinical utility.

The NPC risk score can be used to be determined by the viral genome-wide markers instead of a single gene marker. Here the risk score was derived based on the variant patterns over the differentiating SNV sites across the EBV genome. Plasma sequencing for EBV genotypic information can involve sequencing plasma samples with a low concentration of EBV DNA molecules and therefore result in incomplete coverage of the EBV genome. In some cases, the informative SNV sites may not be covered by any EBV DNA reads, and in some cases it is not possible to tell if an individual carries a high-risk EBV strain type. This is supported by the result that, for each of the 23 reported SNV sites on the EBER gene, only some of the 71 analyzed samples in the testing set had reads covering the sites. The NPC samples in the testing set were shown to have high NPC risk scores, which can indicate the presence of NPC-associated EBV SNV profiles. Here the capture probe method was adopted for enrichment of EBV DNA molecules in plasma samples. An amplicon sequencing approach can also be used to enrich EBV DNA fragments which can target the high-risk variant regions for the genotypic information.

The genotypic patterns of the NPC and non-NPC samples in the testing set over the recently reported high-risk variant sites on the EBER gene and the BALF2 gene have been analyzed here. The distributions of high-risk genotypes in NPC and non-NPC samples are consistent with the results of the two studies which analyzed cellular samples, i.e. NPC tumor tissues and saliva samples of normal control subjects. Since all three studies including the current one were conducted in the same or neighboring localities within the southern parts of China, the distribution of EBV genotypes among normal control subjects can be similar. This provides evidence of the feasibility of EBV genotyping analysis through sequencing of plasma samples.

There can be clinical utility in profiling the EBV SNVs from plasma samples in the context of screening. As mentioned, approximately 5% of the screening population can harbor EBV DNA in plasma but do not have NPC (the false positive group). The data here revealed that these non-NPC subjects had variable NPC risk scores which can involve diverse EBV SNV profiles. There can exist a heterogenous group of individuals who had different risks of developing NPC in the future. Some of them who carried a high-risk EBV strain can have a higher future risk for NPC. The NPC risk score can be used to stratify those non-NPC subjects into different risk groups based on the viral genome-wide SNV profile. In one example, more frequent screening can be warranted for those with high NPC risk scores.

The EBV genotypic information from NPC patients and non-NPC subjects was analyzed through sequencing analysis of their plasma samples. While previous studies focused on identifying the high-risk variants associated with NPC on a population level, this study provides an insight on the clinical application of viral genotypic analysis. Such analysis can be used to inform the cancer risk on an individual basis by characterizing the EBV genotypes they harbor.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein can be employed in practicing the disclosure. It is intended that the following claims define the scope of the disclosure and that methods and structures within the scope of these claims and their equivalents be covered thereby.

1. A method of screening a pathogen-associated disorder in a subject, comprising:
a) receiving data from a first assay performed at a first time point that comprises determining a characteristic of cell-free nucleic acid molecules from a pathogen in a biological sample of the subject, wherein the characteristic of the cell-free nucleic acid molecules from the pathogen comprises amount, methylation status, variant pattern, fragment size, or relative abundance as compared to cell-free nucleic acid molecules from the subject in the biological sample, and wherein the characteristic indicates a risk for the subject to develop the pathogen-associated disorder; and
b) determining, based on the characteristic, a second time point at which a second assay is performed to screen for the pathogen-associated disorder in the subject, wherein an interval between the first time point and the second time point inversely correlates with the risk.

2. A method of prognosticating a pathogen-associated disorder in a subject, comprising:
receiving data from a first assay that comprises determining a characteristic of cell-free nucleic acid molecules from a pathogen in a biological sample of the subject, wherein the characteristic of the cell-free nucleic acid molecules from the pathogen comprises amount, methylation status, variant pattern, fragment size, or relative abundance as compared to cell-free nucleic acid molecules from the subject in the biological sample; and
generating a report indicative of a risk for the subject to develop the pathogen-associated disorder based on the characteristic of the cell-free nucleic acid molecules from the pathogen, and one or more factors of age of the subject, smoking habit of the subject, family history of the pathogen-associated disorder of the subject, genotypic factors of the subject, ethnicity of the subject, or dietary history of the subject.

3. The method of claim 1, wherein result of the first assay does not result in a medical treatment of the subject for the pathogen-associated disorder.

4. (canceled)

5. The method of claim 1, wherein the subject is diagnosed as not having the pathogen-associated disorder before the determining a second time point by a clinical diagnostic examination that has a false positive rate below 1%.

6. The method of claim 5, wherein the clinical diagnostic examination comprises physical examination, invasive biopsy, endoscopy, magnetic resonance imaging, positive emission tomography, computed tomography, or x-ray imaging.

7-10. (canceled)

11. The method of claim 1, further comprising performing the first assay that comprises:
(i) obtaining a first biological sample from the subject; and
(ii) measuring a first amount of cell-free nucleic acid molecules from the pathogen in the first biological sample.

12. The method of claim 11, wherein the measuring the first amount comprises measuring a copy number of the cell-free nucleic acid molecules from the pathogen in the first biological sample, or a first percentage of the cell-free nucleic acid molecules from the pathogen in the first biological sample.

13-15. (canceled)

16. The method of claim 11, wherein the first assay further comprises:
(iii) if the first amount is above a threshold, obtaining a second biological sample from the subject, and measuring a second amount of cell-free nucleic acid molecules from the pathogen in the second biological sample.

17. The method of claim 16, wherein the second biological sample is obtained about 4 weeks after the first biological sample.

18. The method of claim 16, wherein the interval between the first time point and the second time point is shorter if both the first amount and the second amount are above the threshold as compared to an interval if the second amount is below the threshold, and the interval between the first time point and the second time point is longer if the first amount is below the threshold as compared to an interval if the first amount is above the threshold.

19. (canceled)

20. The method of claim 16, wherein the interval between the first time point and the second time point is:
about 1 year if both the first amount and the second amount are above the threshold;
about 2 years if the second amount is below the threshold; or
about 4 years if the first amount is below the threshold.

21-37. (canceled)

38. The method of claim 1, wherein the first assay comprises determining the methylation status, the fragment size distribution, or the variant pattern of the cell-free nucleic acid molecules from the pathogen in the biological sample.

39. The method of claim 1, further comprising: calculating a risk score for the subject to develop the pathogen-associated disorder using a classifier applied to a data input comprising the characteristic of the cell-free nucleic acid molecules from the pathogen in the biological sample, wherein the classifier is configured to apply a function to the data input comprising the characteristic of the cell-free nucleic acid molecules from the pathogen in the biological sample to generate an output comprising the risk score that evaluates the risk for the subject to develop the disorder.

40. The method of claim 39, wherein the classifier is trained with a labeled dataset.

41. The method of claim 1, further comprising performing the second assay at the second time point.

42. (canceled)

43. The method of claim 41, wherein the second assay comprises an assay of cell-free nucleic acid molecules from the subject, an invasive biopsy of the subject, endoscopic examination of the subject, or magnetic resonance imaging examination of the subject.

44. A method of analyzing nucleic acid molecules from a biological sample of a subject, comprising:
a) obtaining, in a computer system, sequence reads of cell-free nucleic acid molecules from the biological sample of the subject, wherein the biological sample comprises cell-free nucleic acid molecules from the subject and potentially from a pathogen;
b) aligning, in the computer system, the sequence reads of the cell-free nucleic acid molecules to a reference genome of the pathogen; and
c) identifying, in the computer system, a variant pattern of the cell-free nucleic acid molecules from the pathogen, wherein the variant pattern characterizes a nucleotide variant of the sequence reads mapped to the reference genome of the pathogen at each of a plurality of variant sites on the reference genome of the pathogen, wherein the plurality of variant sites comprises at least 30 sites across the reference genome of the pathogen, and wherein the variant pattern indicates a status of, or a risk for, a pathogen-associated disorder in the subject.

45-76. (canceled)

77. A non-transitory computer-readable medium comprising machine executable code that, upon execution by one or more computer processors, implements the method of claim 1.

78. A computer product comprising a non-transitory computer readable medium storing a plurality of instructions for controlling a computer system to perform operations of the method of claim 1.

79. A system comprising:
the computer product of claim 79; and
one or more processors for executing instructions stored on the computer readable medium.
